(19) United States
(12) Patent Application Publication
(10) Pub. No.: US 2024/0366581 A1
(43) Pub. Date: Nov. 7, 2024

(54) BENZIMIDAZOLE PYRIDINE DERIVATIVES

(71) Applicant: Hoffmann-La Roche Inc., Little Falls, NJ (US)

(72) Inventors: Katrin GROEBKE ZBINDEN, Liestal (CH); Wolfgang HAAP, Lörrach (DE); Lukas KREIS, Baden (CH); Kin-Chun Thomas LUK, North Caldwell, NJ (US); Steven Gregory MISCHKE, Waltham, MA (US); Philippe PFLIEGER, Schwoben (FR)

(73) Assignee: Hoffmann-La Roche Inc., Little Falls, NJ (US)

(21) Appl. No.: 18/777,331

(22) Filed: Jul. 18, 2024

Related U.S. Application Data
(63) Continuation of application No. PCT/EP2023/051053, filed on Jan. 18, 2023.

(30) Foreign Application Priority Data
Jan. 19, 2022 (EP) 22152241.0

(51) Int. Cl. A61K 31/4439; A61K 31/506; C07D 401/04; C07D 401/14

(52) U.S. Cl. CPC A61K 31/4439; A61K 31/506; C07D 401/04; C07D 401/14

(57) ABSTRACT

The invention relates to a compound of formula (I)

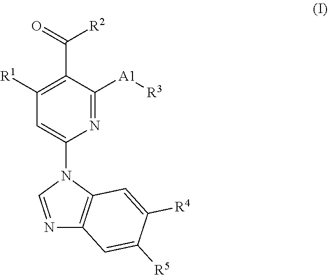

wherein A1, R¹, R², R³, R⁴ and R⁵ are as defined in the description and in the claims. The compound of formula (I) can be used as a medicament for treatment of indications associated with Salt-inducible kinases (SIK) such as of rheumatoid arthritis, juvenile rheumatoid arthritis, non-alcoholic steatohepatitis (NASH), primary sclerosing cholangitis, giant cell vasculitis, inflammatory bowel diseases (IBD), atherosclerosis, type 2 diabetes or glomerulonephritis.

### CROSS REFERENCE TO RELATED APPLICATIONS

This application is a continuation of International Application No. PCT/EP2023/051053, filed Jan. 18, 2023, which claims priority to European Patent Application No. 22152241.0 filed Jan. 19, 2022 both of which are incorporated herein by reference in its entirety.

### FIELD OF THE INVENTION

The invention relates to organic compounds useful for therapy and/or prophylaxis in a mammal, and in particular to compounds that modulate SIK activity.

### BACKGROUND OF THE INVENTION

Salt-inducible kinases (SIK) belong to a subfamily of AMP-activated protein kinases (AMPK) called AMPK-related kinases. There are three members, named SIK1, SIK2 and SIK3, that are broadly expressed. Their major biological role is to modify gene expression by controlling the phosphorylation and subcellular localization of two key classes of transcriptional regulatory factors: CRTCs (cAMP-regulated transcriptional coactivators) and class IIa HDACs (Histone deacetylases). Indeed, in basal state, both CRTCs and HDACs are phosphorylated by SIK kinases, and sequestered in the cytoplasm through interactions with their cytoplasmic chaperones 14-3-3. In response to extracellular cues that usually increase intracellular levels of cAMP, the SIK kinases' activity is inhibited, CRTCs and HDACs are no longer phosphorylated and are hence released from 14-3-3. They can therefore translocate into the nucleus and regulate gene expression (reviewed in Wein et al., Trends Endocrinol Metab. 2018 October; 29(10):723-735).

In macrophages, the inhibition of SIK kinases leads to 1) CRTC3 shuttling to the nucleus and increasing the transcription of IL-10; and 2) translocation of HDAC4/5 to the nucleus and subsequent deacetylation of NF-κB resulting in decreased transcription of pro-inflammatory cytokines (Clark et al., Proc Natl Acad Sci USA. 2012 Oct. 16; 109(42):16986-91.).

Macrophages are critical to maintaining tissue homeostasis, mediating inflammation, and promoting the resolution of inflammation. To achieve this diversity of function, macrophages have the ability to “polarize” differently in response to environment cues. The two extreme phenotypes along their activation state continuum are the “M1” or “pro-inflammatory macrophages” and the “M2” or “pro-resolution macrophages”.

Strikingly, the inhibition of intracellular SIK kinases overrides these extracellular macrophage polarization signals and pushes them toward a pro-resolution phenotype. This comes with an increase in IL-10 (by interfering with the SIK-CRTC3 pathway) and a concomitant decrease in TNF-α, IL-12 and IL-6 (by interfering with the SIK-HDAC4/5 and NF-κB pathway). The high levels of IL-10 and low levels of pro-inflammatory cytokines upon SIK inhibition will promote resolution of inflammation. The exploration of the SIK pathway has initially been described in macrophages (Clark et al., Proc Natl Acad Sci USA. 2012 Oct. 16; 109(42):16986-91) and dendritic cells (Sundberg et al., Proc Natl Acad Sci USA. 2014 Aug. 26; 111(34):12468-73) and the therapeutic potential of pan-SIK inhibitors has been confirmed in a mouse LPS (lipopolysaccharide) challenge model (Sundberg et al., ACS Chem Biol. 2016 Aug. 19; 11(8):2105-11) and in colitis models (Fu et al., Inflamm Bowel Dis. 2021 Oct. 20; 27(11):1821-1831). SIKs have since been shown to be important players in the functions of several immune cells, including mast cells (Darling et al., J Biol Chem. 2021 January-June; 296:100428). Importantly, SIK1 is poorly expressed in macrophages and one embodiment of the invention are SIK2/3 inhibitors sparing SIK1, thus limiting potential SIK1-related toxicities.

SIK inhibitors have a high therapeutic potential in diseases that are 1) characterized by pro-inflammatory macrophage influx in the tissues and impaired tissue homeostasis and healing, or 2) where anti-TNF therapies are beneficial (partially or fully) or with insufficient levels of the IL10. Diseases with an inflammatory macrophage signature are e.g. rheumatoid arthritis, juvenile rheumatoid arthritis, NASH, primary sclerosing cholangitis, giant cell vasculitis and inflammatory bowel diseases (“IBD”), atherosclerosis, type 2 diabetes and glomerulonephritis.

Diseases with a proven link to IL-10 and TNF-α are IBD. Genetic alterations that reduce the function of IL-10 (such as SNPs in IL-10 or its receptor) are associated with an increased risk for IBD in humans. In addition, anti-TNF therapies are successful but only a subset of IBD patients are responsive and much of this limited responsiveness is lost over time. The described dual effect of SIK inhibitors (increased IL-10 and decreased TNF-α) make them particularly pertinent for the treatment of IBD.

All three SIK kinase isoforms are expressed broadly in human tissues with the highest expression observed in skin and adipose tissues for SIK1, adipose tissue for SIK2 and testis and brain for SIK3. Similarly to their role in macrophages, SIKs in these cells phosphorylate CRTCs and class II HDCAs in response to extracellular signals, which subsequently change the expression of several cellular factors.

In addition to their physiological roles, reports have linked dysregulation of SIK expression to a few diseases. For example, SIK2 has been described as a risk locus for primary sclerosing cholangitis, a fibrotic disease regularly associated with IBD. In addition, SIK2 and SIK3 expression is higher in ovarian and prostate cancers and correlated with poor survival (Miranda et al., Cancer Cell. 2016 Aug. 8; 30(2):273-289; Bon et al., Mol Cancer Res. 2015 April; 13(4):620-635).

As of today many diseases caused by dysregulation of the innate immune system lack efficient therapies and there is a high unmet medical need for new therapies. The present invention relates to a novel compounds that are highly active SIK inhibitors for the treatment of inflammatory, allergic and autoimmune diseases. In addition to inflammation, allergic and autoimmune diseases, SIK inhibitors can thus also be of potential relevance in cancer, metabolic diseases, bone density dysregulation diseases, pigmentation-related diseases or cosmetology, fibrotic diseases and depressive disorders.

### SUMMARY OF THE INVENTION

The invention relates in particular to a compound of formula (I)

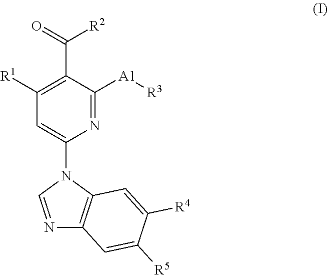

wherein
R¹ is hydrogen or alkoxy;
R² is hydrogen, alkyl, amino, alkylamino, dialkylamino, haloalkyl, haloalkylamino cycloalkylamino, hydroxy, alkoxy, cycloalkyl, cycloalkyloxy or haloalkoxy;
A1 is —O—, —NR⁶— or a bond;
R⁶ is hydrogen or alkyl;
R³ is alkyl, haloalkyl, hydroxyalkyl, heterocycloalkyl, heteroaryl, phenyl, heteroarylalkyl, phenylalkyl, cycloalkyl, cycloalkylalkyl, (amino)(phenyl)alkyl, (amino)(halophenyl)alkyl or (amino)(heteroaryl)alkyl, wherein heterocycloalkyl, heteroaryl, phenyl, heteroarylalkyl, phenylalkyl, cycloalkyl and cycloalkylalkyl are optionally substituted with 1, 2 or 3 substituents independently selected from R⁷;
each R⁷ is independently selected from alkoxy, alkylamino, alkyl, aminocarbonyl, amino, cyano, cycloalkylamino, haloalkyl, halocycloalkyl, halogen, heteroaryl, hydroxycarbonylamino, alkoxyalkyl, alkylaminocarbonyl, alkylsulfonyl, aminocarbonyl, hydroxy, cycloalkylalkyl, haloalkoxy, heterocycloalkyl and cycloalkyl;
R⁴ is hydrogen, alkyl, halogen, cyano, haloalkyl, alkoxy, alkoxyalkyl, dialkylaminoalkyl, dialkylamino, alkylamino, alkylaminoalkyl, cycloalkyl, cycloalkylamino, cycloalkyloxy, cycloalkylalkyl, heteroarylalkyl, heteroarylamino, heteroaryloxy, heterocycloalkyl, heterocycloalkylamino, heterocycloalkyloxy or heterocycloalkylalkyl; wherein cycloalkyl, cycloalkylamino, cycloalkyloxy, cycloalkylalkyl, heteroarylalkyl, heteroarylamino, heteroaryloxy, heterocycloalkyl, heterocycloalkylamino, heterocycloalkyloxy and heterocycloalkylalkyl are optionally substituted with 1, 2 or 3 substituents independently selected from R⁸;
each R⁸ is independently selected from alkyl, halogen, cyano, alkylsulfonyl, alkylaminocarbonyl, heterocycloalkyl and alkoxyheterocycloalkylalkyl;
R⁵ is hydrogen, alkyl, halogen, cyano, haloalkyl, alkoxy, alkoxyalkyl, dialkylaminoalkyl, dialkylamino, alkylamino, alkylaminoalkyl, alkylsulfonyl, cycloalkyl, cycloalkylamino, cycloalkyloxy, cycloalkylalkyl, heteroarylalkyl, heteroarylamino, heteroaryloxy, heterocycloalkyl, heterocycloalkylamino, heterocycloalkyloxy or heterocycloalkylalkyl; wherein cycloalkyl, cycloalkylamino, cycloalkyloxy, cycloalkylalkyl, heteroarylalkyl, heteroarylamino, heteroaryloxy, heterocycloalkyl, heterocycloalkylamino, heterocycloalkyloxy and heterocycloalkylalkyl are optionally substituted with 1, 2 or 3 substituents independently selected from R⁹;
or R⁴ and R⁵ together with the carbon they are attached to form a 5 to 7 membered heterocyclic ring optionally substituted with one, two or three substituents independently selected from alkyl, cyano, halogen, haloalkyl, alkoxy, heteroaryl and alkylheteroaryl;
each R⁹ is independently selected from alkoxy, halogen, dialkylaminocarbonyl, alkyl, alkoxyalkoxy, alkoxyheterocycloalkylalkyl, alkoxyheterocycloalkylcarbonyl, haloalkyl, haloalkoxy, heterocycloalkylalkoxy, heterocycloalkyl, heterocycloalkyloxy, hydroxy, alkylheterocycloalkyl, alkylheterocycloalkylalkyl, heterocycloalkylalkyl, alkylsulfonyl, (alkyl)heterocycloalkyl, alkylheterocycloalkyloxy, heterocycloalkylheterocycloalkyl, (heterocycloalkyl)heterocycloalkyl, CH₃—O—(CH₂—CH₂—O)₇—, alkylaminocarbonyl and cyano;
or a pharmaceutically acceptable salt thereof.

### DETAILED DESCRIPTION OF THE INVENTION

In the present description the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C1-C8 alkyl groups include, but are not limited to, methyl, ethyl, propyl, isopropyl, butyl, isobutyl, tert.-butyl, the isomeric pentyls, the isomeric hexyls, the isomeric heptyls and the isomeric octyls, particularly methyl, ethyl, propyl, butyl and pentyl. Particular examples of alkyl are methyl, ethyl, propyl, isopropyl, butyl and isobutyl. Methyl, ethyl, propyl and butyl, like isobutyl, are further particular examples of “alkyl” in the compound of formula (I).

The term “cycloalkyl”, alone or in combination, signifies a cycloalkyl ring with 3 to 8 carbon atoms and particularly a cycloalkyl ring with 3 to 6 carbon atoms. Examples of cycloalkyl include, but are not limited to, cyclopropyl, cyclobutyl, cyclopentyl, cyclohexyl, cycloheptyl and cyclooctyl. Particular examples of “cycloalkyl” are cyclopropyl and cyclobutyl.

The term “heterocycloalkyl”, alone or in combination, denotes a monovalent saturated or partly unsaturated mono-, bi- or tricyclic ring system of 4 to 12 ring atoms, comprising 1, 2, or 3 ring heteroatoms selected from N, O and S, the remaining ring atoms being carbon. Bicyclic means consisting of two cycles having one or two ring atoms in common. “Hetercycloylkyl” may comprise a carbonyl group, wherein the carbon of the carbonyl group is part of the ring system. The ring system can be attached to the remaining compound via an atom selected from C, N, S and O, in particular via a N atom (“N-heterocycloalkyl). Examples of “heterocycloalkyl” include, but are not limited to, morpholino, morpholin-4-yl, pyrrolidinyl, pyrrolidin-1-yl, pyrrolidin-3-yl, piperidinyl, 1-piperidyl, 4-piperidyl, 2-oxopyrrolidin-1-yl, piperazinyl, piperazin-1-yl, azetidinyl, azetidin-1-yl, [(1S,5R,7R)-4-oxo-3-oxa-9-azatricyclo[5.3.0.01,5]decan-9-yl], [3-oxo-piperazin-1-yl], (1,1-dioxo-1,2-thiazolidin-2-yl), (4,5,6,7-tetrahydropyrazolo[4,3-c]pyridin-1-yl), (3-oxo-1,5,6,8-tetrahydrooxazolo[3,4-a]pyrazin-7-yl), [rac-(3aR,6aS)-2,3,3a,5,6,6a-hexahydro-1H-pyrrolo[3,2-b]pyrrol-4-yl], [rac-(3aS,6aR)-2,3,3a,5,6,6a-hexahydro-1H-pyrrolo[3,2-b]pyrrol-4-yl], (4-oxo-6,7-dihydro-5H-pyrazolo[1,5-a]pyrazin-3-yl), (6,7-dihydro-4H-pyrazolo[4,3-c]pyridin-1-yl), (4,7-diazaspiro[2.5]octan-7-yl), (2-oxa-5,8-diazaspiro[3.5]nonan-8-yl), 3-azabicyclo[3.2.0]heptan-3-yl), (5-azaspiro[2.4]heptan-5-yl), (2-azabicyclo[2.2.1]heptan-2-yl), 4-oxa-7-azaspiro[2.5]octan-7-yl, (3-azabicyclo[3.1.0]hexan-3-yl), (6,7-dihydro-4H-pyrazolo[4,3-c]pyridin-1-yl), 2-oxa-7-azaspiro[3.4]octan-7-yl, (2-oxo-1-piperidyl), (2,3-dihydropyridazino[4,5-b][1,4]oxazin-8-yl), pyrrolidin-1-yl, 2-oxo-pyrimidin-4-yl, morpholinoethyl, 2-oxa-5-azaspiro[3.4]octan-5-yl, oxetan-3-yl, (2-oxo-1-piperidyl), 2-oxo-4-piperidyl, 5-oxo-pyrrolidin-3-yl, 2-oxa-5-azaspiro[3.4]octan-5-yl, (7,8-dihydro-5H-pyrano[4,3-c]pyridazin-3-yl), [rac-(4aS,7aR)-4-methyl-2,3,4a,5,7,7a-hexahydropyrrolo[3,4-b][1,4]oxazin-6-yl] and [rac-(3aS,6aS)-6-oxo-2,3,3a,4,5,6a-hexahydropyrrolo[2,3-c]pyrrol-1-yl]. Particular examples of “heterocycloalkyl” is pyrrolidin-1-yl and pyrrolidin-3-yl. In one particular embodiment, heterocycloalkyl is “N-heterocycloalkyl”.

The term “heteroaryl”, alone or in combination, signifies an aromatic mono- or bicyclic ring system with 5 to 12 ring atoms, comprising 1, 2, 3 or 4 heteroatoms each independently selected from N, O and S, the remaining ring atoms being carbon. The ring system can be attached to the remaining compound via an atom selected from C, N, S and O, in particular via a N atom (“N-heteroaryl). Examples of heteroaryl include, but are not limited to, pyrazolyl, pyrazol-1-yl, pyrazol-3-yl, pyrazol-4-yl, pyridinyl, 2-pyridyl, 3-pyridyl, 4-pyridyl, pyridazinyl, pyridazin-3-yl, pyridazin-4-yl, pyrazinyl, pyrazin-2-yl, isoxazolyl, isoxazol-3-yl, isoxazol-4-yl, pyrimidinyl, pyrimidin-5-yl, benzotriazolyl, 1H-benzotriazol-4-yl, furanyl, furyl, 2-furyl, 3-furyl, [6-oxo-1H-pyridazin-5-yl], triazolyl, triazol-1-yl, triazol-2-yl, 2-oxo-4-pyridyl, pyrimidin-2-yl, pyrimidin-5-yl, (1,3,4-oxadiazol-2-yl), (1,3,4-thiadiazol-2-yl), (1,2,4-triazin-3-yl), 2-oxo-pyrimidin-4-yl, (1-methyl-2-oxo-3-pyridyl) and (2,3-dihydropyridazino[4,5-b][1,4]oxazin-8-yl). Particular examples of “heteroaryl” are pyrazol-1-yl, pyrazol-4-yl, pyridazin-3-yl ane pyrimidin-5-yl. In one particular embodiment, heteroaryl is “N-heteroaryl”.

The term “alkoxy” or “alkyloxy”, alone or in combination, signifies a group of the formula alkyl-O— in which the term “alkyl” has the previously given significance, such as methoxy, ethoxy, n-propoxy, isopropoxy, n-butoxy, isobutoxy, sec-butoxy and tert.-butoxy. Particular examples of “alkoxy” are methoxy and ethoxy.

The term “oxy”, alone or in combination, signifies the —O— group.

The term “oxo”, alone or in combination, signifies the ═O group.

The terms “halogen” or “halo”, alone or in combination, signifies fluorine, chlorine, bromine or iodine and particularly fluorine, chlorine or bromine, more particularly fluorine. The term “halo”, in combination with another group, denotes the substitution of said group with at least one halogen, particularly substituted with one to five halogens, particularly one to four halogens, i.e. one, two, three or four halogens.

The term “haloalkyl”, alone or in combination, denotes an alkyl group substituted with at least one halogen, particularly substituted with one to five halogens, particularly one to three halogens, more particularly two to three halogens. Particular “haloalkyl” are fluoromethyl, fluoroethyl, fluoropropyl, fluorobutyl, difluoromethyl, difluoroethyl, trifluoromethyl and trifluoroethyl.

The term “haloalkoxy”, alone or in combination, denotes an alkoxy group substituted with at least one halogen, particularly substituted with one to five halogens, particularly one to three halogens. Particular “haloalkoxy” are fluoromethoxy, fluoroethoxy and fluoropropyloxy.

The terms “hydroxyl” and “hydroxy”, alone or in combination, signify the —OH group.

The term “carbonyl”, alone or in combination, signifies the —C(O)— group.

The term “amino”, alone or in combination, signifies the primary amino group (—NH₂), the secondary amino group (—NH—), or the tertiary amino group (—N—).

The term “alkylamino” is alkyl group linked to a —NH— group. The term “dialkylamino” denotes two alkyl groups linked to a —N— atom.

The term “sulfonyl”, alone or in combination, signifies the —SO₂— group.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine. In addition these salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula (I) can also be present in the form of zwitterions. Particular pharmaceutically acceptable salts of compounds of formula (I) are the salts of trifluoroacetic acid, hydrochloric acid, formic acid, hydrobromic acid, sulfuric acid, phosphoric acid and methanesulfonic acid.

In the instance where R⁵ is optionally substituted (pyridazin-3-yl)amino, the compound of formula (Ia) can exist as a tautomer (Ia′), i.e. a structural isomer which interconverts with the compound of formula (I), in particular in solution.

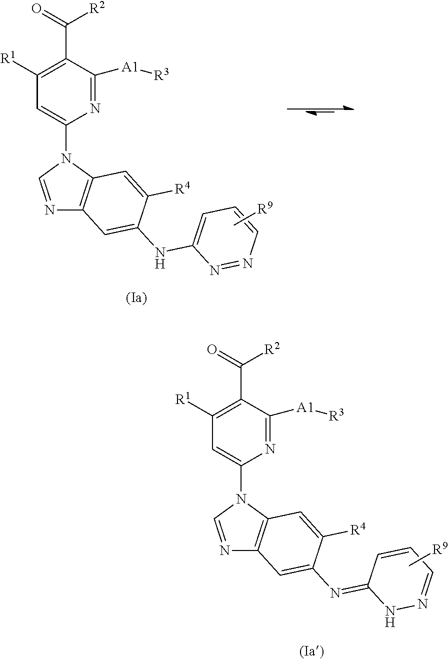

Other tautomeric forms of the compound of formula (I) can also exist and the corresponding tautomers form are to be considered as being encompasses by the compound of formula (I).

The compound of formula (I) wherein R² is hydrogen, alkyl, cycloalkyl or haloalkyl has a hydrate form (I″), which can be represented as follows:

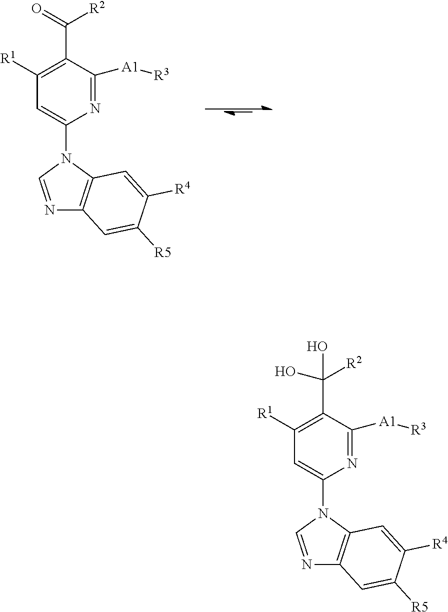

If one of the starting materials or compounds of formula (I) contain one or more functional groups which are not stable or are reactive under the reaction conditions of one or more reaction steps, appropriate protecting groups (as described e.g. in “Protective Groups in Organic Chemistry” by T. W. Greene and P. G. M. Wuts, 3^{rd} Ed., 1999, Wiley, New York) can be introduced before the critical step applying methods well known in the art. Such protecting groups can be removed at a later stage of the synthesis using standard methods described in the literature. Examples of protecting groups are tert-butoxycarbonyl (Boc), 9-fluorenylmethyl carbamate (Fmoc), 2-trimethylsilylethyl carbamate (Teoc), carbobenzyloxy (Cbz) and p-methoxybenzyloxycarbonyl (Moz).

The compound of formula (I) can contain several asymmetric centers and can be present in the form of optically pure enantiomers, mixtures of enantiomers such as, for example, racemates, mixtures of diastereoisomers, diastereoisomeric racemates or mixtures of diastereoisomeric racemates.

The term “asymmetric carbon atom” means a carbon atom with four different substituents. According to the Cahn-Ingold-Prelog Convention an asymmetric carbon atom can be of the “R” or “S” configuration.

Furthermore, the invention includes all optical isomers, i.e. diastereoisomers, diastereomeric mixtures, racemic mixtures, all their corresponding enantiomers and/or tautomers as well as their solvates, wherever applicable, of the compound of formula (I).

If desired, racemic mixtures of the compound of the invention may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography.

In the embodiments, where an optically pure enantiomer is provided, optically pure enantiomer means that the compound contains>90% of the desired isomer by weight, particularly >95% of the desired isomer by weight, or more particularly >99% of the desired isomer by weight, said weight percent based upon the total weight of the isomer of the compound. A chirally pure or chirally enriched compound may be prepared by chirally selective synthesis or by separation of enantiomers. The separation of enantiomers may be carried out on the final product or alternatively on a suitable intermediate.

Furthermore, the invention includes all substituents in their corresponding deuterated form, wherever applicable, of the compound of formula (I).

Furthermore, the invention includes all substituents in their corresponding tritiated form, wherever applicable, of the compound of formula (I).

A certain embodiment of the invention relates to the compound of formula (I) as described herein, or a pharmaceutically acceptable salt thereof, wherein at least one substituent comprises at least one radioisotope. Particular examples of radioisotopes are ²H, ³H, ¹³C, ¹⁴C and ¹⁸F.

### General Synthetic Schemes

The synthesis of the compound of formula (I) can, for example, be accomplished according to schemes 1 to 6 and according to methods known to those skilled in the art.

### Scheme 1

In scheme 1, the synthesis of a compound of formula (I-a) is described. R^{a} is alkyl; R^{a′} is hydrogen. The compound of formula (I-a) is a compound of formula (I), wherein A1 a bond; R¹ is hydrogen; R² is amino or aminoalkyl; R³ is phenyl optionally substituted with one, two or three substituents independently selected from R⁷; R⁴ is alkoxy; R⁵ is alkoxy; each R⁷ is indecently selected from alkoxy and halogen.

In scheme 1, the synthesis of a compound of formula (I-b) is described. R^{b} is phenyl optionally substituted with R⁷. The compound of formula (I-b) is a compound of formula (I), wherein A1 a —O—; R¹ is hydrogen; R² is amino; R³ is phenyl optionally substituted with one, two or three substituents independently selected from R⁷; R⁴ is alkoxy; R⁵ is alkoxy; each R⁷ is indecently selected from alkoxy and halogen.

In scheme 1, the synthesis of a compound of formula (I-c) is described. R^{c} is phenyl optionally substituted with R⁷. The compound of formula (I-c) is a compound of formula (I), wherein A1-NH—; R¹ is hydrogen; R² is amino; R³ is phenyl optionally substituted with one, two or three substituents independently selected from R⁷; R⁴ is alkoxy; R⁵ is alkoxy; each R⁷ is indecently selected from alkoxy and halogen.

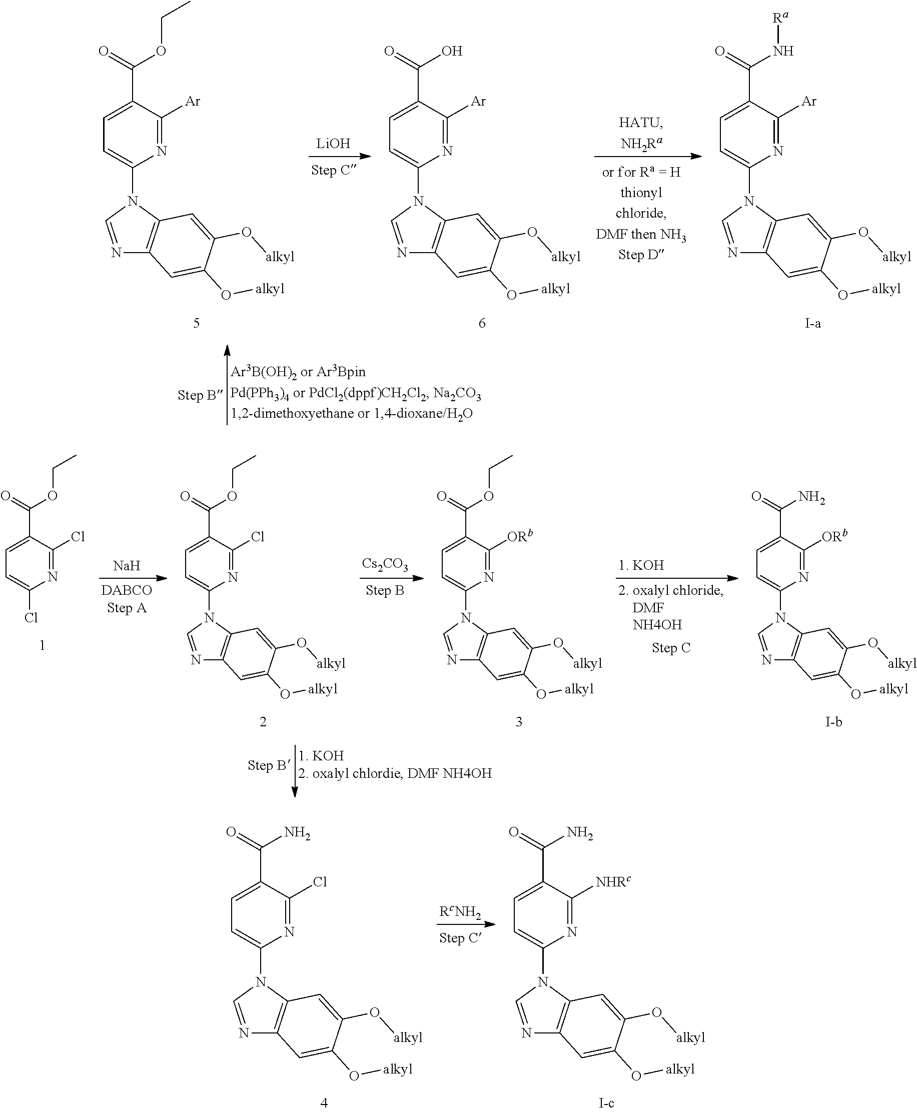

Step A: Ethyl 2,6-dichloronicotinate 1 can be submitted to an aromatic nucleophilic substitution with 5,6-dialkoxy-1H-benzo[d]imidazole using a suitable base such as for instance NaH or DABCO, and a suitable solvent such as for instance DMF at around 0° C. to yield intermediate 2.

Step B: Intermediate 2 can further be converted to 3 with a substituted phenol in the presence of a suitable base such as for instance Cs₂CO₃ in a suitable solvent such as for instance DMF at around 50° C.

Step C: A primary amide can then be introduced through saponification with a suitable base such as for instance KOH in a suitable solvent (THF, CH₃CN, MeOH, H₂O, or a mixture thereof) and subsequent amide coupling with oxalyl chloride and DMF in a suitable solvent such as for instance DCM and a suitable amine source such as for instance NH₄OH, to yield the compound I-b.

Step B′: Similarly primary amide 4 can be obtained through saponification with a suitable base such as for instance KOH in a mixture of solvents (THF, CH₃CN, MeOH, H₂O) and subsequent amide coupling with oxalyl chloride and DMF in a suitable solvent such as for instance DCM and a suitable amine source such as for instance NH₄OH.

Step C′: Subsequent coupling of 4 with aniline in ethylene glycol at 160° C. affords the compound I-c.

Step B″: A palladium-catalyzed cross-coupling reaction (Suzuki-Miyaura) of 2 with the corresponding aryl boronic acid or aryl pinacol borane catalytic Pd(PPh₃)₄ pr PdCl₂(dppf)CH₂Cl₂ and a suitable base (e.g. Na₂CO₃) in a suitable solvent (e.g. DME, 1,4-dioxane and H₂O) while heating (e.g. MW at 120° C. or 90° C.) yields intermediate 5.

Step C″: Saponification of 5 with a suitable base such as for instance LiOH in a suitable solvent such as for instance THF/MeOH yields the free carboxylic acid 6.

Step D″: 6 can be coupled with a primary amide in the presence of HATU to afford a secondary amide I-a, while the reaction with thionyl chloride and DMF, followed by conversion with ammonia yields the corresponding primary amides I-a′.

### Scheme 2

In scheme 2, the synthesis of a compound of formula (J-d) is described. The compound of formula (J-d) is a compound of formula (I), wherein A1-NH—; R¹ is hydrogen; R² is amino; R³ is arylalkyl or aryl optionally substituted with one, two or three substituents independently selected from R⁷; R⁴ is alkoxy; R⁵ is alkoxy; each R⁷ is independently selected from alkoxy and halogen.

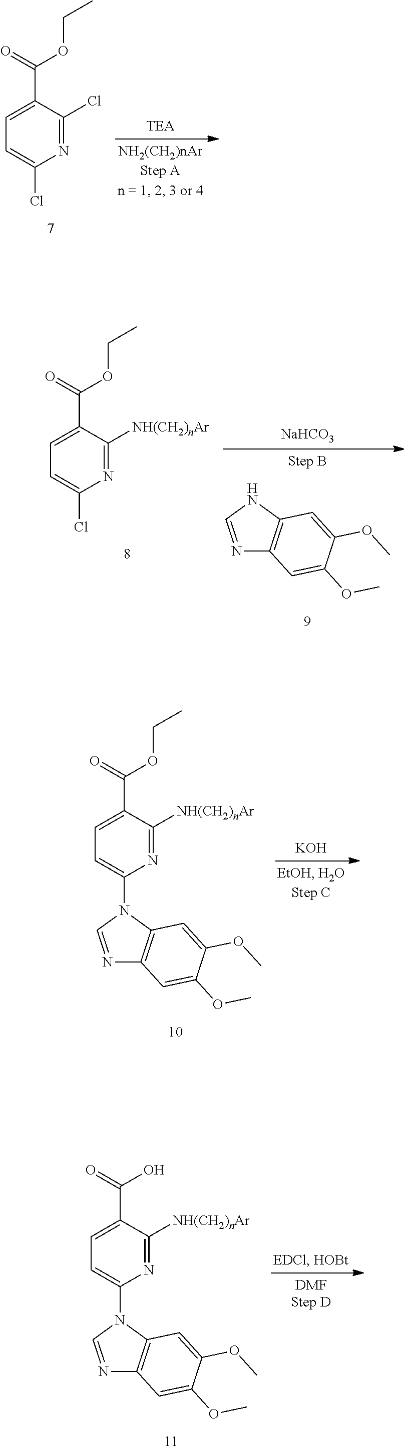

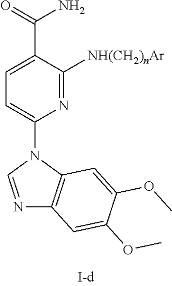

Step A: 2,6-dichloronicotinate 7 can be reacted with an alkyl- or benzylamine (?) in the presence of a suitable base such as for instance TEA in a suitable solvent such as for instance 2-methoxyethanol at around 80° C. to yield intermediate 8.

Step B: The intermediate 8 can be submitted to an aromatic nucleophilic substitution with 5,6-dimethoxy-1H-benzo[d]imidazole 9 using a suitable base such as for instance NaHCO₃ in a suitable solvent such as for instance DMSO at around 130° C. to yield 10.

Step C: Saponification of the ester group of 10 with a suitable base such as for instance KOH in a suitable solvent such as for instance an EtOH/H₂O mixture affords acid 11.

Step D: The acid 9 can be converted to the corresponding amide of formula (I-d) using for instance EDCI and HOBt in a suitable solvent such as e.g. DMF at around 50° C.

### Scheme 3

In scheme 3, the synthesis of a compound of formula (I-e) is described. The compound of formula (I-e) is a compound of formula (I), wherein A1 is a bond; R¹ is hydrogen; R² is alkoxy; R³ is N-heterocycloalkyl; R⁴ is alkoxy; R⁵ is alkoxy.

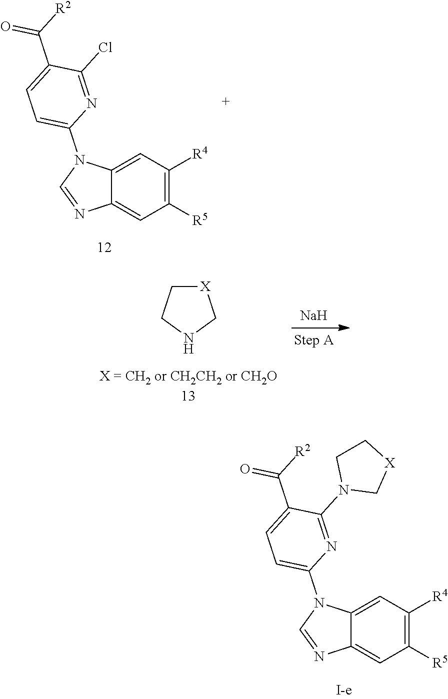

Step A: Chlorpyridine derivative 12 can be substituted with a saturated N-heterocycle 13 in the presence of a strong base (such as for instance NaH, or Cs₂CO₃ or other carbonates) in a polar solvent (such as for instance DMF, DMA, NMP or DMSO) to yield a compound of formula (I-e).

### Scheme 4

In scheme 4, the synthesis of a compound of formula (I-f) is described. The compound of formula (I-f) is a compound of formula (I), wherein A1 is a bond; R¹ is hydrogen; R² is alkoxy; R³ is N-heterocycloalkyl; R⁴ is alkoxy; R⁵ is alkoxy.

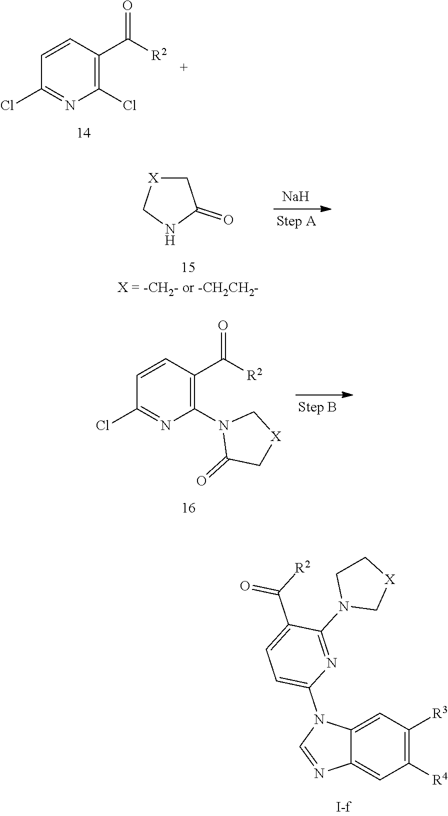

Step A: Alkyl 2,6-dichloronicotinate 14 can be reacted with a cyclic amide 15 in the presence of a suitable base such as for instance NaH in a suitable solvent such as for instance DMF at around 0° C. to yield intermediate 16.

Step B: Further substitution of intermediate 16 with a 5,6-disubstituted benzimidazole in the presence of a strong base such NaH in a polar solvent (e.g. DMF or DMSO) at around 0° C. yields intermediate the compound of formula (I-f).

### Scheme 5

In scheme 5, the synthesis of a compound of formula (J-g) and a regioisomer thereof (J-g′) is described. The compound of formula (J-g) is a compound of formula (I), wherein A1 is a bond; R¹ is hydrogen; R² is alkyl or alkoxy; R³ is pyrazol-1-yl optionally substituted with one, two or three substituents independently selected from R⁷; R⁴ is hydrogen; R⁵ is (pyridazin-3-yl)amino optionally substituted with R⁹; each R⁷ is independently selected from alkyl, cyano, haloalkyl, alkoxy, alkylaminocarbonyl and alkylsulfonyl; R⁹ is alkyl. The compound of formula (J-g′) is a compound of formula (I), wherein A1 is a bond; R¹ is hydrogen; R² is alkyl or alkoxy; R³ is pyrazol-1-yl optionally substituted with one, two or three substituents independently selected from R⁷; R⁴ is (pyridazin-3-yl)amino optionally substituted with R⁸; R⁵ is hydrogen; each R⁷ is independently selected from alkyl, cyano, haloalkyl, alkoxy, alkylaminocarbonyl and alkylsulfonyl; R⁸ is alkyl.

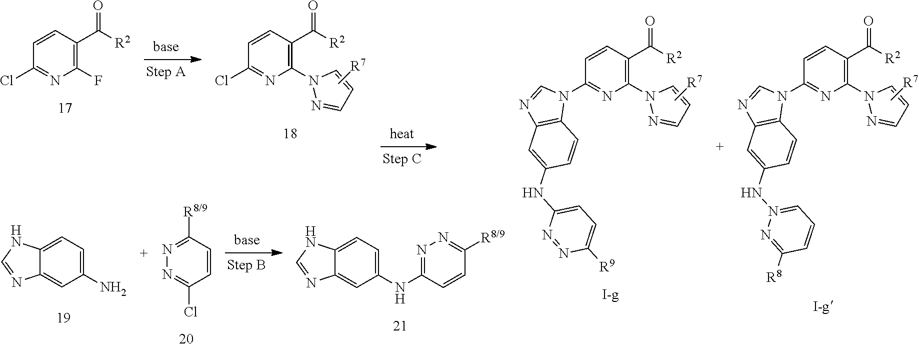

Step A: 1-(6-chloro-2-fluoro-3-pyridyl)alkanone (or a suitable derivate thereof) 17 can be reacted with a substituted pyrazole in the presence of a suitable organic or mineral base (e.g. DIPEA, DBU, K₂CO₃, Cs₂CO₃, or NaH) in a polar solvent (e.g. DMF, DMSO or THF) to yield intermediate 18.

Step B: Intermediate 21 can be obtained from the reaction of 5-aminobenzimidazole 19 and 3-chloro-alkylpyridazinyl 20 in a suitable solvent such as for instance iPrOH while heating to reflux.

Step C: The intermediates 18 and 21 can be combined in the presence of a suitable organic or mineral base (DIPEA, DBU, K₂CO₃, Cs₂CO₃, or NaH) in a suitable polar solvent (e.g. DMF, DMSO or THF) yielding the regioisomeric compounds of formula (I-g) and (I-g′) which can be separated by flash column chromatography.

### Scheme 6

In scheme 6, the synthesis of a compound of formula (I-h) and a regioisomer thereof (I-h′) is described. The compound of formula (I-h) is a compound of formula (I), wherein A1 is a bond; R¹ is hydrogen; R² is alkyl; R³ is pyrazol-1-yl optionally substituted with one, two or three substituents independently selected from R7; R⁴ is hydrogen; R⁵ is heteroarylamino, optionally substituted with R⁹; each R⁷ is independently selected from alkyl, cyano, haloalkyl, alkoxy, alkylaminocarbonyl and alkylsulfonyl; R⁹ is alkyl. The compound of formula (I-h′) is a compound of formula (I), wherein A1 is a bond; R¹ is hydrogen; R² is alkyl or alkoxy; R³ is pyrazol-1-yl optionally substituted with one, two or three substituents independently selected from R⁷; R⁴ is heteroarylamino optionally substituted with R⁸; R⁵ is hydrogen; each R⁷ is independently selected from alkyl, cyano, haloalkyl, alkoxy, alkylaminocarbonyl and alkylsulfonyl; R⁸ is alkyl.

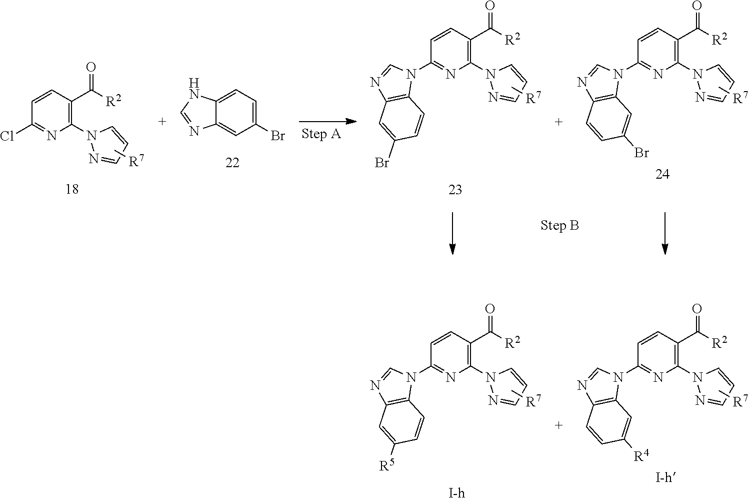

Step A: The regioisomeric intermediates 23 and 24 can be obtained similarly to the description in scheme 10, using intermediate 18 (from scheme 5) and 5-bromobenzimidazole 22 as the second reagent.

Step B: Introduction of a heteroarylamino group to yield a compound of formula (I-h) and the regioisomer (I-h′) can be performed via a Buchwald-Hartwig coupling. The reaction can be done using a suitable base such as for instance Cs₂CO₃ and t-Buxphos-Pd-G3 as palladium catalyst at around 90° C. or Cs₂CO₃ as base and [tBuBrettPhos Pd(allyl)]OTf as catalyst at around 80° C. The corresponding regioisomers I-h and I-h′ are separated by either flash chromatography or preparative high pressure liquid chromatography

The invention thus also relates to a process for the preparation of a compound according to the invention, comprising one of the following steps:
(a) the reaction of a compound of formula (B1) or (B2)

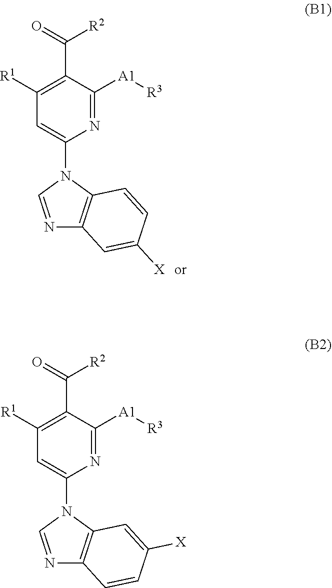

with an amine, in presence of a palladium catalyst and a base;
(b) the reaction of a compound of formula (C1)

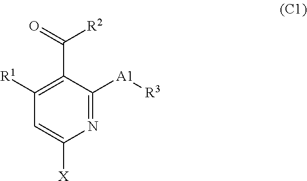

with a compound of formula (C2)

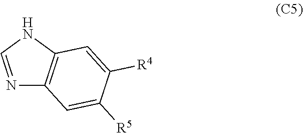

in presence of a base;
s (c) the reaction of a compound of formula (D1)

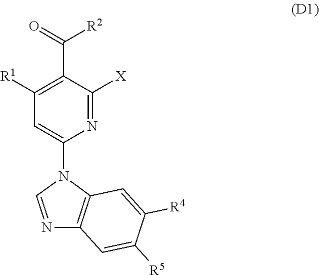

with an amine, in presence of a base; or
(d) the reaction of the compound of formula (D1)
with a compound (D2) in presence of a base and a palladium catalyst; wherein D2 is selected from (i) optionally substituted aryl boronic acid or ester, and (ii) optionally substituted heteroaryl boronic acid or ester;
wherein A1, A2, A3, R¹, R², R³, R⁴ and R⁵ are as defined above, R^{a} is alkyl or cycloalkyl, R^{b} is hydrogen or alkyl, R^{c} is alkyl or cycloalkyl, and X is halogen.

The amine of step (a) can be arylamine, heteroarylamine, alkylamine, cycloalkylamine or heterocycloalkylamine.

Conveniently, the palladium catalyst of step (a) can be selected from QPhosPd(crotyl)Cl, t-BuXphos-Pd-G3, RuPhos-Pd-G3, [tBuBrettPhos Pd(allyl)]OTf and Pd₂(dba)₃. Advantageously, the palladium catalyst is t-BuXphos-Pd-G3.

Conveniently, the base of step (a) can be selected from K₃PO₄, Na₂CO₃, K₂CO₃, Cs₂CO₃ and KOAc. Advantageously, the base is Cs₂CO₃.

Conveniently, the solvent of step (a) can be selected from DMF, DME, DMA, toluene, 1,4-dioxane and H₂O, or a mixture thereof. Advantageously, the solvent is 1,4-dioxane.

Convenient conditions for step (a) are between around 20° C. to around 280° C., in particular between around 40° C. to around 230° C., more particular between around 60° C. to around 180° C. during 1-24 hrs, advantageously during 1-12 hrs.

In step (a), X is conveniently chloro or bromo, particularly bromo

Conveniently, the base of step (b) can be selected from DBU, DIPEA, TEA, K₃PO₄, Na₂CO₃, NaHCO₃, K₂CO₃, Cs₂CO₃ and KOAc. Advantageously, the base is NaHCO₃ or K₂CO₃.

Conveniently, the solvent of step (b) can be selected from DMF, DMSO, IPA, THF, DME, DMA, toluene, 1,4-dioxane and H₂O, or a mixture thereof. Advantageously, the solvent is DMSO.

Conveniently, in step (b) a palladium catalyst can be used together with a suitable base selected from K₃PO₄, Na₂CO₃, K₂CO₃, Cs₂CO₃ and KOAc. Advantageously, the palladium catalyst can be selected from QPhosPd(crotyl)Cl, t-BuXphos-Pd-G3, RuPhos-Pd-G3, [tBuBrettPhos Pd(allyl)]OTf and Pd₂(dba)₃.

Convenient conditions for step (b) are between around −40° C. to around 220° C., in particular between around −30° C. to around 200° C., more particular between around −20° C. to around 180° C. during 1-24 hrs, advantageously during 1-12 hrs.

In step (b), X is conveniently chloro or bromo, particularly bromo.

The amine of step (c) can be optionally substituted heteroaryl selected from pyrrole, pyrazole and triazole.

Conveniently, the base of step (c) can be selected from DBU, DIPEA, TEA, K₃PO₄, Na₂CO₃, K₂CO₃, Cs₂CO₃ and KOAc.

Conveniently, the solvent of step (c) can be selected from DMF, DMSO, IPA, THF or a mixture thereof.

Convenient conditions for step (c) are between around −40° C. to around 200° C., in particular between around −20° C. to around 160° C., more particular between around 0° C. to around 120° C. during 1-24 hrs, advantageously during 1-12 hrs.

In step (c), X is conveniently bromo or chloro, particularly bromo.

Conveniently, the base of step (d) can be selected from K₃PO₄, Na₂CO₃, K₂CO₃, Cs₂CO₃ and KOAc.

Conveniently, the palladium catalyst of step (d) can be selected from Pd(PPh₃)₄, Pd₂(dba)₃, PdCl₂(dppf)·CH₂Cl₂ and Pd(OAc)₂. Advantageously, the palladium catalyst is Pd(PPh₃)₄ or PdCl₂(dppf)·CH₂Cl₂

Conveniently, the solvent of step (d) can be selected from DMF, DME, DMA, toluene, 1,4-dioxane and H₂O, or a mixture thereof.

Convenient conditions for step (d) are between around 20° C. to around 220° C., in particular between around 40° C. to around 200° C., more particular between around 60° C. to around 180° C. during 1-24 hrs, advantageously during 1-12 hrs.

In step (d), X is conveniently chloro and bromo, particularly chloro.

In step (d) benzene and heteroaryl are preferentially substituted with one, two or three substituents independently selected from halogen, amino, cyano, haloalkyl, halophenyl and heteroaryl.

The invention also relates to a compound according to the invention when manufactured according to a process of the invention.

### Pharmaceutical Compositions

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compound of formula (I) is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal, epidural and intranasal, and if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

### Example A

Film coated tablets containing the following ingredients can be manufactured in a conventional manner:

| | |
|---|---|
| Ingredients | Per tablet |

| | | | | |
|---|---|---|---|---|
| Kernel: | | | | |
| Compound of formula (I) or a | 10.0 | mg | 200.0 | mg |
| pharmaceutically acceptable salt thereof | | | | |
| Microcrystalline cellulose | 23.5 | mg | 43.5 | mg |
| Lactose hydrous | 60.0 | mg | 70.0 | mg |
| Povidone K30 | 12.5 | mg | 15.0 | mg |
| Sodium starch glycolate | 12.5 | mg | 17.0 | mg |
| Magnesium stearate | 1.5 | mg | 4.5 | mg |
| (Kernel Weight) | 120.0 | mg | 350.0 | mg |
| Film Coat: | | | | |
| Hydroxypropyl methyl cellulose | 3.5 | mg | 7.0 | mg |
| Polyethylene glycol 6000 | 0.8 | mg | 1.6 | mg |
| Talc | 1.3 | mg | 2.6 | mg |
| Iron oxide (yellow) | 0.8 | mg | 1.6 | mg |
| Titan dioxide | 0.8 | mg | 1.6 | mg |

The active ingredient is sieved and mixed with microcrystalline cellulose and the mixture is granulated with a solution of polyvinylpyrrolidone in water. The granulate is then mixed with sodium starch glycolate and magnesium stearate and compressed to yield kernels (uncoated tablet cores) of 120 or 350 mg respectively. The kernels are lacquered with an aq. solution/suspension of the above mentioned film coat.

### Example B

Capsules containing the following ingredients can be manufactured in a conventional manner:

| Ingredients | Per capsule | |
|---|---|---|
| Compound of formula (I) or a | 25.0 | mg |
| pharmaceutically acceptable salt thereof | | |
| Lactose | 150.0 | mg |
| Maize starch | 20.0 | mg |
| Talc | 5.0 | mg |

The components are sieved and mixed and filled into capsules of size 2.

### Example C

Injection solutions can have the following composition:

| | |
|---|---|
| Compound of formula (I) or a | 3.0 mg |
| pharmaceutically acceptable salt thereof | |
| Polyethylene glycol 400 | 150.0 mg |
| Acetic acid | q.s. ad pH 5.0 |
| Water for injection solutions | ad 1.0 ml |

The active ingredient is dissolved in a mixture of Polyethylene glycol 400 and water for injection (part). The pH is adjusted to 5.0 by addition of acetic acid. The volume is adjusted to 1.0 ml by addition of the residual amount of water. The solution is filtered, filled into vials using an appropriate overage and sterilized.

### Experimental Procedures

### Abbreviations

[tBuBrettPhos Pd(allyl)]OTf allyl(2-di-tert-butylphosphino-3,6-dimethoxy-2′,4′,6′-triisopropyl-1,1′-biphenyl)palladium(II) triflate (CAS #1798782-15-6)
2-Me-THF 2-methyl tetrahydrofuran
aq. aqueous
Boc tert-butyloxycarbonyl
CDI carbonyldiimidazole
DABCO 1,8-diazabicyclo[5.4.0]undec-7-ene
DAST diethylaminosulfur trifluoride
dba dibenzylideneacetone
DCM dichloromethane
DIAD diisopropyl azodicarboxylate
DIBAL-H diisobutylaluminium hydride
DIPEA N,N-diisopropylethylamine
DMF N,N-dimethylformamide
DMSO dimethyl sulfoxide
dppf 1,1′-ferrocenediyl-bis(diphenylphosphine)
dtbbpy 4,4′-Bis(1,1-dimethylethyl)-2,2′-bipyridine
dtbpy 4,4′-Di-tert-butyl-2,2′-dipyridyl
EDCI 1-ethyl-3-(3-dimethylaminopropyl)carbodiimide
equiv. equivalents
ESI electrospray ionization
Et ethyl
Et₂O diethyl ether
EtOAc ethyl acetate
EtOH ethanol
FA formic acid
HATU (1-[bis(dimethylamino)methylene]-1H-1,2,3-triazolo[4,5-b]pyridinium 3-oxide hexafluorophosphate
HMDS bis(trimethylsilyl)amine
HOBt hydoxybenzotriazole
HPLC high pressure liquid chromatography
IPA isopropyl alcohol
Ir[dF(CF₃)ppy]2(dtbpy)(PF₆) [4,4′-bis(1,1-dimethylethyl)-2,2′-bipyridine-N1,N1′]bis[3,5-difluoro-2-[5-(trifluoromethyl)-2-pyridinyl-N]phenyl-C]Iridium(III)hexafluorophosphate
LCMS high-performance liquid chromatography
LDA lithium diisopropylamide
mCPBA meta-chloroperoxybenzoic acid
Me methyl
MeOH methanol
Ms methanesulfonyl
NPLC normal phase liquid chromatography
PE petroleum ether
ppy 2-phenylpyridine
psi pounds per square inch
PTSA para toluene sulfonic acid
Qphos 1,2,3,4,5-Pentaphenyl-1′-(di-tert-butylphosphino)ferrocene
RT room temperature
RuPhos-Pd-G3 (2-Dicyclohexylphosphino-2′,6′-diisopropoxy-1,1′-biphenyl)[2-(2′-amino-1,1′-biphenyl)]palladium(II) methanesulfonate (CAS #1445085-77-7) sat. saturated
SFC supercritical fluid chromatography
sol. solution
TBD Triazabicyclodecene
TBDMS tert-butyldimethylsilyl
t-BuXphos-Pd-G3 [(2-Di-tert-butylphosphino-2′,4′,6′-triisopropyl-1,1′-biphenyl)-2-(2′-amino-1,1′-biphenyl)]palladium(II)methanesulfonate (CAS #1447963-75-8) TEA triethylamine
Tf triflyl
TFA trifluoroacetic acid
TFAA trifluoroacetic acid anhydride
THF tetrahydrofuran
TLC thin layer chromatography

### Example 1

### 2-(2-chlorophenoxy)-6-(5,6-dimethoxybenzimidazol-1-yl)pyridine-3-carboxamide

[symbol]

### Step 1: ethyl 2-chloro-6-(5,6-dimethoxy-1H-benzo[d]imidazol-1-yl)nicotinate

[symbol]

To a stirred solution of 5,6-dimethoxy-1H-benzo[d]imidazole (1 g, 5.6 mmol, 1.0 equiv.) at 0° C. in DMF (20 mL) was added NaH (60% dispersion in mineral oil) (224 mg, 5.6 mmol, 1.0 equiv.), followed by DABCO (628 mg, 5.6 mmol, 1.0 equiv.). After stirring for 15 minutes at 0° C., ethyl 2,6-dichloronicotinate (1.226 g, 5.6 mmol, 1.0 equiv.) was added. Stirring at 0° C. was continued for 2 hours.

The mixture was poured into ice-cold H₂O (100 mL). The precipitated solid was collected by filtration, washed with H₂O and dried at 60° C. for 16 hours. The crude title compound (1.46 g, 72% yield) was obtained as a yellow solid. LC-MS: m/z=362 [M+H]+, ESI pos.

### Step 2: ethyl 2-(2-chlorophenoxy)-6-(5,6-dimethoxybenzimidazol-1-yl)pyridine-3-carboxylate

[symbol]

A stirred mixture of ethyl 2-chloro-6-(5,6-dimethoxy-1H-benzo[d]imidazol-1-yl)nicotinate (100 mg, 0.28 mmol, 1.0 equiv.), 2-chlorophenol (44 mg, 0.34 mmol, 1.2 equiv.) and Cs₂CO₃ (182 mg, 0.56 mmol, 2.0 equiv.) in DMF (2 mL) was heated at 50° C. for 2 hours. The reaction mixture was cooled to RT, poured into H₂O and extracted with EtOAc (3×). The combined organics were washed with H₂O (3 x) and brine, dried over MgSO₄, filtered and concentrated to dryness. The crude title compound (141 mg, quantitative yield) was obtained and was used in the next step without further purification.

### Step 3: 2-(2-chlorophenoxy)-6-(5,6-dimethoxybenzimidazol-1-yl)pyridine-3-carboxamide

[symbol]

A mixture of the crude ethyl 2-(2-chlorophenoxy)-6-(5,6-dimethoxybenzimidazol-1-yl)pyridine-3-carboxylate (141 mg, 0.28 mmol, 1.0 equiv.) and KOH (56 mg, 1 mmol, 3.6 equiv.) in a mixture of THF/CH₃CN/MeOH/H₂O 1:1:1:1 (4 mL) was stirred RT for 1 hour. AcOH (0.5 mL) was added to the mixture which was then concentrated in vacuo. The residue was triturated in a mixture of EtOAc and Et₂O. The solid was collected by filtration, dried and suspended in CH₂Cl₂ (10 mL). To the reaction mixture were added oxalyl chloride (250 μL) and 1 drop of DMF. After stirring for 1 hour at RT, the mixture was cooled to 0° C. and concentrated NH₄OH (2 mL) was added dropwise. The mixture was stirred at 0° C. for 30 minutes and then at RT for 1 hour. The mixture was filtered and the layers were separated. The aqueous phase was extracted with CH₂Cl₂ (2 x). All the combined organic layers were washed with brine, dried over Na₂SO₄, filtered and concentrated to dryness. The residue was purified by flash chromatography (SiO₂, 5% MeOH in CH₂Cl₂). The title compound (34 mg, 28.6% yield) was obtained as a white solid. LC-MS: m/z=425 [M+H]⁺, ESI pos.

### Example 2

### 6-(5,6-Dimethoxy-benzoimidazol-1-yl)-2-phenylamino-nicotinamide

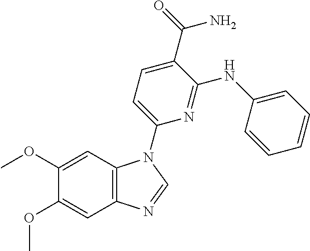 [SMILES: COc1cc2ncn(-c3ccc(C(N)=O)c(Nc4ccccc4)n3)c2cc1OC]

### Step 1: 2-chloro-6-(5,6-dimethoxybenzimidazol-1-yl)pyridine-3-carboxamide

[symbol]

A mixture of ethyl 2-chloro-6-(5,6-dimethoxy-1H-benzo[d]imidazol-1-yl)nicotinate (obtained as in step 1 of example 1) (250 mg, 0.69 mmol, 1.0 equiv.) and KOH (100 mg, 1.78 mmol, 2.6 equiv.) in a mixture of THF/CH₃CN/MeOH/H₂O 1:1:1:1 (4 mL) was stirred at RT for 2 hours. The reaction mixture was concentrated in vacuo. The residual solid was taken in CH₂Cl₂ (10 mL). Thionyl chloride (1 mL) and DMF (0.5 mL) were added and the mixture was stirred at reflux for 3 hours. The mixture was cooled to RT and concentrated to dryness. The residual solid was suspended in CH₂Cl₂ (10 mL) and concentrated NH₄OH (1 mL) was added dropwise at 0° C. After stirring for 1 hour at 0° C., the cooling bath was removed and the mixture was stirred overnight at RT. The mixture was partitioned between EtOAc (25 mL) and H₂O (25 mL). The insoluble materials were filtered off. The layers in the filtrate were separated. The aqueous phase was extracted with EtOAc (3 x). The combined organic extracts were dried over Na₂SO₄, filtered and concentrated in vacuo. The residue was triturated in a mixture of EtOAc/hexane to give a suspension. The solid was collected by filtration and dried to afford the crude title compound which was used without further purification.

### Step 2: 2-anilino-6-(5,6-dimethoxybenzimidazol-1-yl)pyridine-3-carboxamide

[symbol]

A mixture of the crude 2-chloro-6-(5,6-dimethoxybenzimidazol-1-yl)pyridine-3-carboxamide (75 mg, 0.23 mmol, 1.0 equiv.) and aniline (93 mg, 1 mmol, 1.0 equiv.) in ethylene glycol (1 mL) was stirred at 160° C. for 8 hours. The reaction mixture was poured into H₂O and extracted with EtOAc (3 x). The combined organic layers were washed with brine, dried over MgSO₄, filtered and concentrated. The residual dark oil was purified by flash chromatography (SiO2, 10% MeOH in CH₂Cl₂). The title compound (20 mg, 21.7% yield) was obtained as an orange foam. LC-MS: m/z=390 [M+H]+, ESI pos.

### Example 3

### 2-(benzylamino)-6-(5,6-dimethoxybenzimidazol-1-yl)pyridine-3-carboxamide

[symbol]

### Step 1: ethyl 2-(benzylamino)-6-chloro-pyridine-3-carboxylate

[symbol]

To a solution of ethyl 2,6-dichloronicotinate (3.3 g, 15 mmol, 1.0 equiv.) and NEt₃ (1.82 g, 18 mmol, 1.2 equiv.) in 2-methoxyethanol (30 mL) was added benzylamine (1.93 g, 18 mmol, 1.2 equiv.). The reaction mixture was heated to 80° C. and stirred for 15 hours. The mixture was cooled to RT and concentrated in vacuo. The residue was taken in EtOAc and washed with H₂O. The organic layer was dried over MgSO₄, filtered and concentrated tinder reduced pressure. The residue was purified by flash chromatography (SiO₂, 1% EtOAc in petroleum ether) to afford the title compound (3 g, 69% yield). ¹H NMR (CDCl₃, 300 MHz): δ 8.43 (br s, 1H), 8.05 (d, 1H, J=7.8 Hz), 7.39-7.26 (m, 5H), 6.54 (d, 1H, J=8.1 Hz), 4.73 (d, 2H, J=5.7 Hz), 4.31 (q, 2H, J=7.1 Hz), 1.37 (q, 3H, J=7.1 Hz).

### Step 2: ethyl 2-(benzylamino)-6-(5,6-dimethoxybenzimidazol-1-yl)pyridine-3-carboxylate

[symbol]

To a mixture of ethyl 2-(benzylamino)-6-chloro-pyridine-3-carboxylate (1 g, 3.45 mmol, 1.0 equiv.) and NaHCO₃ (0.35 g, 4.14 mmol, 1.2 equiv.) in DMSO (15 mL) was added 5,6-dimethoxy-1H-benzo[d]imidazole (0.74 g, 4.14 mmol, 1.2 equiv.). The reaction mixture was heated to 130° C. and stirring was continued for 20 hours. The mixture was cooled to RT and the solvent was removed under reduced pressure. The residue was diluted with H₂O and extracted with CH₂Cl₂. The organic layer was dried over MgSO₄, filtered and concentrated. The residue was purified by flash chromatography (SiO₂, 1% MeOH in CH₂Cl₂) to afford the title compound (0.83 g, 55% yield). LC-MS: m/z=433 [M+H]⁺, ESI pos.

### Step 3: 2-(benzylamino)-6-(5,6-dimethoxybenzimidazol-1-yl)pyridine-3-carboxylic acid

[symbol]

Starting with ethyl 2-(benzylamino)-6-(5,6-dimethoxybenzimidazol-1-yl)pyridine-3-carboxylate (0.11 g, 0.25 mmol, 1.0 equiv.) and following the procedure described in step 3 of example 6, the title compound (50 mg, 50% yield) was obtained.

### Step 4: 2-(benzylamino)-6-(5,6-dimethoxybenzimidazol-1-yl)pyridine-3-carboxamide

[symbol]

A mixture of 2-(benzylamino)-6-(5,6-dimethoxybenzimidazol-1-yl)pyridine-3-carboxylic acid (0.15 g, 0.37 mmol, 1.0 equiv.), EDCI (77 mg, 0.41 mmol, 1.1.0 equiv.) and HOBt (55 mg, 0.41 mmol, 1.1.0 equiv.) in DMF (6 mL) was heated to 50° C. and stirred for 1 hour. The reaction mixture was cooled to RT. Concentrated NH₄OH (1 mL) was added and the resulting solution was stirred at RT for 3 hours. The mixture was poured into H₂O resulting in a suspension. The precipitated solid was collected by filtration, washed with H₂O and dried. The title compound (105 mg, 70% yield) was obtained as an off-white solid. LC-MS: m/z=404 [M+H]+, ESI pos.

### Example 4

### 2-[[3-amino-1-(3-thienyl)propyl]amino]-6-(5,6-dimethoxybenzimidazol-1-yl)pyridine-3-carboxamide hydrochloride

[symbol]

### Step 1: ethyl 2-[[3-(tert-butoxycarbonylamino)-1-(3-thienyl)propyl]amino]-6-chloro-pyridine-3-carboxylate

[symbol]

Starting with tert-butyl N-[3-amino-3-(thiophen-3-yl)propyl]carbamate (prepared according to the procedure described in WO 2012/098068, p. 30) (1.23 g, 4.8 mmol, 1.2 equiv.) and according to the procedure described in step 1 of example 3, the title compound (0.75 g, 43% yield). LC-MS: m/z=440 [M+H]⁺, ESI pos.

### Step 2: ethyl 2-[[3-(tert-butoxycarbonylamino)-1-(3-thienyl)propyl]amino]-6-(5,6-dimethoxybenzimidazol-1-yl)pyridine-3-carboxylate

[symbol]

Starting with ethyl 2-[[3-(tert-butoxycarbonylamino)-1-(3-thienyl)propyl]amino]-6-chloro-pyridine-3-carboxylate (0.15 g, 0.34 mmol, 1.0 equiv.) and following the procedure described in step 2 of example 3, the title compound (0.16 g, 97.6% yield) was obtained. LC-MS: m/z=482 [M+H]⁺, ESI pos.

### Step 3: 2-[[3-amino-1-(3-thienyl)propyl]amino]-6-(5,6-dimethoxybenzimidazol-1-yl)pyridine-3-carboxylic acid hydrochloride

[symbol]

Starting with ethyl 2-[[3-(tert-butoxycarbonylamino)-1-(3-thienyl)propyl]amino]-6-(5,6-dimethoxybenzimidazol-1-yl)pyridine-3-carboxylate (0.11 g, 0.23 mmol, 1.0 equiv.) and following the procedure described in step 3 of example 3, the title compound (70 mg, 63% yield) was obtained. LC-MS: m/z=454 [M+H]⁺, ESI pos.

### Step 4: 2-[[3-amino-1-(3-thienyl)propyl]amino]-6-(5,6-dimethoxybenzimidazol-1-yl)pyridine-3-carboxamide hydrochloride

[symbol]

Starting with 2-[[3-amino-1-(3-thienyl)propyl]amino]-6-(5,6-dimethoxybenzimidazol-1-yl)pyridine-3-carboxylic acid hydrochloride (70 mg, 0.14 mmol, 1.0 equiv.) and following the procedure described in step 4 of example 3, the title compound (30 mg, 44% yield) was obtained as a white solid. LC-MS: m/z=453 [M+H]⁺, ESI pos.

### Example 5

### 2-[(3-amino-1-phenyl-propyl)amino]-6-(5,6-dimethoxybenzimidazol-1-yl)pyridine-3-carboxamide hydrochloride

[symbol]

### Step 1: ethyl 2-[[3-(tert-butoxycarbonylamino)-1-phenyl-propyl]amino]-6-chloro-pyridine-3-carboxylate

[symbol]

Starting with tert-butyl N-(3-amino-3-phenylpropyl)carbamate (prepared according to the procedure described in WO 2012/098068, p. 22) (0.9 g, 3.6 mmol, 1.2 equiv.) and according to the procedure described in step 1 of example 3, the title compound (0.27 g, 21% yield) was obtained LC-MS: m/z=434 [M+H]⁺, ESI pos.

### Step 2: ethyl 2-[(3-amino-1-phenyl-propyl)amino]-6-(5,6-dimethoxybenzimidazol-1-yl)pyridine-3-carboxylate hydrochloride

[symbol]

Starting with ethyl 2-[[3-(tert-butoxycarbonylamino)-1-phenyl-propyl]amino]-6-chloro-pyridine-3-carboxylate (40 mg, 0.092 mmol, 1.0 equiv.) and following the procedure described in step 2 of example 3, the title compound (35 mg, 74% yield) was obtained. LC-MS: m/z=476 [M+H]⁺, ESI pos.

### Step 3: 2-[(3-amino-1-phenyl-propyl)amino]-6-(5,6-dimethoxybenzimidazol-1-yl)pyridine-3-carboxylic acid

[symbol]

Starting with ethyl 2-[(3-amino-1-phenyl-propyl)amino]-6-(5,6-dimethoxybenzimidazol-1-yl)pyridine-3-carboxylate hydrochloride (190 mg, 0.37 mmol, 1.0 equiv.) and according to the procedure described in step 2 of example 17 the title compound (80 mg, 48.3% yield) was obtained. LC-MS: m/z=448 [M+H]⁺, ESI pos.

### Step 4: 2-[(3-amino-1-phenyl-propyl)amino]-6-(5,6-dimethoxybenzimidazol-1-yl)pyridine-3-carboxamide hydrochloride

[symbol]

Starting with 2-[(3-amino-1-phenyl-propyl)amino]-6-(5,6-dimethoxybenzimidazol-1-yl)pyridine-3-carboxylic acid (80 mg, 0.179 mmol, 1.0 equiv.) and following the procedure described in step 4 of example 3, the title compound (30 mg, 34.7% yield) was obtained as a white solid. LC-MS: m/z=447 [M+H]⁺, ESI pos.

### Example 6

6-(5,6-dimethoxybenzimidazol-1-yl)-2-(2-phenylethylamino)pyridine-3-carboxamide

[symbol]

### Step 1: ethyl 6-chloro-2-(2-phenylethylamino)pyridine-3-carboxylate

[symbol]

Starting with phenethylamine (0.58 g, 4.8 mmol, 1.2 equiv.) and according to the procedure described in step 1 of example 3, the title compound (0.9 g, 74% yield) was obtained. LC-MS: m/z=305 [M+H]⁺, ESI pos.

### Step 2: ethyl 6-(5,6-dimethoxybenzimidazol-1-yl)-2-(2-phenylethylamino)pyridine-3-carboxylate

[symbol]

Starting with ethyl 6-chloro-2-(2-phenylethylamino)pyridine-3-carboxylate (0.85 g, 2.8 mmol, 1.0 equiv.) and following the procedure described in step 2 of example 3, the title compound (0.68 g, 54% yield) was obtained. LC-MS: m/z=447 [M+H]⁺, ESI pos.

### Step 3: 6-(5,6-dimethoxybenzimidazol-1-yl)-2-(2-phenylethylamino)pyridine-3-carboxylic acid

[symbol]

To a solution of ethyl 6-(5,6-dimethoxybenzimidazol-1-yl)-2-(2-phenylethylamino)pyridine-3-carboxylate (0.22 g, 0.5 mmol, 1.0 equiv.) in a mixture of EtOH (10 mL) and H₂O (1 mL) was added KOH (0.7 g, 12.5 mmol, 25 equiv.). The reaction mixture was heated to reflux and stirred for 30 minutes. The mixture was cooled to RT and concentrated in vacuo. The residue was dissolved in H₂O and the aqueous phase was washed with CH₂Cl₂. The pH of the aqueous layer was adjusted to ˜4-5 with concentrated HCl, resulting in a suspension. The solid was collected by filtration and dried. The title compound (0.15 g, 72% yield) was obtained. LC-MS: m/z=419 [M+H]⁺, ESI pos.

### Step 4: 6-(5,6-dimethoxybenzimidazol-1-yl)-2-(2-phenylethylamino)pyridine-3-carboxamide

[symbol]

Starting with 6-(5,6-dimethoxybenzimidazol-1-yl)-2-(2-phenylethylamino)pyridine-3-carboxylic acid (70 mg, 0.167 mmol, 1.0 equiv.) and following the procedure described in step 4 of example 3, the title compound (38 mg, 54% yield) was obtained a an off-white solid. LC-MS: m/z=418 [M+H]⁺, ESI pos.

### Example 7

### 6-(5,6-dimethoxybenzimidazol-1-yl)-2-(2-thienylmethylamino)pyridine-3-carboxamide

[symbol]

### Step 1: ethyl 6-chloro-2-(2-thienylmethylamino)pyridine-3-carboxylate

[symbol]

Starting with 2-thiophenemethylamine (0.54 g, 4.8 mmol, 1.2 equiv.) and according to the procedure described in step 1 of example 3, the title compound (0.84 g, 71% yield) was obtained. LC-MS: m/z=297 [M+H]⁺, ESI pos.

### Step 2: ethyl 6-(5,6-dimethoxybenzimidazol-1-yl)-2-(2-thienylmethylamino)pyridine-3-carboxylate

[symbol]

Starting with ethyl 6-chloro-2-(2-thienylmethylamino)pyridine-3-carboxylate (0.8 g, 2.7 mmol, 1.0 equiv.) and following the procedure described in step 2 of example 3, the title compound (0.71 g, 58% yield) was obtained. LC-MS: m/z=439 [M+H]⁺, ESI pos.

### Step 3: 6-(5,6-dimethoxybenzimidazol-1-yl)-2-(2-thienylmethylamino)pyridine-3-carboxylic acid

[symbol]

Starting with ethyl 6-(5,6-dimethoxybenzimidazol-1-yl)-2-(2-thienylmethylamino)pyridine-3-carboxylate (0.35 g, 0.8 mmol, 1.0 equiv.) and according to the procedure described in step 3 of example 3, the title compound (275 mg, 84% yield) was obtained. LC-MS: m/z=411 [M+H]⁺, ESI pos.

### Step 4: 6-(5,6-dimethoxybenzimidazol-1-yl)-2-(2-thienylmethylamino)pyridine-3-carboxamide

[symbol]

Starting with 6-(5,6-dimethoxybenzimidazol-1-yl)-2-(2-thienylmethylamino)pyridine-3-carboxylic acid (150 mg, 0.36 mmol, 1.0 equiv.) and following the procedure described in step 4 of example 3, the title compound (135 mg, 90% yield) was obtained as a white solid. LC-MS: m/z=410 [M+H]⁺, ESI pos.

### Example 8

### 2-[(4-chlorophenyl)methylamino]-6-(5,6-dimethoxybenzimidazol-1-yl)pyridine-3-carboxamide

[symbol]

### Step 1: ethyl 6-chloro-2-[(4-chlorophenyl)methylamino]pyridine-3-carboxylate

[symbol]

Starting with 4-chlorobenzylamine (0.68 g, 4.8 mmol, 1.2 equiv.) and according to the procedure described in step 1 of example 3, the title compound (0.83 g, 64% yield) was obtained. LC-MS: m/z=325 [M+H]⁺, ESI pos.

### Step 2: ethyl 2-[(4-chlorophenyl)methylamino]-6-(5,6-dimethoxybenzimidazol-1-yl)pyridine-3-carboxylate

[symbol]

Starting with ethyl 6-chloro-2-[(4-chlorophenyl)methylamino]pyridine-3-carboxylate (0.8 g, 2.47 mmol, 1.0 equiv.) and following the procedure described in step 2 of example 3, the title compound (0.53 g, 46% yield) was obtained. LC-MS: m/z=467 [M+H]⁺, ESI pos.

### Step 3: 2-[(4-chlorophenyl)methylamino]-6-(5,6-dimethoxybenzimidazol-1-yl)pyridine-3-carboxylic acid

[symbol]

Starting with ethyl 2-[(4-chlorophenyl)methylamino]-6-(5,6-dimethoxybenzimidazol-1-yl)pyridine-3-carboxylate (0.2 g, 0.43 mmol, 1.0 equiv.) and according to the procedure described in step 3 of example 3, the title compound (155 mg, 82% yield) was obtained. LC-MS: m/z=439 [M+H]⁺, ESI pos.

### Step 4: 2-[(4-chlorophenyl)methylamino]-6-(5,6-dimethoxybenzimidazol-1-yl)pyridine-3-carboxamide

[symbol]

Starting with 6-(5,6-dimethoxybenzimidazol-1-yl)-2-(2-thienylmethylamino)pyridine-3-carboxylic acid (100 mg, 0.23 mmol, 1.0 equiv.) and following the procedure described in step 4 of example 3, the title compound (65 mg, 65% yield) was obtained as a white solid. LC-MS: m/z=438 [M+H]⁺, ESI pos.

### Example 9

2-[2-(3-chlorophenyl)ethylamino]-6-(5,6-dimethoxybenzimidazol-1-yl)pyridine-3-carboxamide

[symbol]

### Step 1: ethyl 6-chloro-2-[2-(3-chlorophenyl)ethylamino]pyridine-3-carboxylate

[symbol]

Starting with 2-(3-chlorophenyl)ethylamine (0.75 g, 4.8 mmol, 1.2 equiv.) and according to the procedure described in step 1 of example 3, the title compound (1.1 g, 68% yield) was obtained, ¹H NMR (CDCl₃, 300 MHz): δ 8.14 (br s, 1H), 8.01 (d, 1H, J=8.1 Hz), 7.25-7.12 (m, 4H), 6.51 (d, 1H, J=8.1 Hz), 4.33-4.26 (m, 2H), 3.75 (q, 2H, J=6.7 Hz), 2.92 (t, 2H, J=7.0 Hz), 1.36 (t, 3H, J=7.1 Hz).

### Step 2: ethyl 2-[2-(3-chlorophenyl)ethylamino]-6-(5,6-dimethoxybenzimidazol-1-yl)pyridine-3-carboxylate

[symbol]

Starting with ethyl 6-chloro-2-[2-(3-chlorophenyl)ethylamino]pyridine-3-carboxylate (1 g, 2.95 mmol, 1.0 equiv.) and following the procedure described in step 2 of example 3, the title compound (0.78 g, 50% yield) was obtained. LC-MS: m/z=481 [M+H]⁺, ESI pos.

### Step 3: 2-[2-(3-chlorophenyl)ethylamino]-6-(5,6-dimethoxybenzimidazol-1-yl)pyridine-3-carboxylic acid

[symbol]

Starting with ethyl 2-[2-(3-chlorophenyl)ethylamino]-6-(5,6-dimethoxybenzimidazol-1-yl)pyridine-3-carboxylate (0.2 g, 0.42 mmol, 1.0 equiv.) and according to the procedure described in step 3 of example 3, the title compound (160 mg, 84% yield) was obtained. LC-MS: m/z=453 [M+H]⁺, ESI pos.

### Step 4: 2-[2-(3-chlorophenyl)ethylamino]-6-(5,6-dimethoxybenzimidazol-1-yl)pyridine-3-carboxamide

[symbol]

Starting with 6-(5,6-dimethoxybenzimidazol-1-yl)-2-(2-thienylmethylamino)pyridine-3-carboxylic acid (120 mg, 0.265 mmol, 1.0 equiv.) and following the procedure described in step 4 of example 3, the title compound (105 mg, 87.5% yield) was obtained as a white solid. LC-MS: m/z=452 [M+H]⁺, ESI pos.

### Example 10

### 2-[[2-amino-1-(3-chlorophenyl)ethyl]amino]-6-(5,6-dimethoxybenzimidazol-1-yl)pyridine-3-carboxamide hydrochloride

[symbol]

### Step 1: ethyl 2-[[2-(tert-butoxycarbonylamino)-1-(3-chlorophenyl)ethyl]amino]-6-chloro-pyridine-3-carboxylate

[symbol]

Starting with tert-butyl N-[2-amino-2-(3-chlorophenyl)ethyl]carbamate (1.3 g, 4.8 mmol, 1.2 equiv,) and according to the procedure described in step 1 of example 3, the title compound (0.68 g, 37.6% yield) was obtained. ¹H NMR (CDCl₃, 300 MHz): δ 8.64 (d, 1H, J=7.8 Hz), 8.03 (d, 1H, J=8.1 Hz), 7.36-7.23 (m, 4H), 6.54 (d, 1H, J=7.8 Hz), 5.45-5.38 (m, 1H), 4.78-4.76 (m, 1H), 4.34 (q, 2H, J=7.0 Hz), 3.64-3.51 (m, 2H), 1.44-1.36 (m, 12H).

### Step 2: ethyl 2-[[2-amino-1-(3-chlorophenyl)ethyl]amino]-6-(5,6-dimethoxybenzimidazol-1-yl)pyridine-3-carboxylate hydrochloride

[symbol]

Starting with ethyl 2-[[2-(tert-butoxycarbonylamino)-1-(3-chlorophenyl)ethyl]amino]-6-chloro-pyridine-3-carboxylate (35 mg, 0.077 mmol, 1.0 equiv.) and following the procedure described in step 2 of example 3, the title compound (25 mg, 61% yield) was obtained. LC-MS: m/z=496 [M+H]⁺, ESI pos.

### Step 3: 2-[[2-amino-1-(3-chlorophenyl)ethyl]amino]-6-(5,6-dimethoxybenzimidazol-1-yl)pyridine-3-carboxylic acid hydrochloride

[symbol]

Starting with ethyl 2-[[2-amino-1-(3-chlorophenyl)ethyl]amino]-6-(5,6-dimethoxybenzimidazol-1-yl)pyridine-3-carboxylate hydrochloride (35 mg, 0.066 mmol, 1.0 equiv.) and according to the procedure described in step 3 of example 3, the crude title compound (43 mg) was obtained. LC-MS: m/z=468 [M+H]⁺, ESI pos.

### Step 4: 2-[[2-amino-1-(3-chlorophenyl)ethyl]amino]-6-(5,6-dimethoxybenzimidazol-1-yl)pyridine-3-carboxamide hydrochloride

[symbol]

Starting with the crude 2-[[2-amino-1-(3-chlorophenyl)ethyl]amino]-6-(5,6-dimethoxybenzimidazol-1-yl)pyridine-3-carboxylic acid hydrochloride (43 mg, 0.086 mmol, 1.0 equiv.) and following the procedure described in step 4 of example 3, the title compound (30 mg, 69% yield) was obtained as an off-white solid. LC-MS: m/z=467 [M+H]⁺, ESI pos.

### Example 11

### 2-[[3-amino-1-(3-chlorophenyl)propyl]amino]-6-(5,6-dimethoxybenzimidazol-1-yl)pyridine-3-carboxamide hydrochloride

[symbol]

### Step 1: ethyl 2-[[3-(tert-butoxycarbonylamino)-1-(3-chlorophenyl)propyl]amino]-6-chloro-pyridine-3-carboxylate

[symbol]

Starting with tert-butyl N-[3-amino-3-(3-chlorophenyl)propyl]carbamate (prepared according to the procedure described in WO 2012/098068, p. 24) (1.71 g, 6 mmol, 1.2 equiv.) and according to the procedure described in step 1 of example 3, the title compound (0.72 g, 31% yield) was obtained. LC-MS: m/z=468 [M+H]⁺, ESI pos.

### Step 2: ethyl 2-[[3-amino-1-(3-chlorophenyl)propyl]amino]-6-(5,6-dimethoxybenzimidazol-1-yl)pyridine-3-carboxylate

[symbol]

Starting with ethyl 2-[[3-(tert-butoxycarbonylamino)-1-(3-chlorophenyl)propyl]amino]-6-chloro-pyridine-3-carboxylate (80 mg, 0.17 mmol, 1.0 equiv.) and following the procedure described in step 2 of example 3, the title compound (52 mg, 59.6% yield) was obtained. LC-MS: m/z=510 [M+H]⁺, ESI pos.

### Step 3: 2-[[3-amino-1-(3-chlorophenyl)propyl]amino]-6-(5,6-dimethoxybenzimidazol-1-yl)pyridine-3-carboxylic acid hydrochloride

[symbol]

Starting with ethyl 2-[[3-amino-1-(3-chlorophenyl)propyl]amino]-6-(5,6-dimethoxybenzimidazol-1-yl)pyridine-3-carboxylate (0.13 g, 0.25 mmol, 1.0 equiv.) and according to the procedure described in step 3 of example 3, the crude title compound (130 mg, quantitative yield) was obtained. LC-MS: m/z=482 [M+H]⁺, ESI pos.

### Step 4: 2-[[3-amino-1-(3-chlorophenyl)propyl]amino]-6-(5,6-dimethoxybenzimidazol-1-yl)pyridine-3-carboxamide hydrochloride

[symbol]

Starting with the crude 2-[[3-amino-1-(3-chlorophenyl)propyl]amino]-6-(5,6-dimethoxybenzimidazol-1-yl)pyridine-3-carboxylic acid hydrochloride (130 mg, 0.25 mmol, 1.0 equiv.) and following the procedure described in step 4 of example 3, the title compound (62 mg, 48% yield) was obtained. LC-MS: m/z=481 [M+H]⁺, ESI pos.

### Example 12

### 2-[[2-amino-1-(3-thienyl)ethyl]amino]-6-(5,6-dimethoxybenzimidazol-1-yl)pyridine-3-carboxamide hydrochloride

[symbol]

### Step 1: ethyl 2-[[2-(tert-butoxycarbonylamino)-1-(3-thienyl)ethyl]amino]-6-chloro-pyridine-3-carboxylate

[symbol]

Starting with tert-butyl N-[2-amino-2-(thiophen-3-yl)ethyl]carbamate (prepared according to the procedure described in WO 2012/098068, p. 32) (1.16 g, 4.8 mmol, 1.2 equiv.) and according to the procedure described in step 1 of example 3, the title compound (405 ng, 24% yield) was obtained. ¹H NMR (CDCl₃, 300 MHz): δ 8.554 (d, 1H, J=8.4 Hz), 8.04 (d, 1H, J=8.1 Hz), 7.33-7.30 (m, 1H), 7.25 (br s, 1H), 7.13-7.12 (m, 1H), 6.54 (d, 1H, J=8.1 Hz), 5.62-5.56 (m, 1H), 4.86-4.85 (m, 1H), 4.32 (q, 2H, J=7.0 Hz), 3.67-3.57 (m, 2H), 1.39-1.34 (m, 12H).

### Step 2: ethyl 2-[[2-amino-1-(3-thienyl)ethyl]amino]-6-(5,6-dimethoxybenzimidazol-1-yl)pyridine-3-carboxylate hydrochloride

[symbol]

Starting with ethyl 2-[[2-(tert-butoxycarbonylamino)-1-(3-thienyl)ethyl]amino]-6-chloro-pyridine-3-carboxylate (35 mg, 0.082 mmol, 1.0 equiv.) and following the procedure described in step 2 of example 3, the title compound (40 mg, 97% yield) was obtained. LC-MS: m/z=468 [M+H]⁺, ESI pos.

### Step 3: 2-[[2-amino-1-(3-thienyl)ethyl]amino]-6-(5,6-dimethoxybenzimidazol-1-yl)pyridine-3-carboxylic acid hydrochloride

[symbol]

Starting with ethyl 2-[[2-amino-1-(3-thienyl)ethyl]amino]-6-(5,6-dimethoxybenzimidazol-1-yl)pyridine-3-carboxylate hydrochloride (0.048 g, 0.095 mmol, 1.0 equiv.) and according to the procedure described in step 3 of example 3, the crude title compound (0.053 g, quantitative yield) was obtained. LC-MS: m/z=440 [M+H]⁺, ESI pos.

### Step 4: 2-[[2-amino-1-(3-thienyl)ethyl]amino]-6-(5,6-dimethoxybenzimidazol-1-yl)pyridine-3-carboxamide hydrochloride

[symbol]

Starting with the crude 2-[[2-amino-1-(3-thienyl)ethyl]amino]-6-(5,6-dimethoxybenzimidazol-1-yl)pyridine-3-carboxylic acid hydrochloride (53 mg, 1.11 mmol, 1.0 equiv.) and following the procedure described in step 4 of example 3, the title compound (35 mg, 66.5% yield) was obtained as a white solid. LC-MS: m/z=439 [M+H]⁺, ESI pos.

### Example 13

### Methyl 2-(3-chlorophenyl)-6-(5,6-dimethoxy-1H-benzo[d]imidazol-1-yl)nicotinate

[symbol]

### Step 1: methyl 2-chloro-6-(5,6-dimethoxy-1H-benzo[d]imidazol-1-yl)nicotinate

[symbol]

In a 200 mL four-necked flask, NaH 60% dispersion in mineral oil (637 mg, 15.9 mmol, 2 equiv.) was combined with DMF (30 mL) to give a grey suspension. The mixture was cooled to 0° C. and 5,6-dimethoxy-1H-benzo[d]imidazole (1.42 g, 7.97 mmol, 1.0 equiv.) was added. The reaction mixture was stirred for 15 min. A solution of methyl 2,6-dichloronicotinate (1.64 g, 7.97 mmol, 1.0 equiv.) in DMF (10 mL) was added dropwise to the reaction mixture and stirring was continued for 15 min. The reaction mixture was quenched with 30 mL of 1 M HCl and a precipitate formed. The suspension was collected by filtration, washed with H₂O and dried under high vacuum. The title compound (1.414 g, 46.9% yield) was obtained as a light brown solid. LC-MS: m/z=348.1 [M+H]⁺, ESI pos.

### Step 2: methyl 2-(3-chlorophenyl)-6-(5,6-dimethoxy-1H-benzo[d]imidazol-1-yl)nicotinate

[symbol]

In a microwave vial were added methyl 2-chloro-6-(5,6-dimethoxy-1H-benzo[d]imidazol-1-yl)nicotinate (1 g, 2.88 mmol, 1.0 equiv.), 2 M Na₂CO₃ (5 mL, 10.1 mmol, 3.5 equiv.), 1,2-dimethoxyethane (15 mL), (3-chlorophenyl)boronic acid (674 mg, 4.31 mmol, 1.5 equiv.) and Pd(PPh₃)₄ (332 mg, 288 gmol, 0.1.0 equiv.). The vial was capped and heated in the microwave at 120° C. for 20 minutes. The reaction mixture was cooled to RT, diluted with 25 mL of H₂O and 25 mL of ethylacetate. The aqueous phase was back extracted with DCM. The combined organic layers were dried over Na₂SO₄, filtered and concentrated. The residue was purified by flash chromatography (silica gel, 80 g, 0% to 5% MeOH in DCM). The chromatographed product was triturated in acetone, collected by filtration, washed with acetone and dried. The title compound (353 mg, 27.2% yield) was obtained as a light brown solid. LC-MS: m/z=424.2 [M+H]⁺, ESI pos.

### Example 14

### Methyl 6-(5,6-dimethoxy-1H-benzo[d]imidazol-1-yl)-2-(2-oxopiperidin-1-yl)nicotinate

[symbol]

### Step 1: methyl 6-chloro-2-(2-oxopiperidin-1-yl)nicotinate

[symbol]

To a suspension of NaH 60% dispersion in mineral oil (208 mg, 5.2 mmol, 1.0 equiv.) in dry DMF (31 mL) was added piperidin-2-one (515 mg, 5.2 mmol, 1.0 equiv.) and the reaction mixture was stirred at 23° C. for 30 min. The reaction mixture was cooled to 0° C. and methyl 6-chloro-2-fluoronicotinate (986 mg, 5.2 mmol, 1.0 equiv.) was added. The cooling bath was removed and the solution was allowed to warm up to 23° C. Stirring was continued for 1 hours. The reaction mixture was added to ice-cold saturated aqueous NH₄Cl solution (100 mL), and extracted with EtOAc. The combined organics were washed with brine, dried over MgSO₄, filtered and concentrated to leave a light yellow liquid. This crude material was purified by flash chromatography (silicagel, 20 g, 0-80% heptane in EtOAc). The title compound (791 mg, 53.8% yield) was obtained as a light yellow solid. LC-MS: m/z=269.1 [M+H]⁺, ESI pos.

### Step 2: methyl 6-(5,6-dimethoxy-1H-benzo[d]imidazol-1-yl)-2-(2-oxopiperidin-1-yl)nicotinate

[symbol]

A suspension of sodium hydride (60% dispersion in mineral oil) (20 mg, 0.5 mmol, 1.0 equiv.) in dry DMF (1.9 ml) was cooled to 0° C. and 5,6-dimethoxy-1H-benzo[d]imidazole (89.1 mg, 0.5 mmol, 1.0 equiv.) was added and the reaction mixture was stirred for 15 min. Methyl 6-chloro-2-(2-oxopiperidin-1-yl)nicotinate. (134 mg, 0.5 mmol, 1.0 equiv.) was dissolved in dry DMF (600 μl)) and the solution was added dropwise. Stirring was continued for 15 min at 0° C. then let to warm overnight to RT. The reaction mixture was cooled, and diluted with cold sat. aq. NH₄Cl sol. and extracted with EtOAc. The combined organic layers were washed with brine, dried over MgSO₄ and concentrated under reduced pressure. The crude product was purified by flash column chromatography (silica gel, 0-10% MeOH in DCM) to yield the title compound (62.5 mg, 28.9% yield) was obtained as an off-white solid. LC-MS: m/z=411.2 [M+H]⁺, ESI pos.

### Example 15

### Methyl 6-(5,6-dimethoxy-1H-benzo[d]imidazol-1-yl)-2-(2-oxopyrrolidin-1-yl)nicotinate

[symbol]

### Step 1: methyl 6-chloro-2-(2-oxopyrrolidin-1-yl)nicotinate

[symbol]

Following the procedure described in step 1 of example 14 with pyrrolidin-2-one (443 mg, 5.2 mmol, 1.0 equiv.), the title compound (744 mg, 53.4% yield) was obtained as an orange liquid. LC-MS: m/z=255.1 [M+H]⁺, ESI pos.

### Step 2: methyl 6-(5,6-dimethoxy-1H-benzo[d]imidazol-1-yl)-2-(2-oxopyrrolidin-1-yl)nicotinate

[symbol]

Following the procedure described in step 2 of example 14 (with reaction time of 21 h at rt), the title compound (236 mg, 56.6% yield) was obtained as an off-white solid. LC-MS: m/z=397.2 [M+H]⁺, ESI pos.

### Example 16

### 6-(5,6-Dimethoxy-1H-benzo[d]imidazol-1-yl)-2-(piperidin-1-yl)nicotinic acid

[symbol]

To a stirred solution of methyl 6-(5,6-dimethoxy-1H-benzo[d]imidazol-1-yl)-2-(2-oxopiperidin-1-yl)nicotinate (obtained as in step 2 of example 14) (62 mg, 0.15 mmol, 1.0 equiv.) in a mixture of dry THF (560 μL) and ethanol (560 μL) was added calcium chloride (58 mg, 525 gmol, 3.5 equiv.). The reaction mixture was cooled to 0° C. NaBH₄ (25 mg, 675 gmol, 4.5 equiv.) was added in one portion and the mixture was stirred for 10 minutes. The cooling bath was removed and stirring at RT was continued for 2 hours. The mixture was poured into ice-cold sat. aq. NH₄Cl sol. (50 mL) and this was extracted with DCM. The organic layer was washed with brine, dried over MgSO₄, filtered and concentrated to dryness. The crude product was purified by prep. HPLC. The title compound (9.3 mg, 15.4% yield) was obtained as a white solid. LC-MS: m/z=381.2 [M−H]⁻, ESI neg.

### Example 17

### 2-(3-cyanophenyl)-6-(5,6-dimethoxybenzimidazol-1-yl)-N-ethyl-pyridine-3-carboxamide

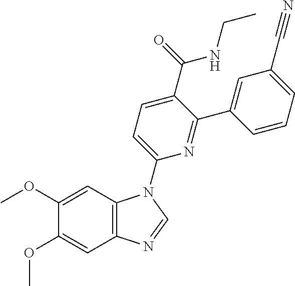 [SMILES: CCNC(=O)c1ccc(-n2cnc3cc(OC)c(OC)cc32)nc1-c1cccc(C#N)c1]

### Step 1: methyl 2-(3-cyanophenyl)-6-(5,6-dimethoxybenzimidazol-1-yl)pyridine-3-carboxylate

[symbol]

Methyl 2-chloro-6-(5,6-dimethoxybenzimidazol-1-yl)pyridine-3-carboxylate (obtained as in step 1 of example 1) (2.2 g, 6.33 mmol, 1.0 equiv.), 3-(4,4,5,5-tetramethyl-1,3,2-dioxaborolan-2-yl)benzonitrile (1.59 g, 6.96 mmol, 1.1.0 equiv.), PdCl₂(dppf) CH₂Cl₂ (517 mg, 0.630 mmol, 0.1.0 equiv.) and Na₂CO₃ (1.34 g, 12.65 mmol, 2 equiv.) were added to a mixture of 1,4-dioxane (20 mL) and H₂O (5 mL). The reaction mixture was heated to 80° C. and stirred for 12 hours. The mixture was cooled to RT, diluted with H₂O (100 mL) and EtOAc (100 mL) and stirred for 30 minutes. The insoluble materials were filtered off and the filter cake was washed with 100 mL of EtOAc. The organic layer from the filtrate was washed with brine, dried over anhydrous Na₂SO₄, filtered and concentrated to leave the crude title compound (2.2 g, 83.9% yield) as a dark brown solid. LC-MS: m/z=415.3 [M+H]⁺, ESI pos.

### Step 2: 2-(3-cyanophenyl)-6-(5,6-dimethoxybenzimidazol-1-yl)pyridine-3-carboxylic acid

[symbol]

To a solution of methyl 2-(3-cyanophenyl)-6-(5,6-dimethoxybenzimidazol-1-yl)pyridine-3-carboxylate (2.2 g, 5.31 mmol, 1.0 equiv.) in a mixture of THF (31 mL) and methanol (21 mL) was added lithium hydroxide hydrate 0.5 M in H₂O (21.2 mL, 10.62 mmol, 2 equiv.). The mixture was stirred at 20° C. for 12 hours, then at 30° C. for 4 hours. The pH of the dark reaction mixture was adjusted to 7 by the addition of 1 N HCl. The organic solvents were removed under reduced pressure and the residual aqueous solution was freeze-dried to afford the title compound (1.75 g, 72.4% yield) as a black solid.

LC-MS: m/z=401.0 [M+H]⁺, ESI pos.

### Step 3: 2-(3-cyanophenyl)-6-(5,6-dimethoxybenzimidazol-1-yl)-N-ethyl-pyridine-3-carboxamide

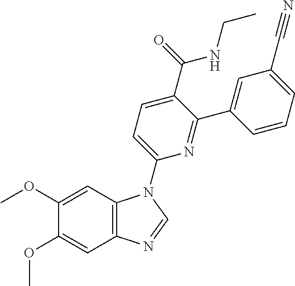 [SMILES: CCNC(=O)c1ccc(-n2cnc3cc(OC)c(OC)cc32)nc1-c1cccc(C#N)c1]

To a solution of 2-(3-cyanophenyl)-6-(5,6-dimethoxybenzimidazol-1-yl)pyridine-3-carboxylic acid (100 mg, 0.250 mmol, 1 eq) in DMF (2 mL) were added ethylamine (0.02 mL, 0.370 mmol, 1.5 eq), N,N-diisopropylethylamine (0.130 mL, 0.750 mmol, 3 eq) and HATU (140 mg, 0.370 mmol, 1.5 eq). The reaction mixture was stirred at 30° C. for 3 hours. The reaction mixture was filtered. The filtrate was purified by preparative HPLC (Waters Xbridge C18 (150 mm×50 mm×10 μm). Flow rate: 60 mL/min. Gradient: 35% to 85% CH3CN in (10 mM NH4HCO3 in water) then 100% CH₃CN (4 min)) to give give the title compound (25.5 mg, 0.060 mmol, 23.9% yield) as white solid. LC-MS: m/z=428.2 [M+H]+, ESI pos. 1H NMR (400 MHz, CDCl3) δ=8.45 (s, 1H), 8.20 (s, 1H), 8.16 (d, J=8.4 Hz, 1H), 8.07 (d, J=8.1 Hz, 1H), 7.93 (s, 1H), 7.80 (d, J=7.7 Hz, 1H), 7.66-7.58 (m, 2H), 7.33 (s, 1H), 5.61 (s, 1H), 3.97 (d, J=12.3 Hz, 6H), 3.44-3.35 (m, 2H), 2.02 (s, 1H), 1.09 (t, J=7.3 Hz, 3H).

### Example 18

### 2-(3-Cyanophenyl)-6-(5,6-dimethoxybenzimidazol-1-yl)-N-methyl-pyridine-3-carboxamide; Formic Acid

[symbol]

To a stirred solution of 2-(3-cyanophenyl)-6-(5,6-dimethoxybenzimidazol-1-yl)pyridine-3-carboxylic acid (obtained as in step 2 of example 17) (100 mg, 0.250 mmol, 1.0 equiv.) in DMF (2 mL) were added methylamine hydrochloride (0.04 mL, 0.5 mmol, 2 equiv.), N,N-diisopropylethylamine (0.13 mL, 0.75 mmol, 3.0 equiv.) and HATU (0.14 g, 0.37 mmol, 1.5 equiv.). The reaction mixture was stirred at 30° C. for 12 hours. The reaction mixture was directly purified using preparative HPLC: Column Phenomenex Synergi C18 150 mm×25 mm×10 μm). Flow rate 25 mL/min. Gradient: 21% to 41% CH₃CN in (H₂O with 0.225% formic acid v/v) (10 min) then 100% CH₃CN (2 min). The title compound (6.9 mg, 6.3% yield) was obtained as a white lyophilized solid. LC-MS: m/z=414.3 [M+H]⁺, ESI pos. ¹H NMR (DMSO-d6, 400 MHz): δ 9.00 (s, 1H), 8.51 (br d, 1H, J=4.5 Hz), 8.39 (s, 1H), 8.26 (s, 1H), 8.1-8.2 (m, 3H), 8.03 (d, 1H, J=8.2 Hz), 7.97 (d, 1H, J=7.8 Hz), 7.73 (t, 1H, J=7.8 Hz), 7.34 (s, 1H), 3.84 (d, 6H, J=6.5 Hz), 2.70 (d, 3H, J=4.5 Hz).

### Example 19

### 2-(3-Cyanophenyl)-N-cyclopropyl-6-(5,6-dimethoxybenzimidazol-1-yl)pyridine-3-carboxamide

[symbol]

Starting from 2-(3-cyanophenyl)-6-(5,6-dimethoxybenzimidazol-1-yl)pyridine-3-carboxylic acid (obtained as in step 2 of example 17) (100 mg, 0.25 mmol, 1.0 equiv.) with cyclopropylamine (0.03 mL, 0.37 mmol, 1.5 equiv.) and following the procedure described in example 18, the title compound (20 mg, 7.9% yield) was obtained as a white lyophilized solid after purification by preparative HPLC: (Column Waters Xbridge (150 mm×25 mm×5 μm). Flow rate 25 mL/min. Gradient: 20% to 50% CH₃CN in (H₂O with 0.05% ammonium hydroxide v/v) (10 min) then 100% CH₃CN (2 min)). LC-MS: m/z=440.3 [M+H]⁺, ESI pos. ¹H NMR (400 MHz, DMSO-d6): δ=9.00 (s, 1H), 8.60 (d, 1H, J=3.9 Hz), 8.20 (s, 1H), 8.1-8.2 (m, 1H), 8.0-8.1 (m, 3H), 7.98 (d, 1H, J=7.6 Hz), 7.7-7.8 (m, 1H), 7.34 (s, 1H), 3.84 (d, 6H, J=3.9 Hz), 2.73 (dt, 1H, J=3.7, 7.3 Hz), 0.6-0.7 (m, 2H), 0.3-0.4 (m, 2H).

### Example 20

### 2-(3-Cyanophenyl)-6-(5,6-dimethoxybenzimidazol-1-yl)-N-(2,2,2-trifluoroethyl)pyridine-3-carboxamide

[symbol]

Starting from 2-(3-cyanophenyl)-6-(5,6-dimethoxybenzimidazol-1-yl)pyridine-3-carboxylic acid (obtained as in step 2 of example 17) (100 mg, 0.25 mmol, 1.0 equiv.) with 2,2,2-trifluoroethylamine (0.06 mL, 0.5 mmol, 2 equiv.) and following the procedure described in example 18 (temperature of the reaction 50° C.), the title compound (36.5 mg, 28.6% yield) was obtained as a white solid after purification by preparative HPLC: Column Waters Xbridge (150 mm×25 mm×5 μm). Flow rate 25 mL/min. Gradient: 27% to 57% CH₃CN in (H₂O with 0.05% ammonium hydroxide v/v) (9.5 min) then 100% CH₃CN (2 min). LC-MS: m/z=482.2 [M+H]⁺, ESI pos. ¹H NMR (400 MHz, DMSO-d6): δ=9.28 (t, 1H, J=6.1 Hz), 9.02 (s, 1H), 8.1-8.2 (m, 4H), 8.05 (d, 1H, J=7.9 Hz), 7.97 (d, 1H, J=7.9 Hz), 7.71 (t, 1H, J=7.9 Hz), 7.34 (s, 1H), 4.04 (br dd, 2H, J=6.6, 9.4 Hz), 3.84 (d, 6H, J=2.0 Hz).

### Example 21

### 2-(3-Cyanophenyl)-6-(5,6-dimethoxybenzimidazol-1-yl)pyridine-3-carboxamide

[symbol]

### Step 1: 2-(3-cyanophenyl)-6-(5,6-dimethoxybenzimidazol-1-yl)pyridine-3-carbonyl chloride

[symbol]

A mixture of 2-(3-cyanophenyl)-6-(5,6-dimethoxybenzimidazol-1-yl)pyridine-3-carboxylic acid (obtained as in step 2 of example 17) (400 mg, 1 mmol, 1.0 equiv.) and thionyl chloride (20 mL) was stirred at 80° C. for 2 hours. The was yellow suspension was concentrated to dryness to give the crude title compound (400 mg, 76.5% yield) as a yellow solid, which was used in next step without purificaton.

LC-MS: m/z=415.2 [M+H]⁺, ESI pos.

### Step 2: 2-(3-cyanophenyl)-6-(5,6-dimethoxybenzimidazol-1-yl)pyridine-3-carboxamide

[symbol]

2-(3-cyanophenyl)-6-(5,6-dimethoxybenzimidazol-1-yl)pyridine-3-carbonyl chloride (100 mg, 0.24 mmol, 1.0 equiv.) was added to a stirred, cooled (0° C.) NH₃ (41 mg, 2.39 mmol, 10 equiv.) solution in THF (2 mL). The cooling bath was removed and the reaction mixture was stirred at 30° C. for 3 hours. The reaction mixture was concentrated to dryness and the residue was purified by preparative HPLC. Column Waters Xbridge (150 mm×25 mm×5 μm). Flow rate 25 mL/min. Gradient: 15% to 45% CH₃CN in (H₂O with 0.05% ammonium hydroxide v/v) (10 min) then 100% CH₃CN (2 min). The title compound (5.2 mg, 5.2% yield) was obtained as a white lyophilized solid. LC-MS: m/z=400.2 [M+H]⁺, ESI pos. ¹H NMR (400 MHz, DMSO-d6): δ=9.00 (s, 1H), 8.28 (t, 1H, J=1.5 Hz), 8.1-8.2 (m, 1H), 8.1-8.1 (m, 4H), 7.97 (td, 1H, J=1.3, 7.9 Hz), 7.7-7.8 (m, 2H), 7.33 (s, 1H), 3.84 (d, 6H, J=5.6 Hz).

### Example 22

### 2-(3-Cyano-5-methyl-pyrazol-1-yl)-6-[5-(2-morpholinoethoxy)benzimidazol-1-yl]pyridine-3-carboxamide

[symbol]

### Step 1: 4-[2-(4-nitrophenoxy)ethyl]morpholine

[symbol]

To a stirred solution of 4-nitrophenol (5.0 g, 35.94 mmol, 1.0 equiv.), 2-morpholinoethanol (5.89 g, 35.94 mmol, 1.0 equiv.) and triphenylphosphine (10.37 g, 39.54 mmol, 1.1.0 equiv.) in THF (80 mL) was added dropwise a solution of DEAD (6.89 g, 39.54 mmol, 1.1.0 equiv.) in THF (20 mL) at 0° C. under a nitrogen atmosphere. The mixture was then stirred at 30° C. for 16 hours. The reaction mixture was poured into H₂O (200 mL) and extracted with ethyl acetate (3×200 mL). The combined organic layers were dried over anhydrous Na₂SO₄, filtered and concentrated in vacuo. The residue was purified by flash chromatography (silicagel, 10% MeOH in EtOAc). The title compound (3.6 g, 39.7% yield) was obtained as an off-white solid. LC-MS: m/z=253.0 [M+H]⁺, ESI pos. ¹H NMR (400 MHz, CD₃OD): δ=8.25-8.18 (m, 2H), 7.14-7.08 (m, 2H), 4.27 (t, J=5.4 Hz, 2H), 3.75-3.68 (m, 4H), 2.85 (t, J=5.5 Hz, 2H), 2.64-2.56 (m, 4H).

### Step 2: 4-(2-morpholin-4-ylethoxy)aniline

[symbol]

To a solution of 4-[2-(4-nitrophenoxy)ethyl]morpholine (3.5 g, 13.87 mmol, 1.0 equiv.) in MeOH (40 mL) was added 10% Pd/C (0.67 g, 0.630 mmol, 0.050 equiv.). The mixture was stirred at 25° C. for 16 h under H₂ (15 psi). The mixture was filtered and the filtrate was concentrated in vacuo to afford the crude title compound (2.7 g, 87.5% yield) as light yellow solid. LC-MS: m/z=223.1 [M+H]⁺, ESI pos.

### Step 3: N-[4-(2-morpholinoethoxy)phenyl]acetamide

[symbol]

To a stirred solution of 4-(2-morpholin-4-ylethoxy)aniline (2.7 g, 12.15 mmol, 1.0 equiv.) and triethylamine (5.1 mL, 36.44 mmol, 3.0 equiv.) in DCM (30 mL) was added acetic anhydride (1.49 g, 14.58 mmol, 1.2 equiv.) at 0° C. Then the mixture was stirred at 25° C. for 16 hours under a nitrogen atmosphere. Then the mixture was poured into H₂O (100 mL) and extracted with EtOAc (3×100 mL). The combined organic layers were dried over anhydrous Na₂SO₄, filtered and concentrated in vacuo. The residue was purified by flash chromatography (silica gel, 50% to 100% EtOAc in petroleum ether). The title compound (2.2 g, 74% yield) was obtained as a white solid. LC-MS: m/z=265.1 [M+H]⁺, ESI pos. ¹H NMR (400 MHz, CDCl₃): δ=7.41-7.36 (m, 2H), 7.17 (br s, 1H), 6.90-6.83 (m, 2H), 4.10 (t, J=5.7 Hz, 2H), 3.77-3.73 (m, 4H), 2.81 (t, J=5.7 Hz, 2H), 2.62-2.57 (m, 4H), 2.16 (s, 3H).

### Step 4: N-[4-(2-morpholinoethoxy)-2-nitro-phenyl]acetamide

[symbol]

To a stirred solution of N-[4-(2-morpholinoethoxy)phenyl]acetamide (1.8 g, 6.81 mmol, 1.0 equiv.) in acetic anhydride (20 mL, 180.35 mmol, 26.48 equiv.) was added dropwise nitric acid (2.6 mL, 37.88 mmol, 5.56 equiv.) at 0° C. The cooling bath was removed and the mixture was stirred at 20° C. for another hour. The mixture was quenched by the careful addition of ice-cold H₂O (150 mL). The mixture was then basified by the addition of 1 N NaOH until to pH 9 was reached. This was extracted with EtOAc (4×50 mL). The combined organic extracts were dried over anhydrous Na₂SO₄, filtered and concentrated in vacuo to afford the crude title compound (1.8 g, 85.4% yield) as a red oil. LC-MS: m/z=310.1 [M+H]⁺, ESI pos.

### Step 5: 4-(2-morpholinoethoxy)-2-nitro-aniline

[symbol]

To a stirred solution of N-[4-(2-morpholinoethoxy)-2-nitro-phenyl]acetamide (1.8 g, 3.23 mmol, 1.0 equiv.) in a mixture of EtOH (15 mL) and H₂O (5 mL) was added potassium hydroxide (1.63 g, 29.1 mmol, 5 equiv.). The mixture was stirred at 80° C. for 4 hours. The reaction mixture was cooled to RT and poured into H₂O (100 mL). This was extracted with EtOAc (3×50 mL). The combined organic layers were dried over anhydrous Na₂SO₄, filtered and concentrated in vacuo to afford the crude title compound (1.2 g, 77.1% yield) as red oil. LC-MS: m/z=268.2 [M+H]⁺, ESI pos. ¹H NMR (400 MHz, CDCl₃): δ=7.58 (d, J=2.9 Hz, 1H), 7.12-7.07 (m, 1H), 6.76 (d, J=9.0 Hz, 1H), 4.09 (t, J=5.6 Hz, 2H), 3.77-3.74 (m, 4H), 2.80 (t, J=5.6 Hz, 2H), 2.61-2.57 (m, 4H).

### Step 6: methyl 2-chloro-6-[4-(2-morpholinoethoxy)-2-nitro-anilino]pyridine-3-carboxylate

[symbol]

To a solution of methyl 2,6-dichloronicotinate (400 mg, 1.94 mmol, 1.0 equiv.) and 4-(2-morpholinoethoxy)-2-nitro-aniline (519 mg, 1.94 mmol, 1.0 equiv.) in 1,4-dioxane (10 mL) were added Cs₂CO₃ (1.9 g, 5.82 mmol, 3.0 equiv.) and t-BuXphos-Pd-G3 (CAS #1447963-75-8) (154 mg, 0.19 mmol, 0.1.0 equiv.). The reaction mixture was sparged with N₂ before it was heated to 100° C. and stirring was continued for 2 hours. The mixture was cooled to RT, poured into H₂O (15 mL) and extracted with EtOAc (3×15 mL). The combined organic extracts were washed with brine (50 mL), dried over anhydrous Na₂SO₄, filtered and concentrated under reduced pressure. The residue was divied into 2 equal portions. The first half of the crude material was purified by preparative TLC (silica gel, 10% MeOH in DCM, UV detection) to afford the title compound (88 mg, 10.4% yield) as a red solid. The second half of the crude material was purified by preparative HPLC: column Waters Xbridge C18 (150 mm×50 mm×10 μm). Flow rate: 60 mL/min. Gradient: 35% to 85% CH₃CN in (10 mM NH₄HCO₃ in H₂O) then 100% CH₃CN (4 min). More of the title compound (78 mg, 9.2% yield) was obtained as a red solid. LC-MS: m/z=437.0 [M+H]⁺, ESI pos.

### Step 7: methyl 6-[2-amino-4-(2-morpholinoethoxy)anilino]-2-chloro-pyridine-3-carboxylate

[symbol]

To a solution of methyl 2-chloro-6-[4-(2-morpholinoethoxy)-2-nitro-anilino]pyridine-3-carboxylate (78.0 mg, 0.180 mmol, 1.0 equiv.) in EtOH (1.5 mL) was added Fe (49.85 mg, 0.89 mmol, 5.0 equiv.), NH₄Cl (95.5 mg, 1.8 mmol, 10 equiv.) and water (0.3 mL). The reaction mixture was stirred under nitrogen atmosphere at 60° C. for 12 hours. The reaction mixture was concentrated to dryness and 5 mL of 10% MeOH in DCM was added to the residue. The resulting suspension was stirred at 50° C. for 30 minutes. The mixture was cooled to RT and filtered. The filtrate was concentrated to leave the crude material which was purified by preparative TLC (silica gel, 10% MeOH in DCM, UV detection). The title compound (35 mg, 48.2% yield) was obtained as a yellow oil. LC-MS: m/z=407.0 [M+H]⁺, ESI pos.

### Step 8: methyl 2-chloro-6-[5-(2-morpholinoethoxy)benzimidazol-1-yl]pyridine-3-carboxylate

[symbol]

A solution of methyl 6-[2-amino-4-(2-morpholinoethoxy)anilino]-2-chloro-pyridine-3-carboxylate (50 mg, 0.12 mmol, 1.0 equiv.) in trimethyl orthoformate (260 mg, 2.46 mmol, 20 equiv.) was stirred at 120° C. for 20 hours. The reaction mixture was cooled to RT and concentrated in vacuo. The residue was purified by preparative TLC (silica gel, 80% EtOAc in petroleum ether, UV detection, Rf 0.5). The title compound (20 mg, 39% yield) was obtained as a light brown solid. LC-MS: m/z=407.1 [M+H]⁺, ESI pos.

### Step 9: methyl 2-(3-cyano-5-methyl-pyrazol-1-yl)-6-[5-(2-morpholinoethoxy)benzimidazol-1-yl]pyridine-3-carboxylate

[symbol]

To a solution of methyl 2-chloro-6-[5-(2-morpholinoethoxy)benzimidazol-1-yl]pyridine-3-carboxylate (15 mg, 0.04 mmol, 1.0 equiv.) in DMSO (1.5 mL) were added 5-methyl-1H-pyrazole-3-carbonitrile (4 mg, 0.04 mmol, 1.0 equiv.) and K₂CO₃ (10 mg, 0.07 mmol, 2 equiv.). The reaction mixture was stirred at 50° C. for 2 hours. The mixture was directly purified by preparative HPLC: column Phenomenex Gemini-NX C18 (75 mm×30 mm×3 μm). Flow rate: 25 mL/min. Gradient: 23% to 53% CH₃CN in (0.05% NH₄OH in H₂O v/v) (7 min) then 100% CH₃CN (2 min). The title compound (10 mg, 57% yield) was isolated as a colorless oil. LC-MS: m/z=488.1 [M+H]⁺, ESI pos. ¹H NMR (400 MHz, CDCl₃): δ ppm 2.46-2.55 (m, 3H) 3.00-3.20 (m, 2H) 3.46-3.59 (m, 2H) 3.60-3.71 (m, 2H) 3.84 (s, 3H) 3.97-4.09 (m, 2H) 4.27-4.41 (m, 2H) 4.65-4.77 (m, 2H) 6.69 (s, 1H) 7.05-7.15 (m, 1H) 7.36-7.45 (m, 1H) 7.86-7.96 (m, 1H) 8.00-8.09 (m, 1H) 8.49-8.58 (m, 1H) 8.90-9.01 (m, 1H).

### Step 10: 2-(3-cyano-5-methyl-pyrazol-1-yl)-N-[(2,4-dimethoxyphenyl)methyl]-6-[5-(2-morpholinoethoxy)benzimidazol-1-yl]pyridine-3-carboxamide

[symbol]

To a solution of methyl 2-(3-cyano-5-methyl-pyrazol-1-yl)-6-[5-(2-morpholinoethoxy)benzimidazol-1-yl]pyridine-3-carboxylate (20 mg, 0.04 mmol, 1.0 equiv.) and 2,4-dimethoxybenzylamine (0.01 mL, 0.08 mmol, 2 equiv.) in Toluene (2 mL) was added TBD (9 mg, 0.06 mmol, 1.5 equiv.). The mixture was stirred at 100° C. for 16 hours. The mixture was cooled to RT and concentrated. The residue was purified by preparative TLC (silica gel, 10% MeOH in DCM, Rf 0.45, UV detection). The title compound (9 mg, 35.2% yield) was obtained as a colorless oil. LC-MS: m/z=623.2 [M+H]⁺, ESI pos.

### Step 11: 2-(3-cyano-5-methyl-pyrazol-1-yl)-6-[5-(2-morpholinoethoxy)benzimidazol-1-yl]pyridine-3-carboxamide

[symbol]

To a solution of 2-(3-cyano-5-methyl-pyrazol-1-yl)-N-[(2,4-dimethoxyphenyl)methyl]-6-[5-(2-morpholinoethoxy)benzimidazol-1-yl]pyridine-3-carboxamide (9 mg, 0.014 mmol, 1.0 equiv.) in DCM (1 mL) was added TFA (1.0 mL) at 0° C. The cooling bath was removed and stirring at RT was continued for 24 hours. The mixture was concentrated in vacuo and the residue was purified by preparative HPLC: column Waters Xbridge C18 (150 mm×25 mm×5 μm). Flow rate: 25 mL/min. Gradient: 16% to 46% CH₃CN in (10 mM NH₄HCO₃ in H₂O) (9 min) then 100% CH₃CN (0.5 min). The title compound (1.5 mg, 18.7% yield) was obtained as a white lyophilized solid. LC-MS: m/z=473.2 [M+H]⁺, ESI pos. ¹H NMR (400 MHz, METHANOL-d4): δ ppm 2.54 (s, 3H) 3.16-3.24 (m, 2H) 3.84-4.00 (m, 4H) 4.44 (br s, 2H) 4.53-4.73 (m, 4H) 6.83 (d, J=0.75 Hz, 1H) 7.14-7.22 (m, 1H) 7.41 (d, J=2.50 Hz, 1H) 8.13 (d, J=8.38 Hz, 1H) 8.21 (d, J=9.01 Hz, 1H) 8.42 (d, J=8.38 Hz, 1H) 9.00 (s, 1H).

### Example 23

### 2-(2,2-Difluoro-1-methyl-ethoxy)-6-[5-[(6-methylpyridazin-3-yl)amino]benzimidazol-1-yl]pyridine-3-carboxamide

[symbol]

### Step 1: methyl 6-chloro-2-(2,2-difluoro-1-methyl-ethoxy)pyridine-3-carboxylate

[symbol]

To a solution of 1,1-difluoropropan-2-ol (2.027 g, 15.83 mmol, 1.2 equiv.) in THF (100 mL) were added Cs₂CO₃ (8.59 g, 26.38 mmol, 2 equiv.) and methyl 6-chloro-2-fluoro-pyridine-3-carboxylate (2.5 g, 13.19 mmol, 1.0 equiv.). The reaction mixture was stirred at 30° C. for 21 h before it was concentrated under reduced pressure. To the residue was added H₂O (100 mL) and this was extracted with EtOAc (3×100 mL). The combined organic layers were concentrated the residue was purified by flash chromatography (silica gel, 20% EtOAc in petroleum ether). The title compound (3.75 g, quantitative yield) was obtained as light yellow oil. LC-MS: m/z=266.0 [M+H]⁺, ESI pos.

### Step 2: 6-chloro-2-(2,2-difluoro-1-methyl-ethoxy)-N-[(2,4-dimethoxyphenyl)methyl]pyridine-3-carboxamide

[symbol]

To a solution of methyl 6-chloro-2-(2,2-difluoro-1-methyl-ethoxy)pyridine-3-carboxylate (3.4 g, 12.8 mmol, 1.0 equiv.) and 2,4-dimethoxybenzylamine (2.9 mL, 19.2 mmol, 1.5 equiv.) in THF (50 mL) was added TBD (2.14 g, 15.36 mmol, 1.2 equiv.). The reaction mixture was stirred at 30° C. for 16 hours before it was concentrated under reduced pressure. The residue was purified by flash chromatography (silica gel, 20% EtOAc in petroleum ether). The title compound (4.136 g, 67.7% yield) was obtained as white solid. LC-MS: m/z=401.1 [M+H]⁺, ESI pos.

### Step 3: 2-(2,2-difluoro-1-methyl-ethoxy)-N-[(2,4-dimethoxyphenyl)methyl]-6-[5-[(6-methylpyridazin-3-yl)amino]benzimidazol-1-yl]pyridine-3-carboxamide and 2-(2,2-difluoro-1-methyl-ethoxy)-N-[(2,4-dimethoxyphenyl)methyl]-6-[6-[(6-methylpyridazin-3-yl)amino]benzimidazol-1-yl]pyridine-3-carboxamide

[symbol]

Following the procedure described in step 2 of example 27, with a reaction time of 18 h at 90° C., the first title compound 2-(2,2-difluoro-1-methyl-ethoxy)-N-[(2,4-dimethoxyphenyl)methyl]-6-[5-[(6-methylpyridazin-3-yl)amino]benzimidazol-1-yl]pyridine-3-carboxamide (77 mg, 24.3% yield) was obtained as a grey solid. LC-MS: m/z=590.2 [M+H]⁺, ESI pos.

¹H NMR (400 MHz, CDCl₃): δ=8.80 (d, J=8.1 Hz, 1H), 8.57 (s, 1H), 8.06 (br t, J=5.4 Hz, 1H), 7.95 (d, J=8.8 Hz, 1H), 7.80 (d, J=1.9 Hz, 1H), 7.48 (dd, J=1.9, 8.8 Hz, 1H), 7.39-7.30 (m, 2H), 7.27-7.23 (m, 1H), 7.19-7.08 (m, 2H), 6.54-6.46 (m, 2H), 6.00 (br t, J=55.1 Hz, 1H), 5.75-5.64 (m, 1H), 4.62 (d, J=5.6 Hz, 2H), 3.95-3.87 (m, 3H), 3.83 (s, 3H), 2.62 (s, 3H), 1.84 (br s, 3H), 1.56 (d, J=6.5 Hz, 3H).

Purification by preparative HPLC: column Waters Xbridge (150 mm×25 mm×5 μm). Flow rate: 25 mL/min. Gradient: 35% to 60% CH₃CN in (0.05% ammonium hydroxide in H₂O v/v) (10 min) then 100% CH₃CN (2 min).

The second title compound 2-(2,2-difluoro-1-methyl-ethoxy)-N-[(2,4-dimethoxyphenyl)methyl]-6-[6-[(6-methylpyridazin-3-yl)amino]benzimidazol-1-yl]pyridine-3-carboxamide (80 mg, 23.4% yield) was also obtained as a grey solid. LC-MS: m/z=590.2 [M+H]⁺, ESI pos.

¹H NMR (400 MHz, CDCl₃): δ=8.68 (d, J=8.3 Hz, 1H), 8.65 (d, J=1.4 Hz, 1H), 8.38 (s, 1H), 8.04 (br t, J=5.4 Hz, 1H), 7.70 (d, J=8.6 Hz, 1H), 7.28-7.14 (m, 3H), 7.12-7.01 (m, 3H), 6.89 (d, J=9.0 Hz, 1H), 6.44-6.36 (m, 2H), 6.06 (br t, J=55.0 Hz, 1H), 5.81-5.69 (m, 1H), 4.53 (d, J=5.6 Hz, 2H), 3.79 (s, 3H), 3.74 (s, 3H), 2.52 (s, 3H), 1.40 (d, J=6.5 Hz, 3H).

### Step 4: 2-(2,2-difluoro-1-methyl-ethoxy)-6-[5-[(6-methylpyridazin-3-yl)amino]benzimidazol-1-yl]pyridine-3-carboxamide

[symbol]

A mixture of TFA (1.0 mL, 13.46 mmol, 103.08 equiv.) and 2-(2,2-difluoro-1-methyl-ethoxy)-N-[(2,4-dimethoxyphenyl)methyl]-6-[5-[(6-methylpyridazin-3-yl)amino]benzimidazol-1-yl]pyridine-3-carboxamide (77 mg, 0.13 mmol, 1.0 equiv.) was stirred at 50° C. for 2 hours. The reaction was cooled to RT, concentrated under reduced pressure and the residue was purified by preparative HPLC: column Phenomenex Luna C18 (150 mm×25 mm×10 μm). Flow rate: 25 mL/min. Gradient: 6% to 36% CH₃CN in (0.225% formic acid in H₂O v/v) (10 min) then 100% CH₃CN (2 min). The title compound (44.5 mg, 77.5% yield) was obtained as an orange lyophilized solid. LC-MS: m/z=440.1 [M+H]⁺, ESI pos. ¹H NMR (400 MHz, CD₃OD): δ=8.90 (s, 1H), 8.60 (d, J=8.2 Hz, 1H), 8.20 (d, J=1.7 Hz, 1H), 8.13 (d, J=8.9 Hz, 1H), 7.66-7.59 (m, 2H), 7.49 (d, J=9.2 Hz, 1H), 7.27 (d, J=9.2 Hz, 1H), 6.41-6.09 (m, 1H), 5.86-5.73 (m, 1H), 2.57 (s, 3H), 1.60 (d, J=6.6 Hz, 3H).

### Example 24

### 2-(2,2-difluoro-1-methyl-ethoxy)-N-[(2,4-dimethoxyphenyl)methyl]-6-[6-[(6-methylpyridazin-3-yl)amino]benzimidazol-1-yl]pyridine-3-carboxamide

[symbol]

Starting with 2-(2,2-difluoro-1-methyl-ethoxy)-N-[(2,4-dimethoxyphenyl)methyl]-6-[6-[(6-methylpyridazin-3-yl)amino]benzimidazol-1-yl]pyridine-3-carboxamide (80 mg, 0.14 mmol, 1.0 equiv.), and following the procedure described in step 4 of example 23, the title compound (25.3 mg, 40.3% yield) was obtained as an orange lyophilized solid after purification by preparative HPLC: column Phenomenex Luna C18 (150 mm×25 mm×10 μm). Flow rate: 25 mL/min. Gradient: 6% to 36% CH₃CN in (0.225% formic acid in H₂O v/v) (10 min) then 100% CH₃CN (2 min). LC-MS: m/z=440.1 [M+H]⁺, ESI pos. ¹H NMR (400 MHz, METHANOL-d4): δ=9.18 (d, J=1.9 Hz, 1H), 8.79 (s, 1H), 8.62 (d, J=8.1 Hz, 1H), 7.63 (dd, J=8.4, 16.3 Hz, 2H), 7.37 (d, J=9.1 Hz, 1H), 7.25 (dd, J=1.9, 8.7 Hz, 1H), 7.13 (d, J=9.1 Hz, 1H), 6.45-6.13 (m, 1H), 5.89 (ddt, J=2.0, 6.4, 12.3 Hz, 1H), 2.54 (s, 3H), 1.48 (d, J=6.5 Hz, 3H).

### Example 25

### 2-(3-Cyano-5-methyl-pyrazol-1-yl)-6-[5-[[(3S,4R)-4-fluoropyrrolidin-3-yl]amino]benzimidazol-1-yl]pyridine-3-carboxamide; formic acid

[symbol]

### Step 1: methyl 6-chloro-2-(3-cyano-5-methyl-pyrazol-1-yl)pyridine-3-carboxylate

[symbol]

A solution of methyl 6-chloro-2-fluoro-pyridine-3-carboxylate (1.0 g, 5.28 mmol, 1.0 equiv.), 5-methyl-1H-pyrazole-3-carbonitrile (509 mg, 4.75 mmol, 0.9 equiv.) and DIPEA (2.6 mL, 15.83 mmol, 3.0 equiv.) in DMSO (15 mL) was stirred at 30° C. for 16 hours. The reaction mixture was concentrated under vacuum. The residue was purified by preparative HPLC: column Waters Xbridge (150 mm×25 mm×5 μm). Flow rate: 25 mL/min. Gradient: 41% to 71% CH₃CN in (0.05% NH₄OH in H₂O v/v) (10 min) then 100% CH₃CN (2 min). The title compound (700 mg, 48% yield) was obtained as white solid. LC-MS: m/z=276.9 [M+H]⁺, ESI pos. 1H NMR (400 MHz, CDCl₃): δ=8.20 (d, J=8.1 Hz, 1H), 7.54 (d, J=8.1 Hz, 1H), 6.60 (s, 1H), 3.79 (s, 3H), 2.51 (s, 3H).

### Step 2: methyl 6-(5-bromobenzimidazol-1-yl)-2-(3-cyano-5-methyl-pyrazol-1-yl)pyridine-3-carboxylate

[symbol]

To a solution of methyl 6-chloro-2-(3-cyano-5-methyl-pyrazol-1-yl)pyridine-3-carboxylate (700 mg, 2.53 mmol, 1.0 equiv.) in DMSO (7 mL) were added 5-bromo-1H-benzimidazole (499 mg, 2.53 mmol, 1.0 equiv.) and K₂CO₃ (699 mg, 5.06 mmol, 2 equiv.). The reaction mixture was stirred at 30° C. for 2 hours, and filtered. The filtrate was concentrated under reduced pressure and the residue was purified by preparative HPLC: column Phenomenex Luna C18 (150 mm×25 mm×10 μm). Flow rate: 25 mL/min. Gradient: 53% to 83% CH₃CN in (0.225% formic acid in H₂O v/v) (10 min) then 100% CH₃CN (2 min). The title compound (200 mg, 18.1% yield) was obtained as a white lyophilized solid. LC-MS: m/z=439.0 [M+H]⁺, ESI pos.

¹H NMR (400 MHz, CDCl₃): δ=9.17 (br s, 1H), 8.56 (d, J=8.2 Hz, 1H), 8.10 (d, J=1.5 Hz, 1H), 8.03 (d, J=8.8 Hz, 1H), 7.98 (br d, J=8.4 Hz, 1H), 7.59 (dd, J=1.7, 8.8 Hz, 1H), 6.68 (s, 1H), 3.83 (s, 3H), 2.51 (s, 3H).

### Step 3: methyl 2-(3-cyano-5-methyl-pyrazol-1-yl)-6-[5-[[(3S,4R)-1-tert-butoxycarbonyl-4-fluoro-pyrrolidin-3-yl]amino]benzimidazol-1-yl]pyridine-3-carboxylate

[symbol]

To a solution of methyl 6-(5-bromobenzimidazol-1-yl)-2-(3-cyano-5-methyl-pyrazol-1-yl)pyridine-3-carboxylate (190.0 mg, 0.430 mmol, 1.0 equiv.) in 1,4-dioxane (6 mL) was added tert-butyl (3S,4R)-3-amino-4-fluoropyrrolidine-1-carboxylate (106 mg, 0.52 mmol, 1.2 equiv.), Cs₂CO₃ (424.7 mg, 1.3 mmol, 3.0 equiv.) and t-Buxphos-Pd-G3 (34.5 mg, 0.040 mmol, 0.10 euiv.q). Then the grey suspension was stirred at 100° C. for 2 hours under nitrogen atmosphere. The reaction mixture was concentrated under reduced pressure to give the residue. After purification by preparative TLC (silica gel, 10% MeOH in DCM, UV detection), the title compound (70 mg, 28.8% yield) was obtained as a yellow oil. LC-MS: m/z=561.3 [M+H]⁺, ESI pos.

### Step 4: tert-butyl (3S,4R)-3-[[1-[6-(3-cyano-5-methyl-pyrazol-1-yl)-5-[(2,4-dimethoxyphenyl)methylcarbamoyl]-2-pyridyl]benzimidazol-5-yl]amino]-4-fluoro-pyrrolidine-1-carboxylate

[symbol]

Following the procedure described in step 2 of example 23, with a reaction time of 12 h at 50° C., and after purification by preparative TLC (silica gel, 10% MeOH in DCM, UV detection), the title compound (40 mg, 64.5% yield) was obtained as a yellow oil. LC-MS: m/z=696.3 [M+H]⁺, ESI pos.

### Step 5: 2-(3-cyano-5-methyl-pyrazol-1-yl)-6-[5-[[(3S,4R)-4-fluoropyrrolidin-3-yl]amino]benzimidazol-1-yl]pyridine-3-carboxamide; formic acid

[symbol]

A solution of tert-butyl (3S,4R)-3-[[1-[6-(3-cyano-5-methyl-pyrazol-1-yl)-5-[(2,4-dimethoxyphenyl)methylcarbamoyl]-2-pyridyl]benzimidazol-5-yl]amino]-4-fluoro-pyrrolidine-1-carboxylate (30 mg, 0.04 mmol, 1.0 equiv.) in TFA (0.5 mL) was stirred at 70° C. for 2 hours. The reaction mixture was concentrated under reduced pressure and the residue was purified by preparative HPLC: column Phenomenex Luna C18 (150 mm×25 mm×10 μm). Flow rate: 25 mL/min. Gradient: 1% to 30% CH₃CN in (0.225% formic acid in H₂O v/v) (10 min) then 100% CH₃CN (2 min). The title compound (15.6 mg, 69.4% yield) was obtained as a white lyophilized solid. LC-MS: m/z=446.1 [M+H]⁺, ESI pos.

¹H NMR (400 MHz, METHANOL-d4): δ=8.88 (s, 1H), 8.49 (br s, 1H), 8.37 (d, J=8.5 Hz, 1H), 8.07 (br d, J=8.4 Hz, 1H), 8.05 (br d, J=9.0 Hz, 1H), 7.09 (d, J=2.3 Hz, 1H), 6.95 (dd, J=2.3, 9.0 Hz, 1H), 6.81 (s, 1H), 5.28 (br s, 1H), 4.48-4.30 (m, 1H), 3.75-3.69 (m, 1H), 3.66 (s, 1H), 3.59 (br d, J=5.9 Hz, 1H), 3.15 (t, J=11.0 Hz, 1H), 2.52 (s, 3H).

### Example 26

### Methyl 2-(3-methoxy-5-methyl-pyrazol-1-yl)-6-[5-[(6-methylpyridazin-3-yl)amino]benzimidazol-1-yl]pyridine-3-carboxylate

[symbol]

### Step 1: methyl 6-chloro-2-(3-methoxy-5-methyl-pyrazol-1-yl)pyridine-3-carboxylate

[symbol]

Prepared in analogy to example 27, step 1 using methyl 6-chloro-2-fluoro-pyridine-3-carboxylate (1.0 g, 5.28 mmol, 1.0 equiv.,) and 3-methoxy-5-methyl-1H-pyrazole (600.0 mg, 5.35 mmol, 1.0 equiv.) to give methyl 6-chloro-2-(3-methoxy-5-methyl-pyrazol-1-yl)pyridine-3-carboxylate (1.4 g, 4.97 mmol, 94.2% yield) as white solid. LC-MS: m/z=282.2 [M+H]⁺, ESI pos.

### Step 2: methyl 2-(3-methoxy-5-methyl-pyrazol-1-yl)-6-[5-[(6-methylpyridazin-3-yl)amino]benzimidazol-1-yl]pyridine-3-carboxylate and methyl 2-(3-methoxy-5-methyl-pyrazol-1-yl)-6-[6-[(6-methylpyridazin-3-yl)amino]benzimidazol-1-yl]pyridine-3-carboxylate

[symbol]

Prepared in analogy to example 27, step 2 using of N-(6-methylpyridazin-3-yl)-1H-benzimidazol-5-amine (0.92 g, 4.07 mmol, 1.04 equiv., prepared in example 27, intermediate 1), methyl 6-chloro-2-(3-methoxy-5-methyl-pyrazol-1-yl)pyridine-3-carboxylate (1.1 g, 3.91 mmol, 1.0 equiv.) and K₂CO₃ (1.65 g, 11.94 mmol, 3.1.0 equiv.) to yield methyl 2-(3-methoxy-5-methyl-pyrazol-1-yl)-6-[6-[(6-methylpyridazin-3-yl)amino]benzimidazol-1-yl]pyridine-3-carboxylate (370 mg, 0.79 mmol, 20.1% yield) and methyl 2-(3-methoxy-5-methyl-pyrazol-1-yl)-6-[5-[(6-methylpyridazin-3-yl)amino]benzimidazol-1-yl]pyridine-3-carboxylate (350 mg, 0.74 mmol, 18.1% yield) as light yellow solids. LC-MS: m/z=471.1 [M+H]⁺, ESI pos.

### Example 27

### 1-[3-Acetyl-6-[5-[(6-methylpyridazin-3-yl)amino]benzimidazol-1-yl]-2-pyridyl]-5-methyl-pyrazole-3-carbonitrile

[symbol]

### Intermediate 1: N-(6-methylpyridazin-3-yl)-1H-benzimidazol-5-amine

Error! Objects cannot be created from editing field codes. A mixture of 5-aminobenzimidazole (8.0 g, 60.1 mmol, 1.0 equiv.) and 3-chloro-6-methylpyridazine (7.34 g, 57.08 mmol, 0.950 equiv.) in ⁱPrOH (120 mL) was stirred at 120° C. for 72 hours. The dark brown suspension was concentrated in vacuo and the residue was triturated in MeOH (60 mL). The solid was collected by filtration and it was triturated in DCM (40 mL). The product was collected by filtration, washed with DCM and dried. The title compound (10 g, 71.3% yield) was obtained as brown solid. LC-MS: m/z=226.0 [M+H]⁺, ESI pos. ¹H NMR (400 MHz, DMSO-d6): δ=9.60 (br s, 1H), 8.72 (s, 1H), 8.52 (d, J=1.7 Hz, 1H), 7.63 (d, J=8.8 Hz, 1H), 7.44 (dd, J=2.0, 8.8 Hz, 1H), 7.39 (d, J=9.2 Hz, 1H), 7.21 (d, J=9.2 Hz, 1H), 5.04-4.15 (m, 1H), 2.49 (s, 3H).

### Step 1: 1-(3-acetyl-6-chloro-2-pyridyl)-5-methyl-pyrazole-3-carbonitrile

[symbol]

A solution of 1-(6-chloro-2-fluoro-3-pyridyl)ethanone (5 g, 28.81 mmol, 1.0 equiv.), 5-methyl-1H-pyrazole-3-carbonitrile (2.93 g, 27.37 mmol, 0.950 equiv.) and DIPEA (14.3 mL, 86.42 mmol, 3 equiv.) in DMSO (50 mL) was stirred at 80° C. for 4 hours. The reaction mixture was cooled to RT, poured into H₂O (250 mL) and extracted with EtOAc (3×150 mL). The combined organic layers were washed with brine (3×300 mL) and concentrated in vacuo. The residue was purified by flash chromatography (silica gel, 0% to 35% EtOAc in petroleum ether). The title compound (5.3 g, 70.6% yield) was obtained as yellow oil. LC-MS: m/z=261.1 [M+H]⁺, ESI pos.

### Step 2: 1-[3-acetyl-6-[5-[(6-methylpyridazin-3-yl)amino]benzimidazol-1-yl]-2-pyridyl]-5-methyl-pyrazole-3-carbonitrile and 1-[3-acetyl-6-[6-[(6-methylpyridazin-3-yl)amino]benzimidazol-1-yl]-2-pyridyl]-5-methyl-pyrazole-3-carbonitrile

[symbol]

A mixture of 1-(3-acetyl-6-chloro-2-pyridyl)-5-methyl-pyrazole-3-carbonitrile (5.3 g, 20.33 mmol, 1.0 equiv.), N-(6-methylpyridazin-3-yl)-1H-benzimidazol-5-amine (intermediate 1) (4.58 g, 20.33 mmol, 1.0 equiv.) and K₂CO₃ (5.62 g, 40.66 mmol, 2 equiv.) in DMSO (50 mL) was stirred at 50° C. for 12 hours. The mixture was cooled to RT and poured into H₂O (500 mL). A solid precipitated out. This was extracted with EtOAc (3×400 mL). The combined organic layers were concentrated. The residue was purified by preparative HPLC: column Phenomenex Luna C18 (250 mm×70 mm×15 μm. Flow rate 140 mL/min. Gradient: 20% to 50% CH₃CN in (H₂O with 0.225% formic acid v/v) (35 min) then 100% CH₃CN (1 min). A mixture of the 2 title compounds was obtained. This mixture was purified by preparative NPLC: column Welch Ultimate XB-SiOH (250 mm×70 mm×10 um). Flow rate 140 mL/min. Gradient: 20% to 60% EtOH in hexane (20 min) then 100% EtOH (3 min).

The first title compound 1-[3-acetyl-6-[6-[(6-methylpyridazin-3-yl)amino]benzimidazol-1-yl]-2-pyridyl]-5-methyl-pyrazole-3-carbonitrile (1100 mg, 12% yield) was obtained as a light brown solid. LC-MS: m/z=450.1 [M+H]⁺, ESI pos. 1H NMR (400 MHz, DMSO-d₆) δ=9.31 (s, 1H), 9.01 (s, 1H), 8.98 (d, J=2.0 Hz, 1H), 8.55 (d, J=8.4 Hz, 1H), 8.27 (d, J=8.4 Hz, 1H), 7.70 (d, J=8.7 Hz, 1H), 7.46 (dd, J=2.0, 8.7 Hz, 1H), 7.30 (d, J=9.0 Hz, 1H), 7.09-7.04 (m, 2H), 2.55 (s, 3H), 2.50 (br s, 3H), 2.19 (s, 3H).

The second title compound 1-[3-acetyl-6-[5-[(6-methylpyridazin-3-yl)amino]benzimidazol-1-yl]-2-pyridyl]-5-methyl-pyrazole-3-carbonitrile (600 mg, 6.6% yield) was obtained as light brown solid. LC-MS: m/z=450.1 [M+H]⁺, ESI pos. ¹H NMR (400 MHz, DMSO-d₆) δ=9.30 (s, 1H), 9.11 (s, 1H), 8.54 (d, J=8.4 Hz, 1H), 8.47 (d, J=1.7 Hz, 1H), 8.30 (d, J=8.6 Hz, 1H), 8.14 (d, J=8.9 Hz, 1H), 7.53 (dd, J=1.9, 8.9 Hz, 1H), 7.34 (d, J=9.0 Hz, 1H), 7.14 (s, 1H), 7.10 (d, J=9.0 Hz, 1H), 2.54 (s, 3H), 2.48 (br s, 3H), 2.20 (s, 3H).

### Example 28

### 1-[3-Acetyl-6-[6-[(6-methylpyridazin-3-yl)amino]benzimidazol-1-yl]-2-pyridyl]-5-methyl-pyrazole-3-carbonitrile

[symbol]

1-[3-acetyl-6-[6-[(6-methylpyridazin-3-yl)amino]benzimidazol-1-yl]-2-pyridyl]-5-methyl-pyrazole-3-carbonitrile has been obtained in example 27, step 2. LC-MS: m/z=450.2 [M+H]⁺, ESI pos.

### Example 29

### 1-[2,4-dimethoxy-6-[5-[(6-methylpyridazin-3-yl)amino]benzimidazol-1-yl]-3-pyridyl]ethanone

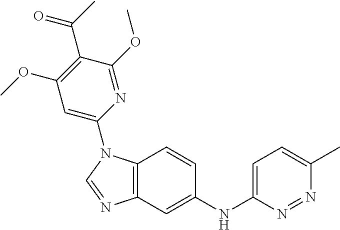

1-[2,4-dimethoxy-6-[5-[(6-methylpyridazin-3-yl)amino]benzimidazol-1-yl]-3-pyridyl]ethanone was made according to methods described herein and methods known to those skilled in the art. LC-MS: m/z=405.2 [M+H]⁺, ESI pos.

### Example 30

### 1-[3-acetyl-6-(6,7-dihydro-5H-pyrrolo[3,2-f]benzimidazol-3-yl)-2-pyridyl]-5-methyl-pyrazole-3-carbonitrile

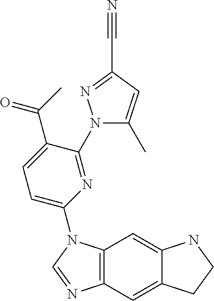 [SMILES: CC(=O)c1ccc(-n2cnc3cc4c(cc32)NCC4)nc1-n1nc(C#N)cc1C]

1-[3-acetyl-6-(6,7-dihydro-5H-pyrrolo[3,2-f]benzimidazol-3-yl)-2-pyridyl]-5-methyl-pyrazole-3-carbonitrile was synthesized according to the methods described herein, using intermediate 18 and 3,5,6,7-tetrahydropyrrolo[3,2-f]benzimidazole (CAS: 28996-22-7). LC-MS: m/z=384.2=[M+H]⁺, ESI pos.

### Example 31

### 1-[3-acetyl-6-(6,7-dihydro-5H-pyrrolo[2,3-f]benzimidazol-1-yl)-2-pyridyl]-5-methyl-pyrazole-3-carbonitrile

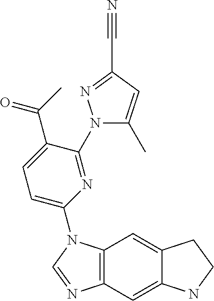 [SMILES: CC(=O)c1ccc(-n2cnc3cc4c(cc32)CCN4)nc1-n1nc(C#N)cc1C]

1-[3-acetyl-6-(6,7-dihydro-5H-pyrrolo[3,2-f]benzimidazol-3-yl)-2-pyridyl]-5-methyl-pyrazole-3-carbonitrile was synthesized according to the methods described herein, using intermediate 18 and 3,5,6,7-tetrahydropyrrolo[3,2-f]benzimidazole (CAS: 28996-22-7). LC-MS: m/z=384.2=[M+H]⁺, ESI pos.

### Example 32

### (3R,5S)-1-[3-formyl-6-[6-[(6-methylpyridazin-3-yl)amino]benzimidazol-1-yl]-2-pyridyl]-5-methyl-pyrrolidine-3-carbonitrile

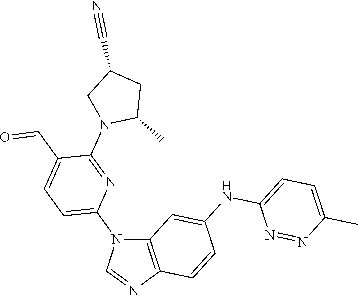 [SMILES: Cc1ccc(Nc2ccc3ncn(-c4ccc(C=O)c(N5C[C@H](C#N)C[C@@H]5C)n4)c3c2)nn1]

### Step 1: tert-butyl (2S,4S)-2-methyl-4-methylsulfonyloxy-pyrrolidine-1-carboxylate

[symbol]

To a solution of tert-butyl (2R,4R)-4-hydroxy-2-methyl-pyrrolidine-1-carboxylate (1.0 g, 4.97 mmol, 1.0 equiv.) and TEA (2.5 g, 24.9 mmol, 5.0 equiv.) in DCM (10 mL) was added MsCl (0.78 mL, 9.95 mmol, 2.0 equiv.) dropwise at 0° C. The reaction mixture was stirred at 0° C. for 3 hours. TLC (PE/EA=1/1, ninhydrin) showed tert-butyl (2R,4R)-4-hydroxy-2-methyl-pyrrolidine-1-carboxylate was consumed completely and a new spot was formed. The mixture was diluted with water and extracted with DCM. The combined organic layers were washed with brine, dried over Na₂SO₄ and the volatiles evaporated. The residue was purified by flash column chromatography (10-50% EtOAc in PE) to yield tert-butyl rac-(2R,4R)-2-methyl-4-methylsulfonyloxy-pyrrolidine-1-carboxylate (1410 mg, 5.05 mmol, 96.5% yield) as a yellow oil. ¹H NMR (400 MHz, CDCl₃) δ=5.21-5.15 (m, 1H), 4.06-3.71 (m, 2H), 3.56 (br d, J=10.4 Hz, 1H), 3.03 (s, 3H), 2.45 (br d, J=1.6 Hz, 1H), 1.90-1.81 (m, 1H), 1.47 (s, 9H), 1.30-1.26 (m, 3H).

### Step 2: tert-butyl (2S,4R)-4-cyano-2-methyl-pyrrolidine-1-carboxylate

[symbol]

To a mixture of tert-butyl (2S,4S)-2-methyl-4-methylsulfonyloxy-pyrrolidine-1-carboxylate (1.4 g, 5.0 mmol, 1.0 equiv.) in DMSO (15 mL) was added sodium cyanide (0.98 g, 20.0 mmol, 4.0 equiv.). The mixture was stirred at 80° C. for 16 hours. The mixture was poured into aq. sat. NaHCO₃ and extracted with EtOAc. The combined organic layers were washed with brine, dried over Na₂SO₄ and the volatiles evaporated. The residue was purified by column chromatography (10-50% EtOAc in PE) to yield tert-butyl (2S,4R)-4-cyano-2-methyl-pyrrolidine-1-carboxylate (805 mg, 3.83 mmol, 72.6% yield) as a colorless oil. LC-MS: m/z=155.1 [M−56+H]⁺ ESI pos. ¹H NMR (400 MHz, CDCl₃) δ=5.21-5.15 (m, 1H), 4.06-3.71 (m, 2H), 3.56 (br d, J=10.4 Hz, 1H), 3.03 (s, 3H), 2.45 (br d, J=1.7 Hz, 1H), 1.90-1.81 (m, 1H), 1.47 (s, 9H), 1.30-1.26 (m, 3H).

(NaCN work-up: Aqueous KOH (1M) was added to the combined aqueous phase to pH about 12. Then the mixture was poured into NaClO aqueous (5%, 1500 mL) and standing overnight and detected by analysis department recycled by special recycling bucket.)

### Step 3: (3R,5S)-5-methylpyrrolidine-3-carbonitrile; 2,2,2-trifluoroacetic acid

[symbol]

To a solution of tert-butyl (2S,4R)-4-cyano-2-methyl-pyrrolidine-1-carboxylate (1.5 g, 7.13 mmol, 1.0 equiv.) in DCM (10 mL) was added TFA (10.0 mL, 123.25 mmol, 17.3 equiv.). The mixture was stirred at 25° C. for 2 hours. The mixture was concentrated in vacuo to afford (3R,5S)-5-methylpyrrolidine-3-carbonitrile; 2,2,2-trifluoroacetic acid as light brown oil. The crude product was used into next step without further purification. ¹H NMR (400 MHz, DMSO-d₆) δ=3.63-3.43 (m, 4H), 2.60-2.52 (m, 1H), 1.86-1.76 (m, 1H), 1.32 (d, J=6.5 Hz, 3H).

### Step 4: (3R,5S)-1-(6-chloro-3-formyl-2-pyridyl)-5-methyl-pyrrolidine-3-carbonitrile

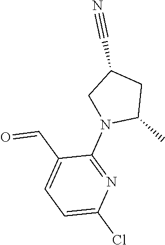 [SMILES: C[C@H]1C[C@@H](C#N)CN1c1nc(Cl)ccc1C=O]

A solution of (3R,5S)-5-methylpyrrolidine-3-carbonitrile; 2,2,2-trifluoroacetic acid (used as crude from the previous step) and DIPEA (6.18 mL, 37.37 mmol, 5.59 equiv.) in DMSO (20 mL) was stirred at RT for 5 minutes. Then, 6-chloro-2-fluoro-pyridine-3-carbaldehyde (CAS #1093880-37-5, 7.05 mmol, 1.05 equiv.) was added, and the reaction mixture stirred for 16 hours at RT. The reaction mixture was quenched with water and extracted with EtOAc. The combined organic layers were dried over Na₂SO₄ and concentrated under reduced pressure. The residue was purified by flash column chromatography (silica gel, 0-25% EtOAc in PE) to give (3R,5S)-1-(6-chloro-3-formyl-2-pyridyl)-5-methyl-pyrrolidine-3-carbonitrile.

### Step 5: mixture of (3R,5S)-1-[3-formyl-6-[6-[(6-methylpyridazin-3-yl)amino]benzimidazol-1-yl]-2-pyridyl]-5-methyl-pyrrolidine-3-carbonitrile and (3R,5S)-1-[3-formyl-6-[-5[(6-methylpyridazin-3-yl)amino]benzimidazol-1-yl]-2-pyridyl]-5-methyl-pyrrolidine-3-carbonitrile

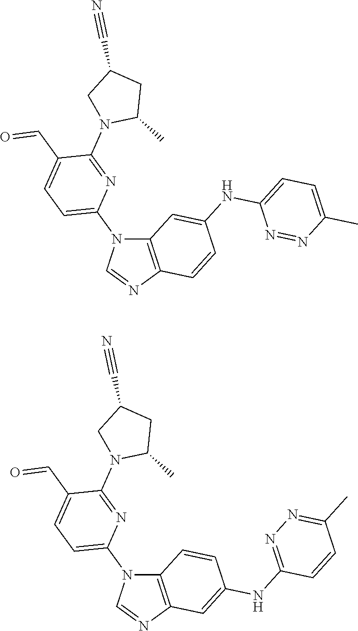 [SMILES: Cc1ccc(Nc2ccc3ncn(-c4ccc(C=O)c(N5C[C@H](C#N)C[C@@H]5C)n4)c3c2)nn1 ; Cc1ccc(Nc2ccc3c(c2)ncn3-c2ccc(C=O)c(N3C[C@H](C#N)C[C@@H]3C)n2)nn1]

A mixture of (3R,5S)-1-(6-chloro-3-formyl-2-pyridyl)-5-methyl-pyrrolidine-3-carbonitrile (1.0 equiv.), N-(6-methylpyridazin-3-yl)-1H-benzimidazol-5-amine (intermediate 1) (1.0 equiv.) and K₂CO₃ (2 equiv.) in DMSO (50 mL) was stirred at 50° C. for 12 hours. The mixture was cooled to RT and poured into H₂O (500 mL). A solid precipitated out. This was extracted with EtOAc (3×400 mL). The combined organic layers were concentrated. Purification by preparative HPLC (Phenomenex Luna C18 250 mm×50 mm×10 μm, gradient 5-40% CH₃CN in H₂O (with 0.1% TFA) over 20 min, then 100% CH₃CN (2 min), flow rate 100 mL/min) to yield (3R,5S)-1-[3-formyl-6-[6-[(6-methylpyridazin-3-yl)amino]benzimidazol-1-yl]-2-pyridyl]-5-methyl-pyrrolidine-3-carbonitrile and (3R,5S)-1-[3-formyl-6-[6-[(6-methylpyridazin-3-yl)amino]benzimidazol-1-yl]-2-pyridyl]-5-methyl-pyrrolidine-3-carbonitrile. LC-MS: m/z=439.2[M+H]⁺ ESI pos.

### Example 33

### (3R,5S)-1-[3-formyl-6-[5-[(6-methylpyridazin-3-yl)amino]benzimidazol-1-yl]-2-pyridyl]-5-methyl-pyrrolidine-3-carbonitrile

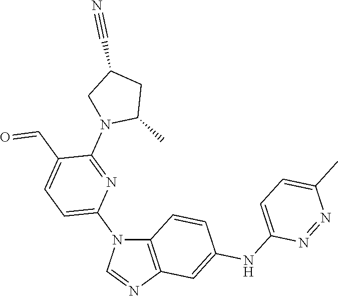 [SMILES: Cc1ccc(Nc2ccc3c(c2)ncn3-c2ccc(C=O)c(N3C[C@H](C#N)C[C@@H]3C)n2)nn1]

(3R,5S)-1-[3-formyl-6-[5-[(6-methylpyridazin-3-yl)amino]benzimidazol-1-yl]-2-pyridyl]-5-methyl-pyrrolidine-3-carbonitrile was synthesized according to the procedures described for Example 32. LC-MS: m/z=439.2[M+H]⁺ ESI pos.

### Example 34

### 5-methyl-1-[6-[5-[(6-methylpyridazin-3-yl)amino]benzimidazol-1-yl]-3-(2,2,2-trifluoroacetyl)-2-pyridyl]pyrazole-3-carbonitrile

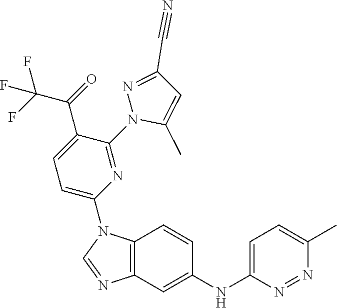 [SMILES: Cc1ccc(Nc2ccc3c(c2)ncn3-c2ccc(C(=O)C(F)(F)F)c(-n3nc(C#N)cc3C)n2)nn1]

### Step 1: 1-(6-chloro-2-fluoro-3-pyridyl)-2,2,2-trifluoro-ethanone

[symbol]

LDA (4.56 mL, 9.12 mmol, 1.2 equiv.) was added dropwise to a solution of 2-chloro-6-fluoropyridine (1.0 g, 7.6 mmol, 1.0 equiv.) in THF (20 mL) at −70° C. A yellow suspension was formed. The mixture was stirred at −70° C. for 1 hour, then N-methoxy-N-methyltrifluoroacetamide (1.26 g, 7.99 mmol, 1.05 equiv.) was added dropwise. After addition, the clear yellow solution was stirred at −70° C. for 1 hour. The mixture was quenched with 100 mL sat. aq. NH₄Cl, extracted with EtOAc and the organic layers were concentrated under reduced pressure. The residue was purified by flash column chromatography (silica gel, 25% EtOAc in PE) to yield 1-(6-chloro-2-fluoro-3-pyridyl)-2,2,2-trifluoro-ethanone (600 mg, 2.64 mmol, 34.7% yield) as light brown oil. LC-MS: m/z=246.1 [M+H₂O+H]⁺, ESI pos.

### Step 2: 1-[6-chloro-3-(2,2,2-trifluoroacetyl)-2-pyridyl]-5-methyl-pyrazole-3-carbonitrile

[symbol]

To mixture of 1-(6-chloro-2-fluoro-3-pyridyl)-2,2,2-trifluoro-ethanone (6.2 g, 27.25 mmol, 1.0 equiv.) and 5-methyl-1H-pyrazole-3-carbonitrile (2.91 g, 27.21 mmol, 1.0 equiv.) in DMSO (50 mL) was added DIPEA (7.9 mL, 54.5 mmol, 2.0 equiv.) dropwise at 0° C. After addition, the mixture was stirred at 20° C. for 3 hours. The mixture was quenched with 100 mL water, extracted with 100 mL EtOAc, and the organic layer concentrated under reduced pressure. The residue was purified by reversed phase preparative HPLC (Waters Xbridge BEH C18 150 mm×50 mm×10 μm, gradient 30-50% CH₃CN in H₂O (with 10 mM NH₄HCO₃) over 22 min, then 100% CH₃CN (5 min), flow rate 140 mL/min) to give 1-[6-chloro-3-(2,2,2-trifluoroacetyl)-2-pyridyl]-5-methyl-pyrazole-3-carbonitrile (3.2 g, 10.17 mmol, 37.3% yield) as a pink solid. LC-MS: m/z=315.1 [M+H]⁺, 333.1 [M+H₂O+H]⁺ ESI pos.

### Step 3: 5-methyl-1-[6-[5-[(6-methylpyridazin-3-yl)amino]benzimidazol-1-yl]-3-(2,2,2-trifluoroacetyl)-2-pyridyl]pyrazole-3-carbonitrile; formic acid and 5-methyl-1-[6-[6-[(6-methylpyridazin-3-yl)amino]benzimidazol-1-yl]-3-(2,2,2-trifluoroacetyl)-2-pyridyl]pyrazole-3-carbonitrile; formic acid

[symbol]

A mixture of 1-[6-chloro-3-(2,2,2-trifluoroacetyl)-2-pyridyl]-5-methyl-pyrazole-3-carbonitrile (0.4 g, 1.27 mmol, 1.0 equiv.), N-(6-methylpyridazin-3-yl)-1H-benzimidazol-5-amine (301.0 mg, 1.34 mmol, 1.05 equiv.) and DIPEA (0.45 mL, 2.54 mmol, 2.0 equiv.) in DMF (10 mL) was stirred at 100° C. for 16 hours. The mixture was purified by preparative HPLC (Shim-pack C18 150 mm×25 mm×10 μm, gradient 1-30% CH₃CN in H₂O (with 0.225% formic acid) over 10 min, then 100% CH₃CN (2 min), flow rate 25 mL/min, 1 injection) to yield 5-methyl-1-[6-[5-[(6-methylpyridazin-3-yl)amino]benzimidazol-1-yl]-3-(2,2,2-trifluoroacetyl)-2-pyridyl]pyrazole-3-carbonitrile; formic acid (170 mg, 26.6% yield) as dark brown solid. LC-MS: m/z=504.1 [M+H]⁺, ESI pos. and 5-methyl-1-[6-[6-[(6-methylpyridazin-3-yl)amino]benzimidazol-1-yl]-3-(2,2,2-trifluoroacetyl)-2-pyridyl]pyrazole-3-carbonitrile; formic acid (120 mg, 18.8% yield) as dark brown solid. LC-MS: m/z=504.1 [M+H]⁺, ESI pos.

### Example 35

### 1-[3-acetyl-6-[6-keto-7,7-dimethyl-5-(6-methylpyridazin-3-yl)pyrrolo[2,3-f]benzimidazol-1-yl]-2-pyridyl]-5-methyl-pyrazole-3-carbonitrile

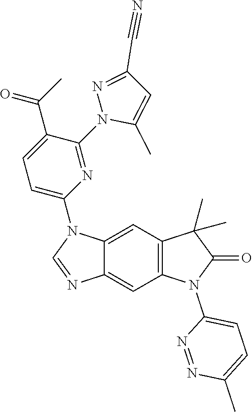 [SMILES: CC(=O)c1ccc(-n2cnc3cc4c(cc32)C(C)(C)C(=O)N4c2ccc(C)nn2)nc1-n1nc(C#N)cc1C]

1-[3-acetyl-6-[6-keto-7,7-dimethyl-5-(6-methylpyridazin-3-yl)pyrrolo[2,3-f]benzimidazol-1-yl]-2-pyridyl]-5-methyl-pyrazole-3-carbonitrile was made according to methods described herein and methods known to those skilled in the art. LC-MS: m/z=518.2 [M+H]⁺, ESI pos.

### Example 36

### 1-[3-formyl-6-[5-[(6-methylpyridazin-3-yl)amino]benzimidazol-1-yl]-2-pyridyl]-5-methyl-pyrazole-3-carbonitrile

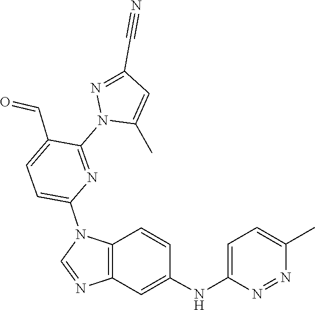 [SMILES: Cc1ccc(Nc2ccc3c(c2)ncn3-c2ccc(C=O)c(-n3nc(C#N)cc3C)n2)nn1]

1-[3-formyl-6-[5-[(6-methylpyridazin-3-yl)amino]benzimidazol-1-yl]-2-pyridyl]-5-methyl-pyrazole-3-carbonitrile was synthesized analogous to Example 27 using 1-(6-chloro-3-formyl-2-pyridyl)-5-methyl-pyrazole-3-carbonitrile. LC-MS: m/z=436.3 [M+H]⁺, ESI pos.

### Example 37

### 1-[3-acetyl-6-[5-[(2-keto-1-methyl-3-pyridyl)amino]benzimidazol-1-yl]-2-pyridyl]-5-methyl-pyrazole-3-carbonitrile

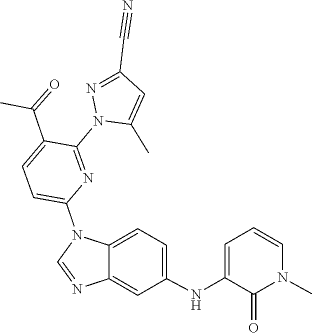 [SMILES: CC(=O)c1ccc(-n2cnc3cc(Nc4cccn(C)c4=O)ccc32)nc1-n1nc(C#N)cc1C]

1-[3-acetyl-6-[5-[(2-keto-1-methyl-3-pyridyl)amino]benzimidazol-1-yl]-2-pyridyl]-5-methyl-pyrazole-3-carbonitrile was synthesized according to the methods described herein, using suitable intermediates 18, 19 and 3-chloro-1-methyl-pyridin-2-one (CAS: 123062-64-6). LC-MS: m/z=465.4 [M+H]⁺, ESI pos.

### Example 38

### 1-[3-acetyl-6-[5-(3-methoxy-1-methyl-pyrazol-4-yl)benzimidazol-1-yl]-2-pyridyl]-5-methyl-pyrazole-3-carbonitrile

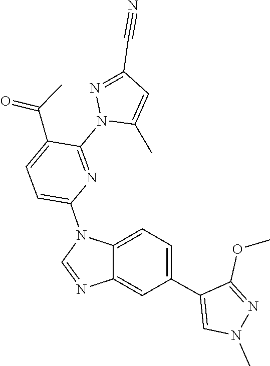 [SMILES: COc1nn(C)cc1-c1ccc2c(c1)ncn2-c1ccc(C(C)=O)c(-n2nc(C#N)cc2C)n1]

1-[3-acetyl-6-[5-(3-methoxy-1-methyl-pyrazol-4-yl)benzimidazol-1-yl]-2-pyridyl]-5-methyl-pyrazole-3-carbonitrile was synthesized according to the methods described herein and methods known to those skilled in the art. LC-MS: m/z=453.4 [M+H]⁺, ESI pos.

### Example 39

### 1-[3-acetyl-6-[5-[(6-pyrrolidin-2-ylpyridazin-3-yl)amino]benzimidazol-1-yl]-2-pyridyl]-5-methyl-pyrazole-3-carbonitrile

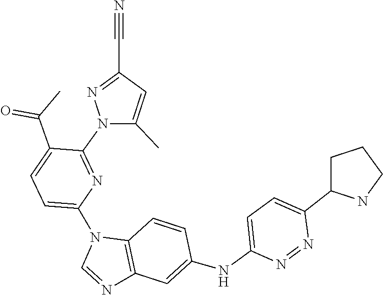

### Step 1: tert-butyl 2-(6-aminopyridazin-3-yl)pyrrolidine-1-carboxylate

An oven-dried 15 mL vial equipped with magnetic stir bar was charged with 6-iodopyridazin-3-amine (1.00 g, 4.52 mmol, 1.0 eq), 1-(tert-butoxycarbonyl)pyrrolidine-2-carboxylic acid (1.27 g, 5.88 mmol, 1.3 eq), Ir[dF(CF₃)ppy]2(dtbpy)(PF₆) (51 mg, 0.05 mmol, 0.01 eq), NiCl₂.dtbbpy (90 mg, 0.23 mmol, 0.05 eq), Cs₂CO₃ (2.21 g, 6.79 mmol, 1.5 eq) in DMA (40 mL). The reaction mixture was bubbled with N₂ for 10 minutes then irradiated with two 34 W blue LED lamps (approximately 7 cm away from the light source to keep the reaction temperature at 25° C.) for 12 hours. The reaction mixture was poured into H₂O (150 mL) and extracted with EtOAc (3×40 mL). The combined organic layers were dried over Na₂SO₄, filtered and concentracted. The residue was purified by preparative HPLC (Waters Xbridge 150 mm×25 mm×5 μm, water (10 mM NH₄HCO₃)-ACN) to give tert-butyl 2-(6-aminopyridazin-3-yl)pyrrolidine-1-carboxylate (120 mg, 0.45 mmol, 10% yield) as white solid. LC-MS: m/z=265.1 [M+H]⁺, ESI pos.

### Step 2: tert-butyl 2-[6-[[1-[5-acetyl-6-(3-cyano-5-methyl-pyrazol-1-yl)-2-pyridyl]benzimidazol-5-yl]amino]pyridazin-3-yl]pyrrolidine-1-carboxylate

To a solution of tert-butyl 2-(6-aminopyridazin-3-yl)pyrrolidine-1-carboxylate (69 mg, 0.26 mmol, 1.1 eq) and 1-[3-acetyl-6-(5-bromobenzimidazol-1-yl)-2-pyridyl]-5-methyl-pyrazole-3-carbonitrile (100 mg, 0.24 mmol, 1.0 eq, prepared in example 65, step 1) in 1,4-dioxane (8 mL) was added Cs₂CO₃ (232 mg, 0.71 mmol, 3.0 eq). The mixture was bubbled with N₂ for 10 minutes and [tBuBrettPhos Pd(allyl)]OTf (19 mg, 0.02 mmol, 0.1 eq) was added. The reaction mixture was stirred at 80° C. for 2 hours. The mixture was cooled to RT, poured into H₂O (20 mL) and extracted with EtOAc (3×20 ml). The combined organic layers were washed with brine (20 mL), dried over Na₂SO₄, filtered and concentrated. The residue was purified by preparative TLC (DCM: MeOH 10:1) to give tert-butyl 2-[6-[[1-[5-acetyl-6-(3-cyano-5-methyl-pyrazol-1-yl)-2-pyridyl]benzimidazol-5-yl]amino]pyridazin-3-yl]pyrrolidine-1-carboxylate (110 mg, 0.18 mmol, 77% yield) as light brown solid. LC-MS: m/z=605.2 [M+H]⁺, ESI pos.

### Step 3: 1-[3-acetyl-6-[5-[(6-pyrrolidin-2-ylpyridazin-3-yl)amino]benzimidazol-1-yl]-2-pyridyl]-5-methyl-pyrazole-3-carbonitrile

To a solution of tert-butyl 2-[6-[[1-[5-acetyl-6-(3-cyano-5-methyl-pyrazol-1-yl)-2-pyridyl]benzimidazol-5-yl]amino]pyridazin-3-yl]pyrrolidine-1-carboxylate (30 mg, 0.05 mmol, 1.0 eq) in DCM (1.5 mL) was added HCl (4 M in dioxane) (0.8 mL). The mixture was stirred at RT for 1 hour. The reaction mixture was concentrated. The residue was purified by preparative HPLC (Phenomenex Synergi C18 150 mm×25 mm×10 μm, water with FA-ACN) to give 1-[3-acetyl-6-[5-[(6-pyrrolidin-2-ylpyridazin-3-yl)amino]benzimidazol-1-yl]-2-pyridyl]-5-methyl-pyrazole-3-carbonitrile (20 mg, 0.04 mmol, 78% yield) as an off white solid. LC-MS: m/z=505.2 [M+H]⁺, ESI pos.

### Example 40

### 1-[3-formyl-6-[6-[(6-methylpyridazin-3-yl)amino]benzimidazol-1-yl]-2-pyridyl]-5-methyl-pyrazole-3-carbonitrile

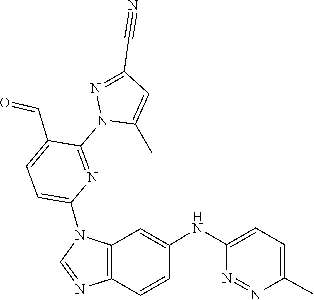 [SMILES: Cc1ccc(Nc2ccc3ncn(-c4ccc(C=O)c(-n5nc(C#N)cc5C)n4)c3c2)nn1]

1-[3-formyl-6-[5-[(6-methylpyridazin-3-yl)amino]benzimidazol-1-yl]-2-pyridyl]-5-methyl-pyrazole-3-carbonitrile was synthesized analogous to Example 27 using 1-(6-chloro-3-formyl-2-pyridyl)-5-methyl-pyrazole-3-carbonitrile. LC-MS: m/z=436.3 [M+H]⁺, ESI pos.

### Enzymatic Activity Assay:

### Assay Description SIK1-3, RapidFire:

In the presence of SIK2 (resp. SIK1 or SIK3) and ATP the CHK-peptide (KKKVSRSGLYRSPSMPENLNRPR with C-terminal arginine amide modification) were phosphorylated at one of the four feasible serine's. Only one phosphorylation is observed under the assay conditions. 60 nl of each compound dilution series (12 point; dilution factor 3, generally 30 μM to 170 pM) in DMSO were transferred by acoustic dispensing to the assay plate and 30 min pre-incubated (ambient temperature) after the addition of 5 μl SIK1 (5 nM) resp. 5 μl SIK2 (0.5 nM) or 7 μl SIK3 (1.5 nM) in assay-buffer (12.5 mM HEPES (pH 7.0), 10 mM magnesium acetate, 0.005% BSA). 10 μM CHK-peptide solution and 5 μl 100 μM ATP for SIK1 & SIK2 resp. 3 μl for SIK3 in assay-buffer were added and incubated ambient for 45 min. 40 μl 0.125% formic acid in water were added to quench the reaction. RapidFire (RF) Mass Spectrometry was utilized for data generation as described below. The multiple charged species (3-5 charges) for the phosphorylated and non-phosphorylated form measured by MRM (Multiple Reaction Monitoring; API5000 or 6500+) or EIC (Extracted Ion Current; QToF) were summed up and the ratio calculated (sum phosphorylated species/sum all species) for data evaluation. Normalization was performed by Genedata software based on the non-inhibition control DMSO and the commercially available SIK inhibitor @ 1 μM YKL-05-099 (CAS number 1936529-65-5). The results of the assay are expressed in half-maximal inhibitory concentrations (IC50s) and are summarized below in Table 1.

### RapidFire Setup:

Samples were aspirated by vacuum for max. 600 ms and loaded to C4-cartridge (Agilent; #G9203A) for 3000 ms@1.5 ml/min with 0.1% formic acid in water. Afterwards samples were transferred to the API5000 (API6500+) or QToF mass spectrometer for 4000 ms@1.25 ml/min with 90% acetonitrile; 10% water; 0.007% TFA; 0.093 formic acid. The cartridge was reconditioned for additional 500 ms with 0.1% formic acid in water

### MS-Setup Sciex API5000/API6500+:

All MS analyses using the following MS-setup in MRM mode: Electrospray positive; Ion Spray Voltage: 4000V; Temperature: 550° C.; Collision Gas: 5; Curtain Gas: 15; Gas1: 40; Gas 2: 42; EP: 10

| Name | Q1 | Q3 | Time(ms) | DP | CE | CXP |
|---|---|---|---|---|---|---|
| CHK(4+) | 676.0 | 84.2 | 50 | 46 | 99 | 18 |
| CHK(5+) | 541.0 | 84.2 | 50 | 51 | 71 | 36 |
| pCHK(4+) | 696.0 | 84.2 | 50 | 66 | 105 | 18 |
| pCHK(5+) | 557.0 | 84.2 | 50 | 51 | 53 | 4 |

### MS-Setup Agilent QToF 6545

All MS analyses using the following MS-setup in Mode MS: Dual AJS Electrospray positive; VCap: 3000V; Drying & Sheath gas: 340° C.@81/min; Nebulizer: 60 psig; Nozzle Voltage: 2000V; Fragmentor: 130V; Skimmer: 35V; Oct1 RF Vpp: 700V; Ref masses on@5spectra/s

| Name | EIC | Width |
|---|---|---|
| CHK(3+) | 900.8345 | 50 ppm |
| CHK(4+) | 675.8789 | 50 ppm |
| CHK(5+) | 540.9048 | 50 ppm |
| pCHK(3+) | 927.4961 | 50 ppm |
| pCHK(4+) | 695.8747 | 50 ppm |

TABLE 1
IC50 values for inhibition of SIK1, SIK2, and SIK3:

| | SIK1 RF | SIK2 RF | SIK3 RF |
|---|---|---|---|
| Example | IC50 (μM) | IC50 (μM) | IC50 (μM) |
| 1 | 0.0151 | 0.0142 | 0.0572 |
| 2 | 0.0140 | 0.0038 | 0.0972 |
| 3 | 0.0155 | 0.0078 | 0.1559 |
| 4 | 0.0034 | 0.0033 | 0.1145 |
| 5 | 0.0074 | 0.0095 | 0.3308 |
| 6 | 0.0028 | 0.0025 | 0.0399 |
| 7 | 0.0297 | 0.0182 | 0.1879 |
| 8 | 0.0274 | 0.0162 | 0.2602 |
| 9 | 0.0062 | 0.0065 | 0.1462 |
| 10 | 0.0017 | 0.0012 | 0.0449 |
| 11 | 0.0063 | 0.0158 | 0.2063 |
| 12 | 0.0017 | 0.0022 | 0.0692 |
| 13 | 22.5089 | 0.9948 | >30.0000 |
| 14 | 7.4809 | 5.1452 | 17.9962 |
| 15 | 1.5668 | 0.8277 | 4.0043 |
| 16 | 0.2603 | 0.2415 | 1.6671 |
| 17 | 14.9353 | 5.0682 | 21.5583 |
| 18 | 9.4682 | 5.7677 | 13.5291 |
| 19 | 1.7722 | 5.7410 | >10.0000 |
| 20 | 12.1196 | 6.2446 | >30.0000 |
| 21 | 0.6590 | 0.8779 | 3.4952 |
| 22 | 19.8428 | 7.4097 | 12.2304 |
| 23 | 0.3612 | 0.0152 | 0.0049 |
| 24 | 0.6053 | 0.2056 | 0.0957 |
| 25 | 0.9137 | 0.1709 | 0.2570 |
| 26 | 17.8175 | 0.1535 | 0.0494 |
| 27 | 0.6688 | 0.0066 | 0.0081 |
| 28 | 0.3085 | 0.1111 | 0.1843 |
| 29 | 1.461 | 0.0867 | |
| 30 | 0.547 | 0.8159 | |
| 31 | 0.013 | 0.0091 | 1.2259 |
| 32 | 0.043 | 0.0199 | 0.9879 |
| 33 | 0.076 | 0.0951 | 0.5622 |
| 34 | 2.331 | 0.0414 | 0.0317 |
| 35 | 0.101 | 0.1149 | 0.1500 |
| 36 | 0.274 | 0.0052 | 0.0007 |
| 37 | >10.000 | 0.5021 | 0.2080 |
| 38 | 3.485 | 0.7541 | 1.7922 |
| 39 | 6.581 | 0.0437 | 0.7279 |
| 40 | >30.000 | 0.5359 | 8.7959 |
| 41 | 0.099 | 0.0036 | 0.0012 |
| 42 | 0.919 | 0.4727 | 7.6466 |
| 43 | 4.713 | 0.7876 | 1.0036 |
| 44 | 0.065 | 0.0387 | 0.0925 |
| 45 | 0.004 | 0.0048 | 0.0531 |
| 46 | 1.457 | 0.0149 | 0.0271 |
| 47 | 0.039 | 0.0720 | 0.2259 |
| 48 | 5.305 | 0.2482 | 0.2675 |
| 49 | >30.000 | 3.2894 | 0.3740 |
| 50 | 15.120 | 0.2508 | 0.1130 |

### Specific Numbered Embodiments

1. A compound of formula (I)

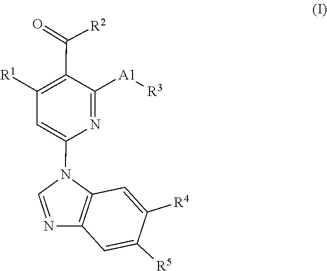

wherein
R¹ is hydrogen or alkoxy;
R² is hydrogen, alkyl, amino, alkylamino, dialkylamino, haloalkyl, haloalkylamino cycloalkylamino, hydroxy, alkoxy, cycloalkyl, cycloalkyloxy or haloalkoxy;
A1 is —O—, —NR⁶— or a bond;
R⁶ is hydrogen or alkyl;
R³ is alkyl, haloalkyl, hydroxyalkyl, heterocycloalkyl, heteroaryl, phenyl, heteroarylalkyl, phenylalkyl, cycloalkyl, cycloalkylalkyl, (amino)(phenyl)alkyl, (amino)(halophenyl)alkyl or (amino)(heteroaryl)alkyl, wherein heterocycloalkyl, heteroaryl, phenyl, heteroarylalkyl, phenylalkyl, cycloalkyl and cycloalkylalkyl are optionally substituted with 1, 2 or 3 substituents independently selected from R⁷;
each R⁷ is independently selected from alkoxy, alkylamino, alkyl, aminocarbonyl, amino, cyano, cycloalkylamino, haloalkyl, halocycloalkyl, halogen, heteroaryl, hydroxycarbonylamino, alkoxyalkyl, alkylaminocarbonyl, alkylsulfonyl, aminocarbonyl, hydroxy, cycloalkylalkyl, haloalkoxy, heterocycloalkyl and cycloalkyl;
R⁴ is hydrogen, alkyl, halogen, cyano, haloalkyl, alkoxy, alkoxyalkyl, dialkylaminoalkyl, dialkylamino, alkylamino, alkylaminoalkyl, cycloalkyl, cycloalkylamino, cycloalkyloxy, cycloalkylalkyl, heteroarylalkyl, heteroarylamino, heteroaryloxy, heterocycloalkyl, heterocycloalkylamino, heterocycloalkyloxy or heterocycloalkylalkyl; wherein cycloalkyl, cycloalkylamino, cycloalkyloxy, cycloalkylalkyl, heteroarylalkyl, heteroarylamino, heteroaryloxy, heterocycloalkyl, heterocycloalkylamino, heterocycloalkyloxy and heterocycloalkylalkyl are optionally substituted with 1, 2 or 3 substituents independently selected from R⁸;
each R⁸ is independently selected from alkyl, halogen, cyano, alkylsulfonyl, alkylaminocarbonyl, heterocycloalkyl and alkoxyheterocycloalkylalkyl;
R⁵ is hydrogen, alkyl, halogen, cyano, haloalkyl, alkoxy, alkoxyalkyl, dialkylaminoalkyl, dialkylamino, alkylamino, alkylaminoalkyl, alkylsulfonyl, cycloalkyl, cycloalkylamino, cycloalkyloxy, cycloalkylalkyl, heteroarylalkyl, heteroarylamino, heteroaryloxy, heterocycloalkyl, heterocycloalkylamino, heterocycloalkyloxy or heterocycloalkylalkyl; wherein cycloalkyl, cycloalkylamino, cycloalkyloxy, cycloalkylalkyl, heteroarylalkyl, heteroarylamino, heteroaryloxy, heterocycloalkyl, heterocycloalkylamino, heterocycloalkyloxy and heterocycloalkylalkyl are optionally substituted with 1, 2 or 3 substituents independently selected from R⁹;
or R⁴ and R⁵ together with the carbon they are attached to form a 5 to 7 membered heterocyclic ring optionally substituted with one, two or three substituents independently selected from alkyl, cyano, halogen, haloalkyl, alkoxy, heteroaryl and alkylheteroaryl;
each R⁹ is independently selected from alkoxy, halogen, dialkylaminocarbonyl, alkyl, alkoxyalkoxy, alkoxyheterocycloalkylalkyl, alkoxyheterocycloalkylcarbonyl, haloalkyl, haloalkoxy, heterocycloalkylalkoxy, heterocycloalkyl, heterocycloalkyloxy, hydroxy, alkylheterocycloalkyl, alkylheterocycloalkylalkyl, heterocycloalkylalkyl, alkylsulfonyl, (alkyl)heterocycloalkyl, alkylheterocycloalkyloxy, heterocycloalkylheterocycloalkyl, (heterocycloalkyl)heterocycloalkyl, CH₃—O—(CH₂—CH₂—O)₇—, alkylaminocarbonyl and cyano;
or a pharmaceutically acceptable salt thereof.
2. A compound according to embodiment 1, wherein R¹ is hydrogen or methoxy.
3. A compound according to embodiment 1 or 2, wherein R¹ is hydrogen.
4. A compound according to any one of embodiments 1 to 3, wherein R¹ is alkoxy, in particular methoxy.
5. A compound according to any one of embodiments 1 to 4, wherein R² is hydrogen, methyl, amino, methylamino, ethylamino, dimethylamino, trifluoromethyl, trifluoromethylamino, cyclopropylamino, hydroxy or methoxy.
6. A compound according to any one of embodiments 1 to 5, wherein R² is methyl, amino, methylamino, ethylamino, trifluoromethylamino, cyclopropylamino, hydroxy or methoxy.
7. A compound according to any one of embodiments 1 to 5, wherein R² is hydrogen, dimethylamino or trifluoromethyl.
8. A compound according to any one of embodiments 1 to 5, wherein R² is amino or alkyl.
9. A compound according to any one of embodiments 1 to 5, wherein R² is amino or methyl.
10. A compound according to any one of embodiments 1 to 9, wherein A1 is —O— or a bond.
11. A compound according to any one of embodiments 1 to 9, wherein A1 is —O—.
12. A compound according to any one of embodiments 1 to 9, wherein A1 is a bond.
13. A compound according to any one of embodiments 1 to 9, wherein A1 is —NR⁶—.
14. A compound according to any one of embodiments 1 to 13, wherein R⁶ is hydrogen or methyl.
15. A compound according to any one of embodiments 1 to 14, wherein R⁵ is hydrogen.
16. A compound according to any one of embodiments 1 to 14, wherein R⁶ is alkyl.
17. A compound according to any one of embodiments 1 to 16, wherein R⁶ is methyl.
18. A compound according to any one of embodiments 1 to 17, wherein R³ is alkyl, haloalkyl, heterocycloalkyl, heteroaryl, phenyl, phenylalkyl, (amino)(phenyl)alkyl, (amino)(halophenyl)alkyl or (amino)(heteroaryl)alkyl, wherein heterocycloalkyl, heteroaryl, phenyl, heteroarylalkyl and phenylalkyl are optionally substituted with 1, 2 or 3 substituents independently selected from R⁷; and wherein each R⁷ is independently selected from halogen, alkyl, cyano, alkoxy and haloalkyl.
19. A compound according to any one of embodiments 1 to 18, wherein R³ is methyl difluoropropyl, phenyl, phenylmethyl, phenylethyl, 2-amino-1-(3-chlorophenyl)ethyl, 3-amino-1-phenyl-propyl, 3-amino-1-(3-thienyl)propyl, 2-thienylmethyl, heterocycloalkyl or heteroaryl, wherein heterocycloalkyl, heteroaryl, phenyl, phenylmethyl and phenylethyl are optionally substituted with 1, 2 or 3 substituents independently selected from R⁷; and
wherein each R⁷ is independently selected from chloro, methyl, cyano, methoxy and difluoromethyl.
20. A compound according to any one of embodiments 1 to 19, wherein the heterocycloalkyl of substituent R³ is selected from 2-morpholino, pyrrolidinyl, piperidyl, 2-oxopyrrolidinyl, (1,1-dioxo-1,2-thiazolidinyl), (4,5,6,7-tetrahydropyrazolo[4,3-c]pyridinyl), pyrrolidinyl, [rac-(3aR,6aS)-2,3,3a,5,6,6a-hexahydro-1H-pyrrolo[3,2-b]pyrrolyl], [rac-(3aS,6aR)-2,3,3a,5,6,6a-hexahydro-1H-pyrrolo[3,2-b]pyrrolyl], [3-oxo-piperazinyl], (4-oxo-6,7-dihydro-5H-pyrazolo[1,5-a]pyrazinyl), (6,7-dihydro-4H-pyrazolo[4,3-c]pyridinyl), azetidinyl, pyrrolidinyl, (3-oxo-1,5,6,8-tetrahydrooxazolo[3,4-a]pyrazinyl), piperazinyl, 4,7-diazaspiro[2.5]octanyl, (2-oxa-5,8-diazaspiro[3.5]nonanyl), 3-azabicyclo[3.2.0]heptanyl), (5-azaspiro[2.4]heptanyl), (2-azabicyclo[2.2.1]heptanyl), morpholinyl, 4-oxa-7-azaspiro[2.5]octanyl, (3-azabicyclo[3.1.0]hexanyl), (6,7-dihydro-4H-pyrazolo[4,3-c]pyridinyl), 2-oxa-7-azaspiro[3.4]octanyl and [(1S,5R,7R)-4-oxo-3-oxa-9-azatricyclo[5.3.0.01,5]decan-9-yl]; and
wherein the heteroaryl of substituent R³ is selected from 2-oxo-pyridyl, pyrazolyl, pyridyl, pyridazinyl, isoxazol-4-yl, pyrimidinyl, 1H-benzotriazolyl, furyl, [6-oxo-1H-pyridazinyl] and triazolyl.
21. A compound according to any one of embodiments 1 to 20, wherein the heterocycloalkyl of substituent R³ is selected from 2-oxo-1-piperidyl, 2-oxopyrrolidin-1-yl, 1-piperidyl, pyrrolidin-1-yl and [(1S,5R,7R)-4-oxo-3-oxa-9-azatricyclo[5.3.0.01,5]decan-9-yl]; and
wherein the heteroaryl substituent of R³ is pyrazol-1-yl.
22. A compound according to any one of embodiments 1 to 21, wherein
R⁴ is hydrogen, halogen, cyano, haloalkyl, alkoxy, alkoxyalkyl, dialkylaminoalkyl, dialkylamino, alkylamino, alkylaminoalkyl, cycloalkyl, cycloalkylamino, cycloalkyloxy, cycloalkylalkyl, heteroarylalkyl, heteroarylamino, heteroaryloxy, heterocycloalkyl, heterocycloalkylamino, heterocycloalkyloxy or heterocycloalkylalkyl; wherein cycloalkyl, cycloalkylamino, cycloalkyloxy, cycloalkylalkyl, heteroarylalkyl, heteroarylamino, heteroaryloxy, heterocycloalkyl, heterocycloalkylamino, heterocycloalkyloxy and heterocycloalkylalkyl are optionally substituted with 1, 2 or 3 substituents independently selected from R⁸;
each R⁸ is independently selected from alkyl, halogen, cyano, alkylsulfonyl, alkylaminocarbonyl, heterocycloalkyl and alkoxyheterocycloalkylalkyl;
R⁵ is hydrogen, halogen, cyano, haloalkyl, alkoxy, alkoxyalkyl, dialkylaminoalkyl, dialkylamino, alkylamino, alkylaminoalkyl, alkylsulfonyl, cycloalkyl, cycloalkylamino, cycloalkyloxy, cycloalkylalkyl, heteroarylalkyl, heteroarylamino, heteroaryloxy, heterocycloalkyl, heterocycloalkylamino, heterocycloalkyloxy or heterocycloalkylalkyl; wherein cycloalkyl, cycloalkylamino, cycloalkyloxy, cycloalkylalkyl, heteroarylalkyl, heteroarylamino, heteroaryloxy, heterocycloalkyl, heterocycloalkylamino, heterocycloalkyloxy and heterocycloalkylalkyl are optionally substituted with 1, 2 or 3 substituents independently selected from R⁹;
or R⁴ and R⁵ together with the carbon they are attached to form a 5 to 7 membered heterocyclic ring optionally substituted with one, two or three substituents independently selected from alkyl, cyano, halogen, haloalkyl, alkoxy, heteroaryl and alkylheteroaryl;
each R⁹ is independently selected from alkoxy, halogen, dialkylaminocarbonyl, alkyl, alkoxyalkoxy, alkoxyheterocycloalkylalkyl, alkoxyheterocycloalkylcarbonyl, haloalkyl, haloalkoxy, heterocycloalkylalkoxy, heterocycloalkyl, heterocycloalkyloxy, hydroxy, alkylheterocycloalkyl, alkylheterocycloalkylalkyl, heterocycloalkylalkyl, alkylsulfonyl, (alkyl)heterocycloalkyl, alkylheterocycloalkyloxy, heterocycloalkylheterocycloalkyl, (heterocycloalkyl)heterocycloalkyl, CH₃—O—(CH₂—CH₂—O)₇—, alkylaminocarbonyl and cyano;
with the proviso that only one of R⁴ and R⁵ can be hydrogen.
23. A compound according to any one of embodiments 1 to 22, wherein
R⁴ is hydrogen, fluoro, cyano, trifluoromethyl, methoxy, methoxyethyl, dimethylaminoethyl, cyclopropylcarbonyl, morpholinoethyl, (1,1-dioxo-1,2-thiazolidin-2-yl)methyl, (2-oxopyrrolidin-1-yl)methyl, (2-oxo-1-piperidyl)methyl, heteroaryl or heterocycloalkyl, wherein heteroaryl and heterocycloalkyl are optionally substituted with 1, 2 or 3 substituents independently selected from R⁸; each R⁸ is independently selected from methyl, fluoro, cyano, methylsulfonyl, oxetan-3-yl and (3-methoxyazetidin-1-yl)methyl;
R⁵ is hydrogen, alkoxy, heterocycloalkylalkoxy, heterocycloalkylamino, heteroarylamino, heteroarylalkyl or heteroaryl, wherein heterocycloalkylalkoxy, heterocycloalkylamino, heteroarylamino, heteroarylalkyl and heteroaryl are optionally substituted with 1, 2 or 3 substituents independently selected from R⁹;
or R⁴ and R⁵ together with the carbon they are attached to form a 5 to 7 membered heterocyclic ring optionally substituted with one, two or three substituents independently selected from alkyl and alkylheteroaryl;
each R⁹ is independently selected from alkyl, alkoxy, halogen, haloalkyl, dialkylaminocarbonyl, heterocycloalkyl and (heterocycloalkyl)heterocycloalkyl;
with the proviso that only one of R⁴ and R⁵ can be hydrogen.
24. A compound according to any one of embodiments 1 to 23, wherein R⁴ is hydrogen, fluoro, cyano, trifluoromethyl, methoxy, methoxyethyl, dimethylaminoethyl, cyclopropylcarbonyl, morpholinoethyl, (1,1-dioxo-1,2-thiazolidin-2-yl)methyl, (2-oxopyrrolidin-1-yl)methyl, (2-oxo-1-piperidyl)methyl, heteroaryl or heterocycloalkyl, wherein heteroaryl and heterocycloalkyl are optionally substituted with 1, 2 or 3 substituents independently selected from R⁸; and
each R⁸ is independently selected from methyl, fluoro, cyano, methylsulfonyl, oxetan-3-yl and (3-methoxyazetidin-1-yl)methyl.
25. A compound according to any one of embodiments 1 to 24, wherein R⁴ is hydrogen, fluoro, alkoxy, heteroarylamino or heteroarylalkyl, wherein heteroarylamino and heteroarylalkyl are optionally substituted with 1, 2 or 3 substituents independently selected from R⁸; and
each R⁸ is independently selected from alkyl.
26. A compound according to any one of embodiments 1 to 25, wherein R⁴ is hydrogen, fluoro, alkoxy, (pyridazin-3-yl)amino or (pyridazin-3-yl)alkyl wherein (pyridazin-3-yl)amino or (pyridazin-3-yl)alkyl are optionally substituted with 1, 2 or 3 substituents independently selected from R⁸; and
each R⁸ is independently selected from alkyl.
27. A compound according to any one of embodiments 1 to 26, wherein R⁴ is hydrogen, fluoro, methoxy, (pyridazin-3-yl)amino or (pyridazin-3-yl)alkyl, wherein (pyridazin-3-yl)amino and (pyridazin-3-yl)alkyl are optionally substituted with methyl.
28. A compound according to any one of embodiments 1 to 27, wherein the heterocycloalkyl of substituent R⁴ is selected from piperidyl, piperazinyl, pyrrolidinyl, 2,3-dihydropyridazino[4,5-b][1,4]oxazinyl, pyrrolidinyl, 2-oxo-pyrimidinyl, 2-oxa-5-azaspiro[3.4]octanyl and oxetanyl; and
wherein the heteroaryl of substituent R⁴ is selected from pyridazinyl and pyridyl.
29. A compound according to any one of embodiments 1 to 28, wherein the heterocycloalkyl of substituent R⁴ is selected from 4-piperidyl, piperazin-1-yl, pyrrolidin-3-yl, 2,3-dihydropyridazino[4,5-b][1,4]oxazin-8-yl, pyrrolidin-1-yl, 2-oxo-pyrimidin-4-yl, 2-oxa-5-azaspiro[3.4]octan-5-yl and oxetan-3-yl; and
wherein the heteroaryl of substituent R⁴ is selected from pyridazin-3-yl and 3-pyridyl.
30. A compound according to any one of embodiments 1 to 29, wherein R⁴ is hydrogen.
31. A compound according to any one of embodiments 1 to 29, wherein R⁴ is alkoxy.
32. A compound according to any one of embodiments 1 to 29, wherein R⁴ is methoxy.
33. A compound according to any one of embodiments 1 to 32, wherein R⁵ is hydrogen, alkoxy, heterocycloalkylalkoxy, heterocycloalkylamino, heteroarylamino, heteroarylalkyl or heteroaryl, wherein heterocycloalkylalkoxy, heterocycloalkylamino, heteroarylamino, heteroarylalkyl and heteroaryl are optionally substituted with 1, 2 or 3 substituents independently selected from R⁹; and
wherein each R⁹ is independently selected from alkyl, alkoxy, halogen, haloalkyl, dialkylaminocarbonyl, heterocycloalkyl and (heterocycloalkyl)heterocycloalkyl.
34. A compound according to any one of embodiments 1 to 32, wherein R⁵ is hydrogen, methoxy, 2-morpholinoethoxy, (pyridazin-3-yl)amino, (pyridazin-3-yl)alkyl, pyrazol-4-yl, (2-oxo-3-pyridyl)amino, wherein (pyridazin-3-yl)amino, (pyridazin-3-yl)alkyl, pyrazol-4-yl and (2-oxo-3-pyridyl)amino are optionally substituted with 1, 2 or 3 substituents independently selected from R⁹; and
wherein each R⁹ is independently selected from methyl, fluoro, halogen, dimethylaminocarbonyl, heterocycloalkyl and (heterocycloalkyl)heterocycloalkyl.
35. A compound according to any one of embodiments 1 to 34, wherein R⁵ is (pyridazin-3-yl)amino optionally substituted with alkyl.
36. A compound according to any one of embodiments 1 to 22, wherein R⁴ and R⁵ together with the carbon they are attached to form a 5 to 7 membered heterocyclic ring optionally substituted with one, two or three substituents independently selected from alkyl, cyano, halogen, haloalkyl, alkoxy, heteroaryl and alkylheteroaryl.
37. A compound according to any one of embodiments 1 to 22, wherein R⁴ and R⁵ together with the carbon they are attached to form a 5 to 7 membered heterocyclic ring optionally substituted with one, two or three substituents independently selected from alkyl and alkylheteroaryl.
38. A compound according to any one of embodiments 1 to 36, with the proviso that only one of R⁴ and R⁵ can be hydrogen.
39. A compound of formula (I) according to any one of embodiments 1 to 38, selected from
2-(2-Chloro-phenoxy)-6-(5,6-dimethoxy-benzoimidazol-1-yl)-nicotinamide;
6-(5,6-Dimethoxy-benzoimidazol-1-yl)-2-phenylamino-nicotinamide;
2-[[3-amino-1-(3-thienyl)propyl]amino]-6-(5,6-dimethoxybenzimidazol-1-yl)pyridine-3-carboxamide;
2-[(3-amino-1-phenyl-propyl)amino]-6-(5,6-dimethoxybenzimidazol-1-yl)pyridine-3-carboxamide;
   6-(5,6-dimethoxybenzimidazol-1-yl)-2-(2-phenylethylamino)pyridine-3-carboxamide;
   2-(benzylamino)-6-(5,6-dimethoxybenzimidazol-1-yl)pyridine-3-carboxamide;
   6-(5,6-dimethoxybenzimidazol-1-yl)-2-(2-thienylmethylamino)pyridine-3-carboxamide;
   2-[(4-chlorophenyl)methylamino]-6-(5,6-dimethoxybenzimidazol-1-yl)pyridine-3-carboxamide;
   2-[2-(3-chlorophenyl)ethylamino]-6-(5,6-dimethoxybenzimidazol-1-yl)pyridine-3-carboxamide;
   2-[[2-amino-1-(3-chlorophenyl)ethyl]amino]-6-(5,6-dimethoxybenzimidazol-1-yl)pyridine-3-carboxamide;
   2-[[3-amino-1-(3-chlorophenyl)propyl]amino]-6-(5,6-dimethoxybenzimidazol-1-yl)pyridine-3-carboxamide;
   2-[[2-amino-1-(3-thienyl)ethyl]amino]-6-(5,6-dimethoxybenzimidazol-1-yl)pyridine-3-carboxamide;
   methyl 2-(3-chlorophenyl)-6-(5,6-dimethoxy-1H-benzo[d]imidazol-1-yl)nicotinate;
   methyl 6-(5,6-dimethoxy-1H-benzo[d]imidazol-1-yl)-2-(2-oxopiperidin-1-yl)nicotinate;
   methyl 6-(5,6-dimethoxy-1H-benzo[d]imidazol-1-yl)-2-(2-oxopyrrolidin-1-yl)nicotinate;
   6-(5,6-dimethoxy-1H-benzo[d]imidazol-1-yl)-2-(piperidin-1-yl)nicotinic acid;
   2-(3-cyanophenyl)-6-(5,6-dimethoxybenzimidazol-1-yl)-N-ethyl-pyridine-3-carboxamide;
   2-(3-cyanophenyl)-6-(5,6-dimethoxybenzimidazol-1-yl)-N-methyl-pyridine-3-carboxamide;
   2-(3-cyanophenyl)-N-cyclopropyl-6-(5,6-dimethoxybenzimidazol-1-yl)pyridine-3-carboxamide;
   2-(3-cyanophenyl)-6-(5,6-dimethoxybenzimidazol-1-yl)-N-(2,2,2-trifluoroethyl)pyridine-3-carboxamide;
   2-(3-cyanophenyl)-6-(5,6-dimethoxybenzimidazol-1-yl)pyridine-3-carboxamide;
   2-(3-cyano-5-methyl-pyrazol-1-yl)-6-[5-(2-morpholinoethoxy)benzimidazol-1-yl]pyridine-3-carboxamide;
   2-(2,2-difluoro-1-methyl-ethoxy)-6-[5-[(6-methylpyridazin-3-yl)amino]benzimidazol-1-yl]pyridine-3-carboxamide;
   2-(2,2-difluoro-1-methyl-ethoxy)-6-[6-[(6-methylpyridazin-3-yl)amino]benzimidazol-1-yl]pyridine-3-carboxamide;
   2-(3-cyano-5-methyl-pyrazol-1-yl)-6-[5-[[(3S,4R)-4-fluoropyrrolidin-3-yl]amino]benzimidazol-1-yl]pyridine-3-carboxamide;
   methyl 2-(3-methoxy-5-methyl-pyrazol-1-yl)-6-[5-[(6-methylpyridazin-3-yl)amino]benzimidazol-1-yl]pyridine-3-carboxylate;
   1-[3-acetyl-6-[6-[(6-methylpyridazin-3-yl)amino]benzimidazol-1-yl]-2-pyridyl]-5-methyl-pyrazole-3-carbonitrile;
   1-[3-acetyl-6-[5-[(6-methylpyridazin-3-yl)amino]benzimidazol-1-yl]-2-pyridyl]-5-methyl-pyrazole-3-carbonitrile;
   1-[2,4-dimethoxy-6-[5-[(6-methylpyridazin-3-yl)amino]benzimidazol-1-yl]-3-pyridyl]ethanone;
   1-[3-acetyl-6-(6,7-dihydro-5H-pyrrolo[3,2-f]benzimidazol-3-yl)-2-pyridyl]-5-methyl-pyrazole-3-carbonitrile;
   1-[3-acetyl-6-(6,7-dihydro-5H-pyrrolo[2,3-f]benzimidazol-1-yl)-2-pyridyl]-5-methyl-pyrazole-3-carbonitrile;
   (3R,5S)-1-[3-formyl-6-[6-[(6-methylpyridazin-3-yl)amino]benzimidazol-1-yl]-2-pyridyl]-5-methyl-pyrrolidine-3-carbonitrile;
   (3R,5S)-1-[3-formyl-6-[5-[(6-methylpyridazin-3-yl)amino]benzimidazol-1-yl]-2-pyridyl]-5-methyl-pyrrolidine-3-carbonitrile;
   5-methyl-1-[6-[5-[(6-methylpyridazin-3-yl)amino]benzimidazol-1-yl]-3-(2,2,2-trifluoroacetyl)-2-pyridyl]pyrazole-3-carbonitrile;
   1-[3-acetyl-6-[6-keto-7,7-dimethyl-5-(6-methylpyridazin-3-yl)pyrrolo[2,3-f]benzimidazol-1-yl]-2-pyridyl]-5-methyl-pyrazole-3-carbonitrile;
   1-[3-formyl-6-[5-[(6-methylpyridazin-3-yl)amino]benzimidazol-1-yl]-2-pyridyl]-5-methyl-pyrazole-3-carbonitrile;
   1-[3-acetyl-6-[5-[(2-keto-1-methyl-3-pyridyl)amino]benzimidazol-1-yl]-2-pyridyl]-5-methyl-pyrazole-3-carbonitrile;
   1-[3-acetyl-6-[5-(3-methoxy-1-methyl-pyrazol-4-yl)benzimidazol-1-yl]-2-pyridyl]-5-methyl-pyrazole-3-carbonitrile;
   1-[3-acetyl-6-[5-[(6-pyrrolidin-2-ylpyridazin-3-yl)amino]benzimidazol-1-yl]-2-pyridyl]-5-methyl-pyrazole-3-carbonitrile; and
   1-[3-formyl-6-[6-[(6-methylpyridazin-3-yl)amino]benzimidazol-1-yl]-2-pyridyl]-5-methyl-pyrazole-3-carbonitrile;
   or a pharmaceutically acceptable salt thereof.
   40. A compound of formula (I) according to any one of embodiments 1 to 38, selected from
2-(2-Chloro-phenoxy)-6-(5,6-dimethoxy-benzoimidazol-1-yl)-nicotinamide;
6-(5,6-Dimethoxy-benzoimidazol-1-yl)-2-phenylamino-nicotinamide;
2-[[3-amino-1-(3-thienyl)propyl]amino]-6-(5,6-dimethoxybenzimidazol-1-yl)pyridine-3-carboxamide;
2-[(3-amino-1-phenyl-propyl)amino]-6-(5,6-dimethoxybenzimidazol-1-yl)pyridine-3-carboxamide;
6-(5,6-dimethoxybenzimidazol-1-yl)-2-(2-phenylethylamino)pyridine-3-carboxamide;
2-(benzylamino)-6-(5,6-dimethoxybenzimidazol-1-yl)pyridine-3-carboxamide;
6-(5,6-dimethoxybenzimidazol-1-yl)-2-(2-thienylmethylamino)pyridine-3-carboxamide;
2-[(4-chlorophenyl)methylamino]-6-(5,6-dimethoxybenzimidazol-1-yl)pyridine-3-carboxamide;
2-[2-(3-chlorophenyl)ethylamino]-6-(5,6-dimethoxybenzimidazol-1-yl)pyridine-3-carboxamide;
2-[[2-amino-1-(3-chlorophenyl)ethyl]amino]-6-(5,6-dimethoxybenzimidazol-1-yl)pyridine-3-carboxamide;
2-[[3-amino-1-(3-chlorophenyl)propyl]amino]-6-(5,6-dimethoxybenzimidazol-1-yl)pyridine-3-carboxamide;
2-[[2-amino-1-(3-thienyl)ethyl]amino]-6-(5,6-dimethoxybenzimidazol-1-yl)pyridine-3-carboxamide;
methyl 2-(3-chlorophenyl)-6-(5,6-dimethoxy-1H-benzo[d]imidazol-1-yl)nicotinate;
methyl 6-(5,6-dimethoxy-1H-benzo[d]imidazol-1-yl)-2-(2-oxopiperidin-1-yl)nicotinate;
methyl 6-(5,6-dimethoxy-1H-benzo[d]imidazol-1-yl)-2-(2-oxopyrrolidin-1-yl)nicotinate;
6-(5,6-dimethoxy-1H-benzo[d]imidazol-1-yl)-2-(piperidin-1-yl)nicotinic acid;
2-(3-cyanophenyl)-6-(5,6-dimethoxybenzimidazol-1-yl)-N-ethyl-pyridine-3-carboxamide;
2-(3-cyanophenyl)-6-(5,6-dimethoxybenzimidazol-1-yl)-N-methyl-pyridine-3-carboxamide;
2-(3-cyanophenyl)-N-cyclopropyl-6-(5,6-dimethoxybenzimidazol-1-yl)pyridine-3-carboxamide;
2-(3-cyanophenyl)-6-(5,6-dimethoxybenzimidazol-1-yl)-N-(2,2,2-trifluoroethyl)pyridine-3-carboxamide;
2-(3-cyanophenyl)-6-(5,6-dimethoxybenzimidazol-1-yl)pyridine-3-carboxamide;
2-(3-cyano-5-methyl-pyrazol-1-yl)-6-[5-(2-morpholinoethoxy)benzimidazol-1-yl]pyridine-3-carboxamide;
2-(2,2-difluoro-1-methyl-ethoxy)-6-[5-[(6-methylpyridazin-3-yl)amino]benzimidazol-1-yl]pyridine-3-carboxamide;
2-(2,2-difluoro-1-methyl-ethoxy)-6-[6-[(6-methylpyridazin-3-yl)amino]benzimidazol-1-yl]pyridine-3-carboxamide;
2-(3-cyano-5-methyl-pyrazol-1-yl)-6-[5-[[(3S,4R)-4-fluoropyrrolidin-3-yl]amino]benzimidazol-1-yl]pyridine-3-carboxamide;
methyl 2-(3-methoxy-5-methyl-pyrazol-1-yl)-6-[5-[(6-methylpyridazin-3-yl)amino]benzimidazol-1-yl]pyridine-3-carboxylate;
1-[3-acetyl-6-[6-[(6-methylpyridazin-3-yl)amino]benzimidazol-1-yl]-2-pyridyl]-5-methyl-pyrazole-3-carbonitrile; and
1-[3-acetyl-6-[5-[(6-methylpyridazin-3-yl)amino]benzimidazol-1-yl]-2-pyridyl]-5-methyl-pyrazole-3-carbonitrile;
or a pharmaceutically acceptable salt thereof.
   41. A compound of formula (I) according to any one of embodiments 1 to 38, selected from
2-(2-Chloro-phenoxy)-6-(5,6-dimethoxy-benzoimidazol-1-yl)-nicotinamide;
6-(5,6-Dimethoxy-benzoimidazol-1-yl)-2-phenylamino-nicotinamide;
6-(5,6-dimethoxybenzimidazol-1-yl)-2-(2-phenylethylamino)pyridine-3-carboxamide;
2-[[2-amino-1-(3-chlorophenyl)ethyl]amino]-6-(5,6-dimethoxybenzimidazol-1-yl)pyridine-3-carboxamide; and
2-[[2-amino-1-(3-thienyl)ethyl]amino]-6-(5,6-dimethoxybenzimidazol-1-yl)pyridine-3-carboxamide;
   or a pharmaceutically acceptable salt thereof.
   42. A compound of formula (I) according to any one of embodiments 1 to 38, selected from
2-(2,2-difluoro-1-methyl-ethoxy)-6-[5-[(6-methylpyridazin-3-yl)amino]benzimidazol-1-yl]pyridine-3-carboxamide; and
1-[3-acetyl-6-[5-[(6-methylpyridazin-3-yl)amino]benzimidazol-1-yl]-2-pyridyl]-5-methyl-pyrazole-3-carbonitrile;
   or a pharmaceutically acceptable salt thereof.
   43. A compound of formula (I) according to any one of embodiments 1 to 38, selected from
2-(2,2-difluoro-1-methyl-ethoxy)-6-[5-[(6-methylpyridazin-3-yl)amino]benzimidazol-1-yl]pyridine-3-carboxamide;
1-[3-acetyl-6-[5-[(6-methylpyridazin-3-yl)amino]benzimidazol-1-yl]-2-pyridyl]-5-methyl-pyrazole-3-carbonitrile;
(3R,5S)-1-[3-formyl-6-[5-[(6-methylpyridazin-3-yl)amino]benzimidazol-1-yl]-2-pyridyl]-5-methyl-pyrrolidine-3-carbonitrile;
5-methyl-1-[6-[5-[(6-methylpyridazin-3-yl)amino]benzimidazol-1-yl]-3-(2,2,2-trifluoroacetyl)-2-pyridyl]pyrazole-3-carbonitrile; and
1-[3-formyl-6-[5-[(6-methylpyridazin-3-yl)amino]benzimidazol-1-yl]-2-pyridyl]-5-methyl-pyrazole-3-carbonitrile;
   or a pharmaceutically acceptable salt thereof.
   44. A process for the preparation of a compound according to any one of embodiments 1 to 43, comprising one of the following steps:
      (a) the reaction of a compound of formula (B1) or (B2)

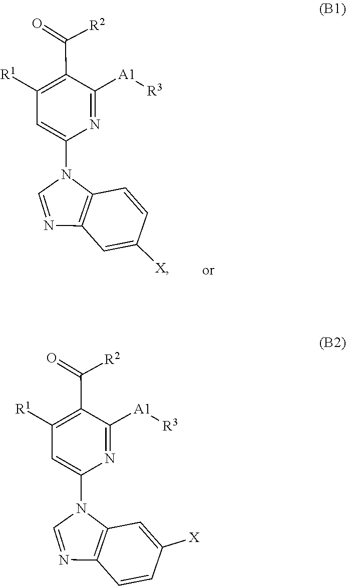

with an amine, in presence of a palladium catalyst and a base;
(b) the reaction of a compound of formula (C1)

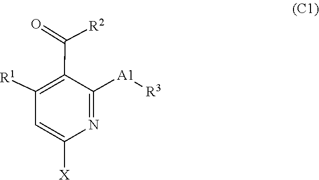

with a compound of formula (C2)

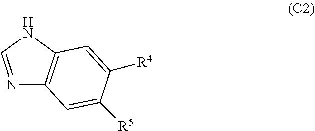

in presence of a base;
(c) the reaction of a compound of formula (D1)

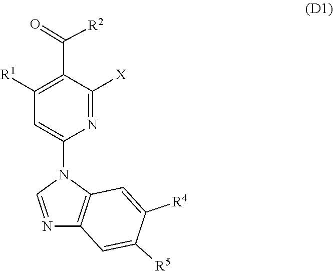

with an amine, in presence of a base; or
(d) the reaction of the compound of formula (D1)
with a compound (D2) in presence of a base and a palladium catalyst; wherein D2 is selected from
(i) optionally substituted aryl boronic acid or ester, and (ii) optionally substituted heteroaryl boronic acid or ester;
   wherein A1, R¹, R², R³, R⁴ and R⁵ are as defined in any one of embodiments 1 to 43, R^{a} is alkyl or cycloalkyl, R^{b} is hydrogen or alkyl, R^{c} is alkyl or cycloalkyl, and X is halogen.
45. A compound according to any one of embodiments 1 to 43 when manufactured according to a process of embodiment 44.
46. A compound of formula (I) according to any one of embodiments 1 to 43 or a pharmaceutically acceptable salt thereof, for use as therapeutically active substance.
47. A pharmaceutical composition comprising a compound of formula (I) according to any one of embodiments 1 to 43 or a pharmaceutically acceptable salt thereof, and a therapeutically inert carrier.
48. The use of a compound of formula (I) according to any one of embodiments 1 to 43 or a pharmaceutically acceptable salt thereof, for the treatment or prophylaxis of rheumatoid arthritis, juvenile rheumatoid arthritis, non-alcoholic steatohepatitis (NASH), primary sclerosing cholangitis, giant cell vasculitis, inflammatory bowel diseases (IBD), atherosclerosis, type 2 diabetes or glomerulonephritis.
49. The use of a compound of formula (I) according to any one of embodiments 1 to 43 or a pharmaceutically acceptable salt thereof, for the preparation of a medicament for the treatment or prophylaxis rheumatoid arthritis, juvenile rheumatoid arthritis, non-alcoholic steatohepatitis (NASH), primary sclerosing cholangitis, giant cell vasculitis, inflammatory bowel diseases (IBD), atherosclerosis, type 2 diabetes or glomerulonephritis.
50. A compound of formula (I) according to any one of embodiments 1 to 43 or a pharmaceutically acceptable salt thereof, for use in the treatment or prophylaxis of rheumatoid arthritis, juvenile rheumatoid arthritis, non-alcoholic steatohepatitis (NASH), primary sclerosing cholangitis, giant cell vasculitis, inflammatory bowel diseases (IBD), atherosclerosis, type 2 diabetes or glomerulonephritis.
51. A method for the treatment or prophylaxis of rheumatoid arthritis, juvenile rheumatoid arthritis, non-alcoholic steatohepatitis (NASH), primary sclerosing cholangitis, giant cell vasculitis, inflammatory bowel diseases (IBD), atherosclerosis, type 2 diabetes or glomerulonephritis, which method comprises administering an effective amount of a compound of formula (I) according to any one of embodiments 1 to 43 or a pharmaceutically acceptable salt thereof, to a patient in need thereof.

1. A compound of formula (I) 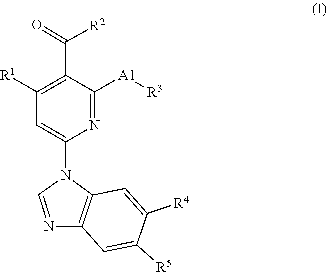
wherein
R¹ is hydrogen or alkoxy;
R² is hydrogen, alkyl, amino, alkylamino, dialkylamino, haloalkyl, haloalkylamino cycloalkylamino, hydroxy, alkoxy, cycloalkyl, cycloalkyloxy or haloalkoxy;
A1 is —O—, —NR⁶— or a bond;
R⁶ is hydrogen or alkyl;
R³ is alkyl, haloalkyl, hydroxyalkyl, heterocycloalkyl, heteroaryl, phenyl, heteroarylalkyl, phenylalkyl, cycloalkyl, cycloalkylalkyl, (amino)(phenyl)alkyl, (amino)(halophenyl)alkyl or (amino)(heteroaryl)alkyl, wherein heterocycloalkyl, heteroaryl, phenyl, heteroarylalkyl, phenylalkyl, cycloalkyl and cycloalkylalkyl are optionally substituted with 1, 2 or 3 substituents independently selected from R⁷;
each R⁷ is independently selected from alkoxy, alkylamino, alkyl, aminocarbonyl, amino, cyano, cycloalkylamino, haloalkyl, halocycloalkyl, halogen, heteroaryl, hydroxycarbonylamino, alkoxyalkyl, alkylaminocarbonyl, alkylsulfonyl, aminocarbonyl, hydroxy, cycloalkylalkyl, haloalkoxy, heterocycloalkyl and cycloalkyl;
R⁴ is hydrogen, alkyl, halogen, cyano, haloalkyl, alkoxy, alkoxyalkyl, dialkylaminoalkyl, dialkylamino, alkylamino, alkylaminoalkyl, cycloalkyl, cycloalkylamino, cycloalkyloxy, cycloalkylalkyl, heteroarylalkyl, heteroarylamino, heteroaryloxy, heterocycloalkyl, heterocycloalkylamino, heterocycloalkyloxy or heterocycloalkylalkyl; wherein cycloalkyl, cycloalkylamino, cycloalkyloxy, cycloalkylalkyl, heteroarylalkyl, heteroarylamino, heteroaryloxy, heterocycloalkyl, heterocycloalkylamino, heterocycloalkyloxy and heterocycloalkylalkyl are optionally substituted with 1, 2 or 3 substituents independently selected from R⁸;
each R⁸ is independently selected from alkyl, halogen, cyano, alkylsulfonyl, alkylaminocarbonyl, heterocycloalkyl and alkoxyheterocycloalkylalkyl;
R⁵ is hydrogen, alkyl, halogen, cyano, haloalkyl, alkoxy, alkoxyalkyl, dialkylaminoalkyl, dialkylamino, alkylamino, alkylaminoalkyl, alkylsulfonyl, cycloalkyl, cycloalkylamino, cycloalkyloxy, cycloalkylalkyl, heteroarylalkyl, heteroarylamino, heteroaryloxy, heterocycloalkyl, heterocycloalkylamino, heterocycloalkyloxy or heterocycloalkylalkyl; wherein cycloalkyl, cycloalkylamino, cycloalkyloxy, cycloalkylalkyl, heteroarylalkyl, heteroarylamino, heteroaryloxy, heterocycloalkyl, heterocycloalkylamino, heterocycloalkyloxy and heterocycloalkylalkyl are optionally substituted with 1, 2 or 3 substituents independently selected from R⁹;
or R⁴ and R⁵ together with the carbon they are attached to form a 5 to 7 membered heterocyclic ring optionally substituted with one, two or three substituents independently selected from alkyl, cyano, halogen, haloalkyl, alkoxy, heteroaryl and alkylheteroaryl;
each R⁹ is independently selected from alkoxy, halogen, dialkylaminocarbonyl, alkyl, alkoxyalkoxy, alkoxyheterocycloalkylalkyl, alkoxyheterocycloalkylcarbonyl, haloalkyl, haloalkoxy, heterocycloalkylalkoxy, heterocycloalkyl, heterocycloalkyloxy, hydroxy, alkylheterocycloalkyl, alkylheterocycloalkylalkyl, heterocycloalkylalkyl, alkylsulfonyl, (alkyl)heterocycloalkyl, alkylheterocycloalkyloxy, heterocycloalkylheterocycloalkyl, (heterocycloalkyl)heterocycloalkyl, CH₃—O—(CH₂—CH₂—O)₇—, alkylaminocarbonyl and cyano;
or a pharmaceutically acceptable salt thereof.

2. A compound according to claim 1, wherein R¹ is hydrogen or methoxy.

3. A compound according to claim 1, wherein R² is hydrogen, methyl, amino, methylamino, ethylamino, dimethylamino, trifluoromethyl, trifluoromethylamino, cyclopropylamino, hydroxy or methoxy.

4. A compound according to claim 1, wherein R² is amino or alkyl.

5. A compound according to claim 1, wherein A1 is —O— or a bond.

6. A compound according to claim 1, wherein R³ is alkyl, haloalkyl, heterocycloalkyl, heteroaryl, phenyl, phenylalkyl, (amino)(phenyl)alkyl, (amino)(halophenyl)alkyl or (amino)(heteroaryl)alkyl, wherein heterocycloalkyl, heteroaryl, phenyl, heteroarylalkyl and phenylalkyl are optionally substituted with 1, 2 or 3 substituents independently selected from R⁷; and
wherein each R⁷ is independently selected from halogen, alkyl, cyano, alkoxy and haloalkyl.

7. A compound according to claim 1, wherein R³ is methyl difluoropropyl, phenyl, phenylmethyl, phenylethyl, 2-amino-1-(3-chlorophenyl)ethyl, 3-amino-1-phenyl-propyl, 3-amino-1-(3-thienyl)propyl, 2-thienylmethyl, heterocycloalkyl or heteroaryl, wherein heterocycloalkyl, heteroaryl, phenyl, phenylmethyl and phenylethyl are optionally substituted with 1, 2 or 3 substituents independently selected from R⁷; and
wherein each R⁷ is independently selected from chloro, methyl, cyano, methoxy and difluoromethyl.

8. A compound according to claim 1, wherein
R⁴ is hydrogen, halogen, cyano, haloalkyl, alkoxy, alkoxyalkyl, dialkylaminoalkyl, dialkylamino, alkylamino, alkylaminoalkyl, cycloalkyl, cycloalkylamino, cycloalkyloxy, cycloalkylalkyl, heteroarylalkyl, heteroarylamino, heteroaryloxy, heterocycloalkyl, heterocycloalkylamino, heterocycloalkyloxy or heterocycloalkylalkyl; wherein cycloalkyl, cycloalkylamino, cycloalkyloxy, cycloalkylalkyl, heteroarylalkyl, heteroarylamino, heteroaryloxy, heterocycloalkyl, heterocycloalkylamino, heterocycloalkyloxy and heterocycloalkylalkyl are optionally substituted with 1, 2 or 3 substituents independently selected from R⁸;
each R⁸ is independently selected from alkyl, halogen, cyano, alkylsulfonyl, alkylaminocarbonyl, heterocycloalkyl and alkoxyheterocycloalkylalkyl;
R⁵ is hydrogen, halogen, cyano, haloalkyl, alkoxy, alkoxyalkyl, dialkylaminoalkyl, dialkylamino, alkylamino, alkylaminoalkyl, alkylsulfonyl, cycloalkyl, cycloalkylamino, cycloalkyloxy, cycloalkylalkyl, heteroarylalkyl, heteroarylamino, heteroaryloxy, heterocycloalkyl, heterocycloalkylamino, heterocycloalkyloxy or heterocycloalkylalkyl; wherein cycloalkyl, cycloalkylamino, cycloalkyloxy, cycloalkylalkyl, heteroarylalkyl, heteroarylamino, heteroaryloxy, heterocycloalkyl, heterocycloalkylamino, heterocycloalkyloxy and heterocycloalkylalkyl are optionally substituted with 1, 2 or 3 substituents independently selected from R⁹;
or R⁴ and R⁵ together with the carbon they are attached to form a 5 to 7 membered heterocyclic ring optionally substituted with one, two or three substituents independently selected from alkyl, cyano, halogen, haloalkyl, alkoxy, heteroaryl and alkylheteroaryl;
each R⁹ is independently selected from alkoxy, halogen, dialkylaminocarbonyl, alkyl, alkoxyalkoxy, alkoxyheterocycloalkylalkyl, alkoxyheterocycloalkylcarbonyl, haloalkyl, haloalkoxy, heterocycloalkylalkoxy, heterocycloalkyl, heterocycloalkyloxy, hydroxy, alkylheterocycloalkyl, alkylheterocycloalkylalkyl, heterocycloalkylalkyl, alkylsulfonyl, (alkyl)heterocycloalkyl, alkylheterocycloalkyloxy, heterocycloalkylheterocycloalkyl, (heterocycloalkyl)heterocycloalkyl, CH₃—O—(CH₂—CH₂—O)₇—, alkylaminocarbonyl and cyano;
with the proviso that only one of R⁴ and R⁵ can be hydrogen.

9. A compound according to claim 1, wherein
R⁴ is hydrogen, fluoro, cyano, trifluoromethyl, methoxy, methoxyethyl, dimethylaminoethyl, cyclopropylcarbonyl, morpholinoethyl, (1,1-dioxo-1,2-thiazolidin-2-yl)methyl, (2-oxopyrrolidin-1-yl)methyl, (2-oxo-1-piperidyl)methyl, heteroaryl or heterocycloalkyl, wherein heteroaryl and heterocycloalkyl are optionally substituted with 1, 2 or 3 substituents independently selected from R⁸;
each R⁸ is independently selected from methyl, fluoro, cyano, methylsulfonyl, oxetan-3-yl and (3-methoxyazetidin-1-yl)methyl;
R⁵ is hydrogen, alkoxy, heterocycloalkylalkoxy, heterocycloalkylamino, heteroarylamino, heteroarylalkyl or heteroaryl, wherein heterocycloalkylalkoxy, heterocycloalkylamino, heteroarylamino, heteroarylalkyl and heteroaryl are optionally substituted with 1, 2 or 3 substituents independently selected from R⁹;
or R⁴ and R⁵ together with the carbon they are attached to form a 5 to 7 membered heterocyclic ring optionally substituted with one, two or three substituents independently selected from alkyl and alkylheteroaryl;
each R⁹ is independently selected from alkyl, alkoxy, halogen, haloalkyl, dialkylaminocarbonyl, heterocycloalkyl and (heterocycloalkyl)heterocycloalkyl;
with the proviso that only one of R⁴ and R⁵ can be hydrogen.

10. A compound according to claim 1, wherein R⁴ is hydrogen.

11. A compound according to claim 1, wherein R⁵ is pyridazin-3-yl optionally substituted with alkyl.

12. A compound of formula (I) according to claim 1, selected from
2-(2-Chloro-phenoxy)-6-(5,6-dimethoxy-benzoimidazol-1-yl)-nicotinamide;
6-(5,6-Dimethoxy-benzoimidazol-1-yl)-2-phenylamino-nicotinamide;
2-[[3-amino-1-(3-thienyl)propyl]amino]-6-(5,6-dimethoxybenzimidazol-1-yl)pyridine-3-carboxamide;
2-[(3-amino-1-phenyl-propyl)amino]-6-(5,6-dimethoxybenzimidazol-1-yl)pyridine-3-carboxamide;
6-(5,6-dimethoxybenzimidazol-1-yl)-2-(2-phenylethylamino)pyridine-3-carboxamide;
2-(benzylamino)-6-(5,6-dimethoxybenzimidazol-1-yl)pyridine-3-carboxamide;
6-(5,6-dimethoxybenzimidazol-1-yl)-2-(2-thienylmethylamino)pyridine-3-carboxamide;
2-[(4-chlorophenyl)methylamino]-6-(5,6-dimethoxybenzimidazol-1-yl)pyridine-3-carboxamide;
2-[2-(3-chlorophenyl)ethylamino]-6-(5,6-dimethoxybenzimidazol-1-yl)pyridine-3-carboxamide;
2-[[2-amino-1-(3-chlorophenyl)ethyl]amino]-6-(5,6-dimethoxybenzimidazol-1-yl)pyridine-3-carboxamide;
2-[[3-amino-1-(3-chlorophenyl)propyl]amino]-6-(5,6-dimethoxybenzimidazol-1-yl)pyridine-3-carboxamide;
2-[[2-amino-1-(3-thienyl)ethyl]amino]-6-(5,6-dimethoxybenzimidazol-1-yl)pyridine-3-carboxamide;
methyl 2-(3-chlorophenyl)-6-(5,6-dimethoxy-1H-benzo[d]imidazol-1-yl)nicotinate;
methyl 6-(5,6-dimethoxy-1H-benzo[d]imidazol-1-yl)-2-(2-oxopiperidin-1-yl)nicotinate;
methyl 6-(5,6-dimethoxy-1H-benzo[d]imidazol-1-yl)-2-(2-oxopyrrolidin-1-yl)nicotinate;
6-(5,6-dimethoxy-1H-benzo[d]imidazol-1-yl)-2-(piperidin-1-yl)nicotinic acid;
2-(3-cyanophenyl)-6-(5,6-dimethoxybenzimidazol-1-yl)-N-ethyl-pyridine-3-carboxamide;
2-(3-cyanophenyl)-6-(5,6-dimethoxybenzimidazol-1-yl)-N-methyl-pyridine-3-carboxamide;
2-(3-cyanophenyl)-N-cyclopropyl-6-(5,6-dimethoxybenzimidazol-1-yl)pyridine-3-carboxamide;
2-(3-cyanophenyl)-6-(5,6-dimethoxybenzimidazol-1-yl)-N-(2,2,2-trifluoroethyl)pyridine-3-carboxamide;
2-(3-cyanophenyl)-6-(5,6-dimethoxybenzimidazol-1-yl)pyridine-3-carboxamide;
2-(3-cyano-5-methyl-pyrazol-1-yl)-6-[5-(2-morpholinoethoxy)benzimidazol-1-yl]pyridine-3-carboxamide;
2-(2,2-difluoro-1-methyl-ethoxy)-6-[5-[(6-methylpyridazin-3-yl)amino]benzimidazol-1-yl]pyridine-3-carboxamide;
2-(2,2-difluoro-1-methyl-ethoxy)-6-[6-[(6-methylpyridazin-3-yl)amino]benzimidazol-1-yl]pyridine-3-carboxamide;
2-(3-cyano-5-methyl-pyrazol-1-yl)-6-[5-[[(3S,4R)-4-fluoropyrrolidin-3-yl]amino]benzimidazol-1-yl]pyridine-3-carboxamide;
methyl 2-(3-methoxy-5-methyl-pyrazol-1-yl)-6-[5-[(6-methylpyridazin-3-yl)amino]benzimidazol-1-yl]pyridine-3-carboxylate;
1-[3-acetyl-6-[6-[(6-methylpyridazin-3-yl)amino]benzimidazol-1-yl]-2-pyridyl]-5-methyl-pyrazole-3-carbonitrile;
1-[3-acetyl-6-[5-[(6-methylpyridazin-3-yl)amino]benzimidazol-1-yl]-2-pyridyl]-5-methyl-pyrazole-3-carbonitrile;
1-[2,4-dimethoxy-6-[5-[(6-methylpyridazin-3-yl)amino]benzimidazol-1-yl]-3-pyridyl]ethanone;
1-[3-acetyl-6-(6,7-dihydro-5H-pyrrolo[3,2-f]benzimidazol-3-yl)-2-pyridyl]-5-methyl-pyrazole-3-carbonitrile;
1-[3-acetyl-6-(6,7-dihydro-5H-pyrrolo[2,3-f]benzimidazol-1-yl)-2-pyridyl]-5-methyl-pyrazole-3-carbonitrile;
(3R,5S)-1-[3-formyl-6-[6-[(6-methylpyridazin-3-yl)amino]benzimidazol-1-yl]-2-pyridyl]-5-methyl-pyrrolidine-3-carbonitrile;
(3R,5S)-1-[3-formyl-6-[5-[(6-methylpyridazin-3-yl)amino]benzimidazol-1-yl]-2-pyridyl]-5-methyl-pyrrolidine-3-carbonitrile;
5-methyl-1-[6-[5-[(6-methylpyridazin-3-yl)amino]benzimidazol-1-yl]-3-(2,2,2-trifluoroacetyl)-2-pyridyl]pyrazole-3-carbonitrile;
1-[3-acetyl-6-[6-keto-7,7-dimethyl-5-(6-methylpyridazin-3-yl)pyrrolo[2,3-f]benzimidazol-1-yl]-2-pyridyl]-5-methyl-pyrazole-3-carbonitrile;
1-[3-formyl-6-[5-[(6-methylpyridazin-3-yl)amino]benzimidazol-1-yl]-2-pyridyl]-5-methyl-pyrazole-3-carbonitrile;
1-[3-acetyl-6-[5-[(2-keto-1-methyl-3-pyridyl)amino]benzimidazol-1-yl]-2-pyridyl]-5-methyl-pyrazole-3-carbonitrile;
1-[3-acetyl-6-[5-(3-methoxy-1-methyl-pyrazol-4-yl)benzimidazol-1-yl]-2-pyridyl]-5-methyl-pyrazole-3-carbonitrile;
1-[3-acetyl-6-[5-[(6-pyrrolidin-2-ylpyridazin-3-yl)amino]benzimidazol-1-yl]-2-pyridyl]-5-methyl-pyrazole-3-carbonitrile; and
1-[3-formyl-6-[6-[(6-methylpyridazin-3-yl)amino]benzimidazol-1-yl]-2-pyridyl]-5-methyl-pyrazole-3-carbonitrile;
or a pharmaceutically acceptable salt thereof.

13. A process for the preparation of a compound according to claim 1, comprising one of the following steps:
(a) the reaction of a compound of formula (B1) or (B2) 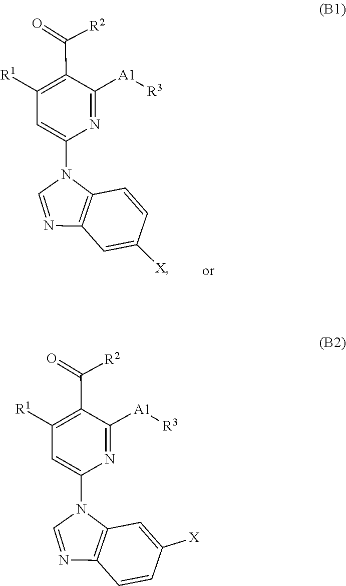
with an amine, in presence of a palladium catalyst and a base;
(b) the reaction of a compound of formula (C1) 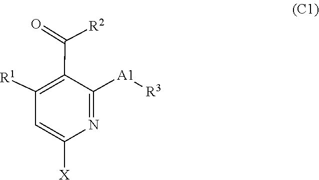
with a compound of formula (C2) 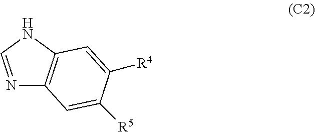
in presence of a base;
(c) the reaction of a compound of formula (D1) 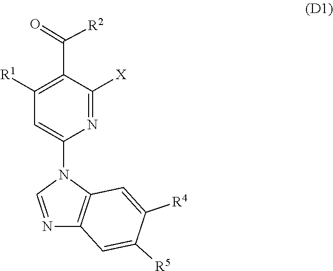
with an amine, in presence of a base; or
(d) the reaction of the compound of formula (D1)
with a compound (D2) in presence of a base and a palladium catalyst; wherein D2 is selected from (i) optionally substituted aryl boronic acid or ester, and (ii) optionally substituted heteroaryl boronic acid or ester;
wherein A1, R¹, R², R³, R⁴ and R⁵ are as defined in any one of claims 1 to 12, R^{a} is alkyl or cycloalkyl, R^{b} is hydrogen or alkyl, R^{c} is alkyl or cycloalkyl, and X is halogen.

14. A pharmaceutical composition comprising a compound of formula (I) according to claim 1 or a pharmaceutically acceptable salt thereof, and a therapeutically inert carrier.

15. A method for the treatment or prophylaxis of rheumatoid arthritis, juvenile rheumatoid arthritis, non-alcoholic steatohepatitis (NASH), primary sclerosing cholangitis, giant cell vasculitis, inflammatory bowel diseases (IBD), atherosclerosis, type 2 diabetes or glomerulonephritis, which method comprises administering an effective amount of a compound of formula (I) according to claim 1 or a pharmaceutically acceptable salt thereof, to a patient in need thereof.
